(19) United States
(12) Patent Application Publication
(10) Pub. No.: US 2022/0117917 A1
(43) Pub. Date: Apr. 21, 2022

(54) PH-ACTIVATED NANOPARTICLES

(71) Applicants: Ohio State Innovation Foundation, Columbus, OH (US); University of Maryland, College Park, MD (US); Indiana University Research and Technology Corporation, Indianapolis, IN (US)

(72) Inventors: Xiongbin Lu, Carmal, IN (US); Xiaoming He, Bethesda, MD (US); Jiangsheng Xu, Greenbelt, MD (US)

(21) Appl. No.: 17/289,425

(22) PCT Filed: Oct. 30, 2019
(86) PCT No.: PCT/US2019/058907

§ 371 Date: Apr. 28, 2021

Related U.S. Application Data
(60) Provisional application No. 62/752,851, filed on Oct. 30, 2018.

(51) Int. Cl. A61K 31/155; A61K 9/127; A61K 47/36; A61K 47/24; A61K 47/18; A61K 47/02; A61K 31/7105; A61K 31/4436; A61K 31/337; A61K 31/4745; A61K 31/704; A61K 47/34; A61K 47/10; A61P 35/00

(52) U.S. Cl. CPC A61K 31/155; A61K 9/127; A61K 47/36; A61K 47/24; A61K 47/183; A61K 47/02; A61P 35/00; A61K 31/4436; A61K 31/337; A61K 31/4745; A61K 31/704; A61K 47/34; A61K 47/10; A61K 31/7105

(57) ABSTRACT

Disclosed herein is a pH activated nanoparticle that can be used to deliver labile therapeutic or diagnostic agents to the cytoplasm of cells. These nanoparticles allow the agents to escape the endosome by releasing a gas in an amount effective to disrupt the endosome and release the agents into the cytoplasm. The disclosed nanoparticles have a shell, such as a phospholipid bilayer shell, and a core containing a gas bound to a substrate by a pH sensitive interaction. Also disclosed herein is are methods for delivering a pH sensitive cargo to the cytoplasm of a cell, treating triple negative breast cancer (TNBC) in a subject, and treating HER2+ breast cancer in a subject.

### CROSS-REFERENCE TO RELATED APPLICATIONS

This application claims benefit of U.S. Provisional Application No. 62/752,851, filed Oct. 30, 2018, which is hereby incorporated herein by reference in its entirety.

### STATEMENT REGARDING FEDERALLY SPONSORED RESEARCH OR DEVELOPMENT

This invention was made with Government Support under Grant Nos. CA206366 and CA203737 awarded by the National Institutes of Health. The Government has certain rights in the invention.

### SEQUENCE LISTING

This application contains a sequence listing filed in electronic form as an ASCII.txt file entitled “321501_2370_Sequence_Listing_ST25” created on Oct. 25, 2019. The content of the sequence listing is incorporated herein in its entirety.

### BACKGROUND

Breast cancer is the leading cause of death in women. Genetic background and environmental factors are believed to contribute to the complexity of human breast cancer. Over the past decades, a large body of literature has demonstrated that gene expression profile is a useful tool to define the signature of breast cancer and predict the prognosis or response to treatment. Genomic studies have identified distinct breast cancer subtypes with differences in survival and response to therapy, including luminal A, luminal B, basal-like, HER2+ and Claudin-low subtypes (Lim E, et al. Nat Med 2009 15:907-913; Prat A, et al. Nat Med 2009 15:842-844). The human epidermal growth factor receptor 2 (HER2) belongs to the ErbB family of receptor tyrosine kinases, which is overexpressed in 20%-30% of human breast cancers (Yu D, et al. Oncogene 2000 19:6115-6121; Slamon D J, et al. Science 1987 235:177-182). HER2 overexpression leads to aggressive cancer phenotype and poor patient survival. Trastuzumab is a humanized antibody that is rationally designed for HER2-targeted therapy. It shows considerable clinical efficacy and extends the overall survival of certain patients with HER2-positive breast cancer. However, the overall response rate to trastuzumab-containing therapies remains modest: only 26% when used as single therapy and 40-60% when used in combination with chemotherapy (Seidman A D, et al. J Clin Oncol 2008 26:1642-1649; Slamon D J, et al. N Engl J Med 2001 344:783-792; Vogel C L, et al. J Clin Oncol 2002 20:719-726).

Triple negative breast cancer (TNBC) is negative for the expression of oestrogen and progesterone receptors, and absent of human epidermal growth factor receptor 2 (HER2) overexpression (Dent, R, et al. Clin. Cancer Res. 2007 13(15):4429-4434; Foulkes, W D, et al. N. Engl. J. Med. 2010 363(20):1938-1948; Shah, S P, et al. Nature 2012 486(7403):395-399). These receptors are molecular targets for treating breast cancer. As a result, other than olaparib, a poly(ADP-ribose) polymerase inhibitor that can benefit a small subset of TNBC patients with BRCA mutation, no approved targeted therapies are available for most TNBC patients. Standard chemotherapy is the only approved option, but it is ineffective with undesired side effects (Denkert, C, et al. Lancet 2017 389(10087):2430-2442; Mayer, E L, et al. J. Clin. Oncol. 2016 34(28):3369-3371). Therefore, new targeted therapies are critically needed for TNBC.

RNA interference (RNAi) with small interfering RNA (siRNA) can be used to target virtually any genes (Novina, C D., et al. Nature 2004 430(6996):161; Liang, C., et al. Nat. Med. 2015 21:288; Cox, A D., et al. Nat. Rev. Drug Discov. 2014 13:828). It shows therapeutic potential for treating various diseases including cancer (Paul, C P, et al. Nat. Biotechnol. 2002 20:505; Morris, K V, et al. Science 2004 305(5688):1289-1292; Kumar, P, et al. Nature 2007 448:39). However, few RNAi therapeutics have entered phase II/III clinical trials (Wittrup, A, et al. Nat. Rev. Genet. 2015 16:543; Bobbin, M L, et al. Annu. Rev. Pharmacol. Toxicol. 2016 56(1):103-122; Dahlman, J E, et al. Nat. Nanotechnol. 2014 9:648; Zuckerman, J E, et al. Nat. Rev. Drug Discov. 2015 14(12):843-856). This is because naked RNAs including siRNA, have poor stability in blood, don't enter cells, and are instable in the endo/lysosomes (Wang, H, et al. Adv. Mater. 2016 28(2):347-355). Nanotechnology has demonstrated potential for overcoming the challenges facing RNAi based therapy (Cui, J, et al. Nat. Commun. 2017 8(1):191; Lee, H, et al. Nat. Nanotechnol. 2012 7(6):389; Kong, H J, et al. Nat. Rev. Drug. Discov. 2007 6(6):455-463; Shu, D, et al. ACS Nano 2015 9(10):9731-9740; Adams, B D, et al. Cancer Res. 2016 76(4), 927-939). Nanoparticles can prolong the half-life of RNAs in blood, preferentially accumulate in tumour, enhance cellular uptake, and allow for stimuli-responsive release of payload (Guo, X, et al. Acc. Chem. Res. 2012 45(7):971-979; Zhou, J, et al. Nat. Mater. 2011 11:82; Wang, H, et al. Nanomedicine 2016 11(2):103-106; Farokhzad, O C, et al. ACS Nano 2009 3(1):16-20; Lu, Y, et al. Nat. Rev. Mater. 2016 2:16075). However, the RNAs released from nanoparticles could be easily degraded in endosomes/lysosomes after cellular uptake by endocytosis (Wang, H, et al. Adv. Mater. 2016 28(2):347-355; Kim, H J, et al. Adv. Drug. Deliv. Rev. 2016 104:61-77). Therefore, it is crucial to achieve endo/lysosomal escape for effective release of siRNAs into the cytosol so that they can interact with the RNAi machinery to induce gene silencing (El Andaloussi, S, et al. Nat. Rev. Drug Discov. 2013 12:347; Maas, S L N, et al. Trends Cell Biol. 2017 27(3):172-188; Alvarez-Erviti, L, et al. Nat. Biotechnol. 2011 29:341; Zhao, Y, et al. Nat. Commun. 2016 7:11822).

### SUMMARY

Disclosed herein is a pH activated nanoparticle that can be used to deliver labile therapeutic or diagnostic agents to the cytoplasm of cells. These nanoparticles allow the agents to escape the endosome by releasing a gas in an amount effective to disrupt the endosome and release the agents into the cytoplasm. The disclosed nanoparticles have a shell, such as a phospholipid bilayer shell, and a core containing a gas bound to a substrate by a pH sensitive interaction.

The substrate is in some embodiments chitosan-guanidine (CG) or chitosan-arginine (CA). Other suitable substrates include metformin and calcium carbonate. The substrate must be biocompatible and able to bind a gas, such as carbon dioxide. This bond must be pH sensitive so that the gas is released at a pH of 6.5 to 4.5 found in the endoosom.

The core of the nanoparticle can further contain a labile therapeutic or diagnostic agent that would otherwise degrade at endosomal pH. For example, the pH sensitive therapeutic or diagnostic agent can be an RNA or DNA oligonucleotide, such as an mRNA, ncRNA, siRNA, miRNA, and shRNA oligonucleotide. The agent can also be a peptide or labile small molecule.

In some embodiments, the therapeutic agent is a POLR2A-targeting siRNA (siPol2). In some embodiments, the therapeutic agent is an anti-miR-21 oligonucleotide. In these embodiments, the core can further contain a small molecule inhibitor against WIP1, such as GSK2830371. In some embodiments, the core contains the small molecule inhibitors paclitaxel, camptothecin, doxorubicin, or any combination thereof.

Natural and synthetic phospholipids that can form phospholipid bilayers for nanoparticle shells are known in the art. In some embodiments, the phospholipid is dipalmitoyl phosphatidylcholine (DPPC) or dioleoyl phosphatidylcholine (DOPC). The nanoparticle shell can contain other materials, such as polymers and surfactants. In some embodiments, the shell contains poly(lactic-co-glycolic acid) (PLGA), such as PEGylated PLGA. In some embodiments, the shell contains a poloxamer, such as poloxamer 407.

Also disclosed herein is a method for treating triple negative breast cancer (TNBC) in a subject that involves administering to the subject a therapeutically effective amount of the pH activated nanoparticle disclosed herein where the therapeutic agent is a POLR2A-targeting siRNA (siPol2). In some embodiments, the TNBC has a TP53 gene mutation or deletion.

Also disclosed herein is a method for delivering a pH sensitive cargo to the cytoplasm of a cell that involves loading the pH sensitive cargo into a pH activated nanoparticle disclosed herein, and contacting the cell with the loaded nanoparticle.

Also disclosed herein is a method for treating HER2+ breast cancer in a subject that involves administering to the subject a therapeutically effective amount of an anti-miR-21 oligonucleotide and a small molecule inhibitor against WIP1. In some embodiments, the method further involves administering to the subject a therapeutically effective amount of an anti-HER2 antibody, such as trastuzumab. In some embodiments, the HER2+ breast cancer is trastuzumab-resistant. In some embodiments, the anti-miR-21 oligonucleotide and a small molecule inhibitor against WIP1 are loaded in a pH activated nanoparticle disclosed herein.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIGS. 1A to 1K show POLR2A is almost always deleted together with TP53 in triple negative breast cancers. FIGS. 1A to 1B show genomic alterations of TP53 (hemizygous deletion and heterozygous deletion, and point mutation) in The Cancer Genome Atlas primary (FIG. 1A) and metastatic (FIG. 1B) breast cancer dataset determined by cBioportal (n=2,051 and 213 biologically independent samples for primary and metastatic cancer, respectively). FIG. 1C show the frequency of hemizygous TP53 loss in five major human breast cancer subtypes (n indicates the number of biologically independent samples for each subtype). HER2: Human epidermal growth factor receptor 2. FIG. 1D is a heatmap of genomic segment copy-number abnormalities (log-ratio measurements) of human chromosome 17 in triple negative breast cancers (TNBCs) as well as all the other invasive non-TNBC breast carcinomas. Positive log-ratios indicate degree of copy number gain (red) whereas negative values present the loss (blue). FIG. 1E is a schematic diagram showing genes adjacent to TP53 in human genome. FIG. 1F shows concomitant deletion of POLR2A in TNBC containing hemizygous loss of TP53. FIG. 1G shows correlation between gene expression and copy number variation for POLR2A and TP53 genes in breast tumours (n indicates the number of biologically independent samples). The Box-Whisker plots present a five-number summary: minima, lower quartile, centre, upper quartile, and maxima. Error bars denote mean±s.d., and statistical significance was assessed by Student's t-test (unpaired and two-tailed). FIG. 1H shows the frequency of TNBC patients with hemizygous TP53 deletion at stages I, II, and III, respectively. FIG. 1I shows protein levels of POLR2A and p53 in seven different human TNBC cell lines (the experiments were repeated three times independently). FIGS. 1J to 1K show quantification of POLR2A expression in human breast normal, POLR2A^{neutral} and POLR2A^{loss} TNBC tumour tissue samples (FIG. 1J), and the representative images are shown in FIG. 1K. In FIG. 1K, two representative samples are shown for each of the three of tissues and the experiments were repeated three times independently. Error bars denote mean±s.d., and statistical significance was assessed by Student's t-test (unpaired, two-tailed). **: p<0.01 and ***: p<0.001. Scale bar, 100 μm. METABRIC and France indicate the origin of the data in The Cancer Genome Atlas.

FIGS. 2A to 2F show synthesis and characterization of nanoparticles for stabilizing POLR2A targeting siRNA. FIG. 2A is a Schematic illustration of the core-shell structured nanoparticle that synthesized using the double-emulsion approach of water-in-oil-in-water (w/o/w). The aqueous phase containing POLR2A targeting siRNA (siPol2) and chitosan-guanidinate-CO₂ (CG-CO₂) was encapsulated in the core of the nanoparticle. Poly(lactic-co-glycolic acid) (PLGA) and dipalmitoylphosphatidylcholine (DPPC) in the organic phase (oil, dichloromethane) were used to form the shell of the nanoparticle. FIG. 2B contain illustrations of the chemical reactions for modification of chitosan with guanidine and for the pH dependent capture/release of CO₂ with the guanidine-modified chitosan. FIG. 2C shows transmission electron microscopy (TEM) images of the nanoparticles under different pH conditions for 3 h at 37° C. The nanoparticles maintain a spherical morphology and core-shell structure at pH 7.4. At pH 6.0, the nanoparticles become enlarged with a damaged shell (arrows indicated defects on the shell), probably due to the generation of CO₂ inside the nanoparticles. The change is more evident at pH 5.0 with the appearance of an additional peak for the enlarged nanoparticles. Scale bars: 500 nm and 100 nm for low and high magnification images shown in the top and bottom rows, respectively. FIG. 2D shows nanoparticle size distribution determined by dynamic light scattering (DLS) at pH 7.4, 6.0, and 5.0. FIG. 2E is an electrophoretic gel assay showing the low pH-responsive release of siPol2 from siPol2@NPs in PBS (RNase free). FIG. 2F is an electrophoretic gel assay showing the stability of free siPol2 compared to siPol2 encapsulated in the nanoparticles (siPol2@NPs) after incubating them in serum at 37° C. for up to 24 h. The observable signal of siPol2@NPs at 24 hours indicates that the nanoparticle encapsulation could protect siPol2 from degradation in serum, while the signal of free siPol2 in serum disappeared quickly. The experiments for FIGS. 2C to 2F were repeated three times independently.

FIGS. 3A and 3B show low pH activated endo/lysosomal escape. FIG. 3A shows typical confocal images of MDA-MB-453 TNBC cells incubated with Cy5.5-siPol2@NPs (with CO₂) (nanoparticles encapsulated with Cy5.5 labelled siPol2 and CG-CO₂) and Cy5.5-siPol2@NPs (without CO₂) (nanoparticles encapsulated with Cy5.5 labelled siPol2 and CG) for 1, 3, and 6 h at 37° C. The cell nuclei were stained using DAPI, the endo/lysosomes were stained using LysoTracker Green, and siPol2 were labelled with Cy5.5; DIC represents differential interference contrast. For the Cy5.5-siPol2@NPs (without CO₂) group, the green and red fluorescence overlaps at all the time points. For the Cy5.5-siPol2@NPs (with CO₂) treatment, the overlap is reduced at all the three time points and minimal at 6 h. Scale bars: 20 μm and 5 μm for low and high magnification images, respectively. The experiments were repeated three times independently. FIG. 3B shows quantitative analysis of co-localization of Cy5.5-siPol2 with endo/lysosomes labelled with LysoTracker Green. Manders' Coefficient M1 denotes the fraction of Cy5.5-siPol2 overlapping with LysoTracker Green, and M2 denotes the fraction of LysoTracker Green overlapping with Cy5.5-siPol2. The coefficients are close “1” if they are highly co-localized (n=10 images from three independent experiments). Error bars denote mean±s.d., *: p<0.05 and ***: p<0.001. The statistical significance was assessed by Student t-test (paired, two tailed) and one-way ANOVA with a Dunnett's host-hoc test for M1 and M2, respectively.

FIGS. 4A to 4G show nanoparticle mediated POLR2A inhibition selectively kills POLR2^{loss} cells. FIG. 4A is a schematic illustration of the strategy for killing TP53^{loss}/POLR2A^{loss} cells by POLR2A inhibition. POLR2A is almost always co-deleted with TP53. Moreover, POLR2A expression levels are significantly correlated to POLR2A gene copy number, inspiring the new strategy for treating TNBC. FIG. 4B shows wild type POLR2A^{1oss} MDA-MB-453 (TP53^{-/mut}, POLR2A^{-/+}) and POLR2A^{neutral} MDA-MB-231 (TP53^{+/mut}, POLR2A^{+/+}) breast cancer cells are treated with different dosages of siPol2-laden nanoparticles (siPol2@NPs), non-target siRNA-laden nanoparticles (siNT@NPs), or free siPol2 (f-siPol2) for 72 h. Colony formation assay with crystal violet staining and quantitative analyses of absorbance at 570 nm showing the cell viability are given on the left and right, respectively (n=3 independent experiments with 3 replicates in each experiment). FIG. 4C show viability of the two types of cells quantified using the cell counting kit (CCK-8) after the aforementioned treatments (n=3 independent experiments with 3 replicates in each experiment). FIG. 4D show protein levels of POLR2A in the two types of cells without or with various treatments. Without any treatment (i.e., the two control groups), POLR2A protein expression in POLR2A^{loss} MDA-MB-453 cells is lower than that in POLR2A^{neutral} MDA-MB-231 cells. FIG. 4E shows cell colony assay showing that the POLR2A^{loss} HCC1937 (Loss-1 and Loss-2) cells have similar proliferation to the parent HCC1937 POLR2A^{neutral} (Neutral) cells (n=3 independent experiments with 3 replicates in each experiment). FIG. 4F is a cell colony assay with Isogenic POLR2A^{neutral} and POLR2A^{loss} HCC1937 (TP53^{+/mut}) cells for confirming the observation that POLR2A^{loss} cells are highly sensitive to POLR2A inhibition using siPol2@NPs. CRISPR (clustered regularly interspaced short palindromic repeat)/Cas9 system was used to generate the isogenic HCC1937 cell lines with hemizygous loss of POLR2A. siPol2@NPs could significantly inhibit the proliferation of POLR2A^{loss} HCC1937 (loss-1 and loss-2) cells, but not of parent POLR2A^{neutral} HCC1937 cells (n=3 independent experiments with 3 replicates in each experiment). FIG. 4G shows protein levels of POLR2A in isogenic POLR2A^{loss} (Loss-1, Loss-2) and POLR2A^{neutral} (Neutral) HCC1937 cells without (Control) and with various treatments, showing the dose dependent inhibition of POLR2A with the siPol2@NPs. Error bars denote mean±s.e.m. The experiments in d and g were repeated three times independently.

FIGS. 5A to 5J show targeted POLR2A inhibition selectively suppresses the growth of isogenic cells derived POLR2A^{loss} tumours. FIG. 5A shows in vivo whole animal imaging (both front and back) of Cy5.5 fluorescence at pre-injection, and 2 and 8 h after intravenous injection of saline, Cy5.5-siPol2, and Cy5.5-siPol2@NPs. The experiments were repeated three times independently. The scheme indicates the locations of tumours (the 4^{th} inguinal mammary fat pads on both left and right sides) in mice. FIG. 5B shows ex vivo imaging of Cy5.5 fluorescence in tumours together with four critical organs collected after in vivo imaging at 8 h. Tumour-L and Tumour-R denote tumours on the left and right of the mouse, respectively. The experiments were repeated three times independently. FIG. 5C shows an illustration of the treatment intervals. The mice were injected with the various treatments twice a week. FIG. 5D to 5F show tumour growth (FIG. 5D), weight (FIG. 5E), and gross images (FIG. 5F) of tumours derived from isogenic POLR2A^{loss} and parent POLR2A^{neutral} HCC1937 (TP53^{+/mut}, POLR2A^{+/+}) cancer cells with various treatments. The data indicate that tumours with hemizygous loss of POLR2A are highly sensitive and vulnerable to further POLR2A inhibition. Error bars denote mean±s.d. In d, the p values for comparison of siPol2@NPs versus Saline, siPol2@NPs versus f-siPol2, and siPol2@NPs versus f-siPol2 are 0.0099 (indicated on FIG. 5D), 0.0086, and 0.0049, respectively. Statistical significance was assessed by one-way ANOVA with a Fisher's LSD post hoc test (FIG. 5D) or Dunnett's post hoc test (FIG. 5E). The biologically independent sample size n=7. Scale bar: 2 cm. FIGS. 5G and 5H show immunofluorescence staining (FIG. 5G) of POLR2A's tumours and quantification of POLR2A expression (FIG. 5H) in tumours with various treatments (n=3 independent experiments with 3 replicates in each experiment). The statistical significance was assessed by one-way ANOVA with a Dunnett's post hoc test. FIGS. 5I and 5J show protein level of POLR2A (FIG. 5I) and H&E staining (FIG. 5J) of aforementioned POLR2A^{loss} tumours. The experiments were repeated three times independently. In FIG. 5I contains two representative samples for each treatment are shown. Error bars denote mean±s.d. Scale bar: 50 μm; **: p<0.01; and ***: p<0.001.

FIGS. 6A to 6I show targeted POLR2A inhibition selectively suppresses the growth of wild type cells derived POLR2A^{loss} tumours. FIG. 6A is a schematic illustration of the tumours established by implanting MDA-MB-453 (TP53mut, POLR2A^{loss}) cells on the left and MDA-MB-231 (TP53^{mut}, POLR2A^{neutral}) cells on the right 4^{th} inguinal mammary fat pads. FIG. 6B is an illustration of the treatment intervals. The mice were injected with the various treatments twice a week. FIGS. 6C to 6E contains growth curves (FIG. 6C), weight (FIG. 6D), and gross images (FIG. 6E) of tumours derived from POLR2A^{loss} and POLR2A^{neutral} human TNBC cells with various treatments. The data indicate that tumours with POLR2A^{loss} are highly sensitive and vulnerable to further POLR2A inhibition. Error bars denote mean±s.d. In FIG. 6C, the p values for comparisons of siPol2@NPs versus Saline and siPol2@NPs versus f-siPol2 are 0.0114 (indicated in FIG. 6C) and 0.0061, respectively. The statistical significance was assessed by one-way ANOVA with a Fisher's LSD post hoc test for FIG. 6C or Dunnett's post hoc test for FIG. 6D. The biologically independent sample size n=6. Scale bar: 2 cm. FIG. 6F to 6G show immunofluorescence staining (FIG. 6F) of POLR2A^{loss} tumours and quantitative data of POLR2A expression (FIG. 6G) in tumours with various treatments (n=3 independent experiments with 3 replicates in each experiment). The statistical significance was assessed by one-way ANOVA with a Dunnett's post hoc test. h-i, Protein level of POLR2A (FIG. 6H) and H&E staining (FIG. 6I) of the aforementioned POLR2A^{loss} tumours. The experiments were repeated three times independently. FIG. 6H shows two representative samples for each treatment. Scale bar: 50 μm. Error bars denote mean±s.d.; *: p<0.05; and ***, p<0.001.

FIGS. 7A to 7D show colateral deletion of POLR2A with TP53 in triple negative breast cancers. FIG. 7A shows concomitant deletion of POLR2A in human TNBC samples (TOGA, Origin: Cell 2015) containing hemizygous loss of TP53 (n=81 biologically independent samples). FIGS. 7B to 7D show the correlation of POLR2A mRNA (FIG. 7B), TP53 mRNA (FIG. 7C), or p53 protein (FIG. 7D) expression with copy number variation in TNBC samples. TOGA: The Caner Genome Atlas, and TNBC: triple negative breast cancer. The statistical significance was assessed by Student's t-test (unpaired, two-tailed). The Box-Whisker plots present a five-number summary: minima, lower quartile, centre, upper quartile, and maxima. **, p<0.01.

FIGS. 8A and 8B show characterization of the modification of chitosan with guanidine. FIG. 8A shows UV-vis absorbance of chitosan before and after modification with guanidine. The specific absorption peak at 245 nm of guanidine in chitosan-guanidinate indicates the successful modification of guanidine onto chitosan. FIG. 8B shows FTIR spectra of chitosan and chitosan-guanidinate showing the absorption of guanidine group at 1594 cm⁻¹ and 1620 cm⁻¹. UV-vis: ultraviolet-visible light, and FTIR: Fourier transform infrared spectroscopy. The experiments were repeated three times independently.

FIG. 9 shows zeta potential of siPol2-laden nanoparticles. Zeta potential (−22.4±2.1 mV, mean±s.d.) of the siPol2-laden nanoparticles (siPol2@NPs) in deionized water (n=3 independent experiments).

FIGS. 10A and 10B show nanoparticles synthesized using chitosan-guanidinate without CO₂ are not pH responsive. FIG. 10A shows TEM images of the nanoparticles under different pH conditions showing that the nanoparticles could maintain their intact spherical morphology and core-shell structure and are not responsive to low pH treatments (pH 6.0 and pH 5.0). FIG. 10B shows nanoparticle size distribution determined by dynamic light scattering (DLS) at pH 7.4, 6.0, and 5.0. Scale bars in a: 500 nm for top row and 100 nm for bottom row. The experiments were repeated three times independently.

FIG. 11 shows electrophoretic stability assay of free siPOLR2A. The siRNA was incubated in serum at 37° C. for up to 60 min. The data indicate most of the free siRNA degraded in serum in less than 10 minutes. The experiments were repeated three times independently.

FIGS. 12A to 12C show generating isogenic HCC1937 cell lines bearing hemizygous loss of POLR2A. FIG. 12A is a schematic illustration of the Cas9/sgRNA-targeting sites in the POLR2A gene. FIG. 12B contains sequences of mutant POLR2A alleles in the isogenic colonies. Protospacer adjacent motif (PAM) sequences are highlighted in red. Small deletions in the targeted region lead to an open reading frame shift, producing only a short stretch of the amino-terminal peptide without any functional domains of POLR2A. FIG. 12C shows protein levels of POLR2A in POLR2A^{neutral} and POLR2A^{loss} HCC1937 cells. The experiments were repeated three times independently.

FIG. 13 shows whole body and organ biodistribution of nanoparticles. Top: in vivo whole animal imaging (both front and back) of Cy5.5 fluorescence at pre-injection, and 2 and 8 h after intravenous injection of saline, free Cy5.5-siPol2, and Cy5.5-siPol2@NPs. Three mice were used for each of the three experimental conditions. Bottom: ex vivo imaging of Cy5.5 fluorescence in tumours together with four critical organs collected after in vivo imaging at 8 h. Tumour-L and Tumour-R denote tumour on the left and right sides of the mice, respectively (n=3 biologically independent samples).

FIGS. 14A and 14B shows no evident systemic toxicity for the nanoparticle-mediated delivery of siPol2 to isogenic tumours. FIG. 14A shows body weight of mice with various treatments showing no significant difference between the different treatments. Tumours were established by implanting isogenic HCC1937 (POLR2A^{loss}) cells on the left and parent HCC1937 (POLR2A^{neutral}) cells on the right. Error bars represent s.d. (n=7 biologically independent samples). FIG. 14B contains representative H&E-stained slices of major organs in mice treated with siPol2@NPs or saline collected on day 30. The experiments were repeated three times independently. Scale bar: 100 μm.

FIG. 15 shows negligible liver toxicity and immune response induction by nanoparticles. No significant difference was observed for the two liver enzymes:aspartate aminotransferase (AST) and alanine aminotransferase (ALT). Significantly increased levels of IFN-γ and MCP-1 were observed only for the siNT@NP and siPol2@NP treatments at 6 h after injection, which returned to the baseline levels on day 1 and thereafter. TNF-α:tumour necrosis factor-α, IFN-γ: interferon-γ, IL-6: interleukin-6, IL-10: interleukin-10, and MCP-1: monocyte chemoattractant protein-1. The biologically independent sample size n=4. Error bars denote mean±s.d., ***: p<0.0001. The statistical significance was assessed by one-way ANOVA with a Dunnett's post-hoc test.

FIGS. 16A and 16B show no evident systemic toxicity for the nanoparticle-mediated delivery of siPol2 to wild type cells derived tumours. FIG. 16A shows body weight of mice with various treatments showing no significant difference between the different treatments. Tumours were established by implanting MDA-MB-453 (POLR2A^{loss}) cells on the left and MDA-MB-231 (POLR2A^{neutral}) cells on the right. Error bars represent s.d. (n=6 biologically independent samples). FIG. 16B contains representative hematoxylin & eosin (H&E)-stained slices of major organs in mice treated with siPol2@NPs or saline collected on day 30. The experiments were repeated three times independently. Scale bar: 100 μm.

FIGS. 17A and 17B show POLR2A^{loss} cells are highly sensitive to POLR2A inhibition regardless of their TP53 status. FIG. 17A shows protein level of POLR2A and p53 of four different isogenic HER18 HER2+ breast cancer cell lines. The experiments were repeated three times independently. FIG. 17B show two POLR2A's HER18 (TP53^{+/+}, POLR2A^{+/-} and TP53^{+/-}, POLR2A^{+/-}) cell lines and two POLR2A^{neutral} HER18 (TP53^{+/+}, POLR2A^{+/+} and TP53^{+/-}, POLR2A^{+/+}) cell lines are treated with different dosages of siPol2 in the form of siPol2@NPs for 72 h. Quantitative analyses of the cell viability using crystal violet staining are shown (n=3 independent experiments with 3 replicates in each experiment). Error bars denote mean±s.e.m.

FIGS. 18A to 18H show targeted POLR2A inhibition for treating HER2+ breast cancer. FIG. 18A is a schematic illustration of the tumours established by implanting isogenic HER18 (TP53^{+/+}, POLR2A^{-/+}, or POLR2A^{loss}) cells on the left and parent HER18 (TP53^{+/+}, POLR2A^{+/+}, or POLR2A^{neutral}) cells on the right 4th inguinal mammary fat pads. FIG. 18B is an illustration of the treatment intervals. The mice were injected with the various treatments twice a week. FIGS. 18C to 18E show tumour growth (FIG. 18C), weight (FIG. 18D), and gross images (FIG. 18E) of tumours derived from isogenic POLR2A^{loss} and parent POLR2A^{neutral} HER18 cancer cells with various treatments. The data indicate that tumours with hemizygous loss of POLR2A are highly sensitive and vulnerable to further POLR2A inhibition. Error bars denote mean±s.d. In FIG. 18C, the p values for comparisons of siPol2@NPs versus Saline, siPol2@NPs versus f-siPol2, and siPol2@NPs versus f-siPol2 are 0.0166 (indicated in FIG. 18C), 0.0063, and 0.0142, respectively. The statistical significance was assessed by one-way ANOVA with a Fisher's LSD post hoc test for FIG. 18C or Dunnett's post hoc test for FIG. 18D. The biologically independent sample size n=6. Scale bar: 2 cm. FIGS. 16F and 16G show H&E staining (FIG. 16F) and immunofluorescence staining (FIG. 16G) of POLR2A^{loss} tumours from the four different treatments. The experiments in FIG. 16F were repeated three times independently. FIGS. 16H and 16I show quantitative (FIG. 16H) and qualitative (FIG. 16I) data of POLR2A expression in the aforementioned POLR2A^{loss} tumours with various treatments (n=6 biologically independent samples). In FIG. 16H, the statistical significance was assessed by one-way ANOVA with a Dunnett's post hoc test. In FIG. 16I, two representative samples for each treatment are shown. Error bars denote mean±s.d., *: p<0.05; and ***: p<0.001. Scale bars in FIGS. 16F and 16G: 50 μm.

FIGS. 19A to 19E show association between gene amplification, gene expression and HER2+ breast cancer on chromosome 17. FIG. 19A is a heatmap of genomic segment copy-number abnormalities (log-ratio measurements) of human chromosome 17 in 1080 breast invasive carcinomas. Positive log-ratios indicate degree of copy number gain (red) whereas negative values mark the loss (blue). FIG. 16B shows co-amplification of WIP1 and genes from 17q22 to 17q23. FIG. 16C shows HER2+ subtype significantly enriched in breast cancers harboring WIP1-contacting 17q23 amplicon. FIG. 16D shows correlation between gene expression aberration and copy number variation for genes, WIP1, MIR21, and HER2 (ERBB2) in breast tumors. FIG. 16E contains soft agar colony formation assays with MMTV-ERBB2 mouse mammary epithelial cells transduced with control vector or lentiviral vector expressing the indicated genes.

FIGS. 20A to 20D show suppression of miR-21 and WIP1 inhibits proliferation and tumorigenic potential of HER2+ breast cancer cells. FIG. 20A shows Kaplan-Meier analysis of tumor-free survival in female MMTV-ErbB2 wild type (n=12), MMTV-ErbB2; WIP1^{-/-} (n=12) and MMTV-ErbB2; MIR21^{-/-} (n=13) mice. FIG. 12B shows amplification of MIR21 and WIP1 is associated with poor overall survival in patients with HER2+ breast cancer, but not with patients with luminal A, luminal B or basal-like breast cancer. FIG. 20C shows cell growth curve of H605 cells (MMTV-ERBB2 tumor cells) expressing Dox-inducible control shRNA or specific shRNA targeting WIP1 or MIR21. FIG. 20D shows mammosphere formation assay in H605 cells expressing Dox-inducible control shRNA, specific shRNA targeting WIP1 or MIR21. Right panel demonstrates the quantitative data using Image J software.

FIGS. 21A to 21C show overexpression of miR-21 and WIP1 promotes oncogenic transformation of HER2+ breast cancer cells. FIG. 21A shows relative expression levels of the predicted miR-21 targets in primary mammary epithelial cells isolated from MMTV-ErbB2 wild-type or MIR21−/− virgin females at the age of 8-9 weeks. Data represents the mean expression levels normalized to the endogenous snoRNA55 control from three independent experiments. FIG. 21B shows miR-21 targets are enriched in pathways associated with cell proliferation, survival and metastasis in mammary cells. FIG. 21C shows mammary epithelial cells derived from MMTV-ErbB2 mouse were transduced with lentivirus expressing miR-21 and/or WIP1. Upper panels: representative photomicrographs of SA-β-galactosidase (SA-13-Gal) staining observed in bright field. Bottom panels: miR-21 and WIP1 expression levels as determined by q-PCR, and percentages of SA-β-Gal-positive cells were calculated. Error bars represent mean±SD of triplicate experiments.

FIGS. 22A to 22D show DDX5 gene is co-amplified with MIR21 and DDX5 facilitates maturation of pri-miR-21. FIG. 22A shows immunoprecipitation (IP) and western blotting analyses were performed using indicated antibodies. Normal immunoglobulin G (IgG) was used as a negative control for IP, The RNA-binding proteins DDX1 was used as a positive control for the Drosha-binding proteins. FIG. 22B shows the DDX5-bound pri-miRNAs immunoprecipitated with DDX5 and analyzed by qRT-PCR. Control IgG was used as a negative control. FIG. 22C shows levels of primary or mature forms of the miR-21 were analyzed in control and DDX5-knockdown breast cancer cells harboring 17q123 amplicon. FIG. 22D shows levels of mature miR-21 and DDX5 were analyzed in breast tumor samples using in situ hybridization and immunohistochemistry. Representative staining images of tissue samples are shown. Scale bar: 100 μm. ** p<0.05, ***p<0.001.

FIGS. 23A to 23D show inhibition of miR-21 and WIP1 kills HER2+ breast cancer cells harboring 17q23 amplicon. FIGS. 23A to 23C show HER18 (FIG. 23A), BT-474 (FIG. 23B) or MDA-MB-453 (FIG. 23C) cells were incubated with or without the WIP1 inhibitor GSK2830371 at the indicated concentrations for 72 h. The cell viability was then measured and the results are presented as % vehicle±S.D. Protein levels are shown at the bottom of each panel by immunoblotting. FIGS. 23D1 and 23E1 show HER18 (FIG. 23D1) or BT-474 (FIG. 23E1) cells with or without Dox-induce miR-21 knockdown were incubated indicated concentrations of GSK2830371 for 72 h. The cell viability was then measured and the results are presented as % vehicle±S.D. FIGS. 23D2 and 23E2 show after treatment the cell lysates were subjected to Western blot analyses with the indicated antibodies. FIGS. 23D3 and 23E3 show knockdown efficiency of miR-21 measured by luciferase reporter assay.

FIGS. 24A to 24G show inhibition of miR-21 and WIP1 sensitizes HER2+ breast cancer cells to the treatment of trastuzumab. FIGS. 24A and 24B show parental or trastuzumab-resistant HER2+ breast cancer cells (HER18 or BT-474) were incubated with trastuzumab at the indicated concentrations for 72 h. The cell viability was then measured and the results are presented as % vehicle±S.D. Right: the cell lysates were subjected to Western blot analyses with the indicated antibodies. FIGS. 24C and 24D show HER18R (FIG. 24C) or BT-474R (FIG. 24D) cells with or without Dox-induce miR-21 knockdown were incubated indicated concentrations of GSK2830371 for 72 h. The cell viability was then measured and the results are presented as % vehicle±S.D. Right: the cell lysates were subjected to Western blot analyses with the indicated antibodies. FIG. 24E shows HER18R cells with or without Dox-induce miR-21 knockdown were incubated with GSK2830371 (0.2 μM) and/or trastuzumab (1 μg/ml) for 72 h. The cell viability was then measured and the results are presented as % vehicle±S.D. FIGS. 24F and 24G show gross tumor images (FIG. 24F) and tumor growth curves (FIG. 24G) of xenograft tumors derived from orthotopically implanted parental or trastuzumab-resistant HER18 cells expressing Dox-inducible WIP1 or DDX5 shRNA or anti-miR21 oligonucleotides with trastuzumab treatment (5 mg/kg, twice per week).

FIGS. 25A to 25D show synthesis of nanoparticles for drug delivery. FIG. 25A shows nanoparticles encapsulating therapeutic agent(s) were synthesized using a double-emulsion water-in-oil-in-water method. The inner water phase containing anti-miR21 inhibitor and CG-CO₂ were encapsulated in the core, and hydrophobic WIP1 inhibitor (GSK2830371) together with PLGA and DPPC dissolved in oil phase was used to form the shell structure. FIG. 24B contains TEM images of nanoparticles in pH 7.4, 6.0, and 5.0, respectively. Nanoparticles maintained spherical morphology with clear core-shell structure in pH 7.4, and enlarged and broken under low pH (pH 6.0 and pH 5.0) indicated the pH-responsive behavior. FIG. 25C shows electrophoretic stability assay of in-MW@NP and free anti-miR21 inhibitor at different time points incubation in serum at 37° C. The observable signal of in-MW@NP indicated that the nanoparticle encapsulation could protect anti-miR21 inhibitor from degradation in serum up to 36 hours. FIG. 25D shows typical confocal imaging of cells incubated with Dex-Rho@NP for 1-6 h. The change of the fluorescence overlap of Dex-Rho and Lysotracker (green) shows the CO₂-associated pH-responsive endo/lysosomal escape in HER18R cells. Cell nuclei were stained with DAPI, endo/lysosomal vesicles were stained with Lyso Tracker Green, and DIC represented differential interference contrast.

FIGS. 26A to 26G show in vivo efficacy of nanoparticle-encapsulated WIP1 and miR-21 inhibitors in trastuzumab-resistant breast tumor models. FIG. 26A shows relative cell viability of trastuzumab-resistant HER18R treated with indicated doses of nanoparticles encapsulating WIP1 or miR-21 inhibitors for 72 h. FIG. 26B shows in vivo whole animal imaging of ICG fluorescence at 1 h, 6 h, and 24 h, respectively, after intravenous injection of saline, free ICG and NP-ICG nanoparticles. Ex vivo imaging of ICG fluorescence of tumor together with five important organs collected when the mice were sacrificed at 24 h post-injection. The arrows indicate the locations of tumors in mice. FIGS. 26C to 26E show gross tumor images (FIG. 26C), tumor growth curves (FIG. 26D) and tumor weight (FIG. 26E) of xenograft tumors derived from orthotopically implanted trastuzumab-resistant HER18R cells. Once tumors were palpable, mice were randomly divided to 4 groups and then treated with either control, WIP1 and/or miR-21 inhibitor nanoparticles (twice per week) intraperitoneally. (FIG. 26F, 26G) Quantification of cell proliferation (Ki-67 staining, FIG. 26F) and apoptosis (cleaved caspase-3 staining, FIG. 26G) in the xenografted tumor tissues described above.

FIGS. 27A to 27C show co-amplification and overexpression of WIP1 and MIR21 in the 17q23 amplicon of HER2+ breast cancer cell lines. FIGS. 27A and 27B show copy numbers (FIG. 27A) and relative RNA expression levels (FIG. 27B) of HER2, WIP1 and MIR21 in a panel of breast cancer cell lines. The non-tumorigenic epithelial cell line MCF10A was used as a control. FIG. 27C shows protein expression levels of HER2 and WIP1 in a panel of breast cancer cell lines as determined by Western blot.

FIGS. 28A to 28D show DDX5 gene is co-amplified with MIR21 and its expression facilitates maturation of pri-miR-21. FIG. 28A shows DDX5 interacts with pri-miR-21 in the microprocessor. The assay is based on the addition of a specific bacteriophage MS2 RNA hairpin loop sequence to pri-miR-21, followed by co-expression of the MS2-tagged RNA together with GST-tagged MS2P that specifically binds MS2. The pri-miR-21 binding proteins were analyzed by gel electrophoresis and the protein bands of interest were determined by mass spectrometry. FIG. 28B shows the levels of DDX1 and DDX5 measured by qPCR in control and knockdown MCF-7 cells. Student t-test, *** p<0.001. FIG. 28C shows a box-and-whisker plot used to visualize DDX5 expression levels in breast tumors. The Shapiro-Wilk test was applied to verify that mRNA expression does not follow a normal distribution in each group. FIG. 28D shows miR-21 levels are positively correlated with DDX5 expression levels in breast cancer. Intensity of miR-21 and DDX5 signals were scored in breast adenocarcinomas using tumor microarray and Pearson's correlation coefficient was analyzed by GraphPad Prism 6 software. Scoring=intensity (1, 2 or 3)×proportion (10-100%).

FIGS. 29A to 29D show inhibition of miR-21 sensitizes HER2+ breast cancer cells to the treatment of trastuzumab. FIG. 29A shows knockdown efficiency of miR-21 in Trastuzumab-resistant HER18R cells was measured by luciferase reporter assay. The luciferase activity of the reporter gene with miR-21-targeting 3′-UTR is negatively correlated with the level of miR-21. FIG. 29B shows Western blot analyses were performed to show knockdown efficiency of WIP1 (left) or DDX5 (right) in the HER18R cells. FIG. 29C shows tumor weight of xenograft tumors derived from orthotopicaly implanted isogenic parental or trastuzumab-resistant HER18 cells expressing Dox-inducible WIP1 or DDX5 shRNA or anti-miR21 oligonucleotides with trastuzumab treatment (5 mg/kg, twice per week). FIG. 29D shows quantification of knockdown efficiencies of miR-21, WIP1 or DDX5 in the xenografted tumor tissues described above.

FIGS. 30A and 30B shows synthesis of nanoparticles for delivering WIP1 and miR-21 inhibitors. FIG. 30A shows nanoparticle size distribution determined by dynamic light scattering (DLS) in pH 7.4, pH 6.0, and pH 5.0, respectively. FIG. 30B shows surface zeta potential of in-MW@NP nanoparticles

FIGS. 31A and 31B show in vivo efficacy of nanoparticle-encapsulated WIP1 and miR-21 inhibitors in trastuzumab-resistant breast tumor models. FIG. 31A contains representative images of H&E, Ki-67 (cell proliferation) and cleaved caspase-3 (apoptosis) staining in orthotopically implanted HER18R tumor tissues. Scale bar: 100 μm. FIG. 31B shows changes in the body weights of NU/J mice with treatments as described in the HER18R groups.

FIGS. 32A to 32C show synthesis of the dual-targeting RP@NB-HF nanobomb (NB). FIG. 32A is a schematic illustration of the nanobomb synthesis with a double emulsion method. FIG. 32B shows the mechanism of the low pH-triggered “bomb” effect. FIG. 32C shows the bomb effect could damage the nanoparticle morphology at low pH. HA (H) is for CSC targeting and FCD (F) is for active tumor targeting.

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of chemistry, biology, and the like, which are within the skill of the art.

The following examples are put forth so as to provide those of ordinary skill in the art with a complete disclosure and description of how to perform the methods and use the probes disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

Before the embodiments of the present disclosure are described in detail, it is to be understood that, unless otherwise indicated, the present disclosure is not limited to particular materials, reagents, reaction materials, manufacturing processes, or the like, as such can vary. It is also to be understood that the terminology used herein is for purposes of describing particular embodiments only, and is not intended to be limiting. It is also possible in the present disclosure that steps can be executed in different sequence where this is logically possible.

It must be noted that, as used in the specification and the appended claims, the singular forms “a,” “an,” and “the” include plural referents unless the context clearly dictates otherwise.

### Nanoparticles

Disclosed herein is a pH activated nanoparticle that contains a shell and core, wherein the shell comprising a phospholipid bilayer and the core comprising a gas bound to a substrate by a pH sensitive interaction.

In some embodiments, the nanoparticle composition further comprises at least one targeting agent, wherein the targeting agent selectively targets the nanoparticle to diseased tissue/cells, thereby minimizing whole body dose. In some embodiments, the nanoparticle composition further comprises at least one targeting agent, wherein the targeting agent comprises an antibody or functional fragment thereof, a small molecule, a peptide, a carbohydrate, a siRNA, a microRNA, a protein, a nucleic acid, an aptamer, a second nanoparticle, a cytokine, a chemokine, a lymphokine, a receptor, a lipid, a lectin, a ferrous metal, a magnetic particle, a linker, an isotope and combinations thereof.

In some embodiments, the nanoparticles of the nanoparticle composition have a size of about 10 nm to about 200 nm. In some embodiments, a drug load in the nanoparticle composition is about 0.1% to about 90% by weight of the composition.

### Core

In some embodiments, the core comprises a carrier for the pH sensitive agent. Chitosan-based carriers have become one of the major non-viral vectors that have received increasing interest as a reliable gene or siRNA delivery system. Chitosan has low toxicity, low immunogenicity, excellent biocompatibility (Shu & Zhu (2002) Eur. J. Pharm. Biopharm. 54: 235-243; Lee et al., (2005) Biomaterials 26: 2147-2156). Due to its positive charge, it can easily form polyelectrolyte complexes with negatively charged nucleotides by electrostatic interaction.

Chitosan is obtained by deacetylation of chitin, which is the biodegradable polysaccharide consisting of repeating D-glucosamine and N-acetyl-D-glucosamine units, linked via (1-4) glycosidic bonds. Chitosan is almost non-toxic in animals (Rao & Sharma (1997) Biomed. Mater. Res. 34: 21-28) and humans (Aspden et al., J. Pharm. Sci. 86 (1997) 509-513), with an LD50 in rats of 16 g/kg (Chandy & Sharma (1990) Biomater Artif Cells Artif Organs 18: 1-24). Chitosan can be characterized by several physicochemical properties, including molecular weight, degree of deacetylation, viscosity, and crystallinity (Kas H. S. (1997) J. Microencapsul. 14: 689-711). The desirability of chitosan as a gene delivery carrier is based on its cationic property to allow binding of negatively charged siRNA via electrostatic interactions.

Therefore, in some embodiments, the core substrate for binding the gas is a modified chitosan. For example, in some embodiments, the core substrate comprises chitosan-guanidine (CG) or chitosan-arginine (CA).

In some embodiments, the core substrate comprises metformin or calcium carbonate.

In some embodiments, the gas comprises carbon dioxide, ammonia, nitric oxide, oxygen, or hydrogen gas.

Shell

In some embodiments, the shell of the nanoparticle comprises a phospholipid bilayer. Phospholipid head groups commonly found in nature generally contain phosphatidylcholine (PC), phosphatidylethanolamine (PE), phosphatidylinositol (PI), and phosphatidylserine (PS). Such phospholipids may be found in soybeans or egg yolks, though neither of these sources is commonly used in human clinical applications due to stability and contamination issues. Examples include Soybean phosphatidylcholine (SPC), hydrogenated soybean phosphatidylcholine (HSPC), egg sphingomyelin (ESM), and egg phosphatidylcholine (EPC). Synthetic phospholipid derivatives may include, but are not limited to: dipalmitoyl phosphatidylglycerol (DPPG), dimyristoyl phosphatidylglycerol (DMPG), dioleoyl phosphatidylglycerol (DOPG), distearoyl phosphatidylglycerol (DSPG), dipalmitoylphosphatidylcholine (DPPC), distearoyl phosphatidylcholine (DSPC), dimyristoyl phosphatidylcholine (DMPC), hydrogenated soy phosphatidylcholine (HSPC), dioleoyl phosphatidylcholine (DOPC), phosphatidylethanolamines, including 1,2-distearoyl-sn-glycero-3-phosphoethanolamine (DSPE), dimyristoyl phosphatidylethanolamine (DMPE), dipalmitoyl phosphatidylethanolamine (DPPE), dioleoyl phosphatidylethanolamine (DOPE), dimyristoyl phosphatidylserine (DMPS), dipalmitoyl phosphatidylserine (DPPS), and dioleoyl phosphatidylserine (DOPS).

In some embodiments, the shell comprises at least one of poly(lactic-co-glycolic acid) (PLGA) or its PEGylated form PEG-PLGA, polylactic acid (PLA) or its PEGylated form PEG-PLA, polyglycolic acid (PGA) or its PEGylated form PEG-PGA, poly-L-lactide-co-ε-caprolactone (PLCL) or its PEGylated form PEG-PLCL, Hyaluronic acid (HA), polyacrylic acid (PAA) or PEG-PAA, polyphosphate (polyP), poly(acrylic acid-co-maleic acid), poly(butylene succinate), poly(alkyl cyanoacrylate) (PAC) or its PEGylated form PEG-PAC or combinations thereof.

In some embodiments, the shell comprises a poloxamer (Pluronic®). Poloxamers are tri-block copolymers of poly(ethylene oxide) poly(propylene oxide)-poly(ethylene oxide) (PEO-PPO-PEO). This group of synthetic polymers is thermoreversible in aqueous solutions. The sol-gel transition is governed by the composition, molecular weight, and concentration of each constituent block polymer. The hydrophilic ethylene oxide and the hydrophobic propylene oxide give poloxamers an amphiphilic structure—meaning it has a polar, water-soluble group attached to a nonpolar water-insoluble hydrocarbon chain. Amphiphilic block copolymer molecules self-assemble into micelles (a packed chain of molecules) in aqueous solution. Micelle formation is temperature dependent and affects the degradation properties of the biomaterial: below a certain characteristic temperature known as the critical micelle temperature, both the ethylene and propylene oxide blocks are hydrated and the PPO block becomes soluble. Because the lengths of the polymer blocks can be customized, many different poloxamers exist that have slightly different properties. For the generic term “poloxamer”, these copolymers are commonly named with the letter “P” (for poloxamer) followed by three digits, the first two digits×100 give the approximate molecular mass of the polyoxypropylene core, and the last digit×10 gives the percentage polyoxyethylene content (e.g., P407=Poloxamer with a polyoxypropylene molecular mass of 4,000 g/mol and a 70% polyoxyethylene content). For the Pluronic® tradename, coding of these copolymers starts with a letter to define its physical form at room temperature (L=liquid, P=paste, F=flake (solid)) followed by two or three digits, The first digit (two digits in a three-digit number) in the numerical designation, multiplied by 300, indicates the approximate molecular weight of the hydrophobe; and the last digit×10 gives the percentage polyoxyethylene content (e.g., L61=Pluronic with a polyoxypropylene molecular mass of 1,800 g/mol and a 10% polyoxyethylene content). Poloxamers are commercially available, and methods for their synthesis are known to those of skill in the art.

In some embodiments, the phospholipids present in the nanoparticle composition is 1,2-dipalmitoyl-sn-glycero-3-phosphocholine (DPPC) and 1,2-dipalmitoyl-sn-glycero-3-phosphoethanolamine (DPPE-PEG). In some embodiments, the phospholipids present in the nanoparticle composition is L-α-phosphatidylcholine (L-α-PC) and 1,2-dipalmitoyl-sn-glycero-3-phosphoethanolamine (DPPE-PEG). In some embodiments, the one or more phospholipids present in the nanoparticle composition is 1,2-dioleoyl-3-trimethylammonium-propane (DOTAP).

In some embodiments, the phospholipids present in the nanoparticle composition is 1,2-dioleoyl-3-trimethylammonium-propane (DOTAP) and the polymer is polyphosphate (polyP). In some embodiments, the one or more phospholipids present in the nanoparticle composition is 1,2-dioleoyl-3-trimethylammonium-propane (DOTAP) and the polymer is PEG polyacrylic acid (PAA).

In some embodiments, a molar ratio of the lipid(s) to a PEGylated lipid(s) in the nanoparticle composition is about 100:0 to about 50:50. In some embodiments, a molar ratio of a saturated lipid(s) to an unsaturated lipid(s) in the nanoparticle composition is about 100:0 to about 25:75. In some embodiments, a molar ratio of capecitabine to lipid(s) in the nanoparticle composition is about 90:10 to about 10:90. In some embodiments, a molar ratio of lipid(s) to polymer in the nanoparticle composition is about 100:0 to about 10:80. In some embodiments, a molar ratio of capecitabine to polymer in the nanoparticle composition is about 100:0 to about 10:90. In some embodiments, the nanoparticle composition exhibits a zeta potential of from about −80 mV to about 80 mV.

### PH-Sensitive Agents

In some embodiments, the core further comprises a pH sensitive therapeutic or diagnostic agent. The disclosed nanoparticle assists in endosomal escape of the pH sensitive agent so it can reach the cytoplasm with minimal degradation.

In some embodiments, the pH sensitive therapeutic or diagnostic agent is an RNA or DNA oligonucleotide. For example, the oligonucleotides can be an mRNA, siRNA, miRNA, shRNA, or antisense oligonucleotide. In some embodiments, the RNA is a short or long noncoding RNA (ncRNA). In some embodiments, the therapeutic or diagnostic agent is a peptide or protein. In some embodiments, the therapeutic or diagnostic agent is an aptamer, such as a DNA, RNA or peptide aptamer. In some embodiments, the therapeutic or diagnostic agent is a labile small molecule.

### Methods for Delivering pH Sensitive Cargo

Disclosed herein is a method for delivering a pH sensitive cargo to the cytoplasm of a cell that involves loading the pH sensitive cargo into the disclosed pH activated nanoparticle and contacting the cell with the loaded nanoparticle.

### Methods of Treating TNBC

Also disclosed herein is a method for treating triple negative breast cancer (TNBC), such as TNBC with mutated or deleted TP53, in a subject that involves administering to the subject a therapeutically effective amount of the pH activated nanoparticle disclosed herein loaded with a POLR2A inhibitor. In some embodiments, the POLR2A inhibitor is an siRNA (siPol2). POLR2A siRNA are commercially available (e.g., Millipore Sigma, ThermoFisher Scientific) and can be designed using routine methods from the POLR2A gene sequence. In some embodiments, the siPol2 has the nucleic acid sequence CCAACAUGCUGACAGAUAU (SEQ ID NO:1), AUAUCUGUCAGCAUGUUGG (SEQ ID NO:2), CCAAGAAGCGGCUCACACA (SEQ ID NO:3), or UGUGUGAGCCGCUUCUUGG (SEQ ID NO:4).

### Methods of Treating HER2+ Breast Cancer

Also disclosed herein is a method for treating HER2+ breast cancer in a subject that involves administering to the subject a therapeutically effective amount of an miR-21 inhibitor and a WIP1 inhibitor. In some embodiments, the HER2+ breast cancer is trastuzumab-resistant and the method provides an alternative or addition to trastuzumab therapy. In some embodiments, the miR-21 inhibitor and a WIP1 inhibitor are loaded in a pH activated nanoparticle disclosed herein.

WIP1 Inhibitor

In some embodiments, the WIP1 inhibitor is a small molecule inhibitor. For example, the WIP1 inhibitor can be GSK2830371, shown below:

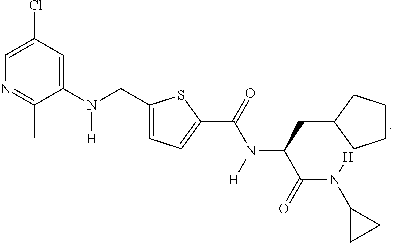 [SMILES: Cc1ncc(Cl)cc1NCc1ccc(C(=O)N[C@@H](CC2CCCC2)C(=O)NC2CC2)s1]

The wild-type p53-induced phosphatase Wip1, also known as protein phosphatase magnesium-dependent 1 delta (PPM1D) or PP2Cdelta, modulates cell cycling and may contribute to some forms of cancer. GSK2830371 is a potent inhibitor of Wip1 (IC₅₀=6 nM). It displays selectivity for Wip1 over 21 other phosphatases. GSK2830371 increases phosphorylation of Wip1 substrates and blocks cell cycling in hematopoietic cancer cells and in Wip1-amplified cancer cells with wild-type p53.

MIR-21 Inhibitor

In some embodiments, the miR-21 inhibitor is an anti-miR-21 oligonucleotide. In some embodiments, the anti-miR-21 oligonucleotide has the nucleic acid sequence

(SEQ ID NO: 5)
UCAACAUCAGUCUGAUAAGCUAG.

The disclosed miRNA antagonists are single-stranded, double stranded, partially double stranded or hairpin structured oligonucleotides that include a nucleotide sequence sufficiently complementary to hybridize to a selected miRNA or pre-miRNA target sequence. As used herein, the term “partially double stranded” refers to double stranded structures that contain less nucleotides than the complementary strand. In general, partially double stranded oligonucleotides will have less than 75% double stranded structure, preferably less than 50%, and more preferably less than 25%, 20% or 15% double stranded structure.

An miRNA or pre-miRNA can be 18-100 nucleotides in length, and more preferably from 18-80 nucleotides in length. Mature miRNAs can have a length of 19-30 nucleotides, preferably 21-25 nucleotides, particularly 21, 22, 23, 24, or 25 nucleotides. MicroRNA precursors typically have a length of about 70-100 nucleotides and have a hairpin conformation.

Given the sequence of an miRNA or a pre-miRNA, an miRNA antagonist that is sufficiently complementary to a portion of the miRNA or a pre-miRNA can be designed according to the rules of Watson and Crick base pairing. As used herein, the term “sufficiently complementary” means that two sequences are sufficiently complementary such that a duplex can be formed between them under physiologic conditions. An miRNA antagonist sequence that is sufficiently complementary to an miRNA or pre-miRNA target sequence can be 70%, 80%, 90%, or more identical to the miRNA or pre-miRNA sequence.

In one embodiment, the miRNA antagonist contains no more than 1, 2 or 3 nucleotides that are not complementary to the miRNA or pre-miRNA target sequence. In a preferred embodiment, the miRNA antagonist is 100% complementary to an miRNA or pre-miRNA target sequence.

Useful miRNA antagonists include oligonucleotides have at least 6, 7, 8, 9, 10, 11, 12, 13, 14, 15, 16, 17, 18, 19, 20 or more contiguous nucleotides substantially complementary to an endogenous miRNA or pre-miRNA. The disclosed miRNA antagonists preferably include a nucleotide sequence sufficiently complementary to hybridize to an miRNA target sequence of about 12 to 25 nucleotides, preferably about 15 to 23 nucleotides.

In some embodiments, there will be nucleotide mismatches in the region of complementarity. In a preferred embodiment, the region of complementarity will have no more than 1, 2, 3, 4, or 5 mismatches.

In some embodiments, the miRNA antagonist is “exactly complementary” to a human miRNA. Thus, in one embodiment, the miRNA antagonist can anneal to the miRNA to form a hybrid made exclusively of Watson-Crick base pairs in the region of exact complementarity. Thus, in some embodiments, the miRNA antagonist specifically discriminates a single-nucleotide difference. In this case, the miRNA antagonist only inhibits miRNA activity if exact complementarity is found in the region of the single-nucleotide difference.

In one embodiment, the miRNA antagonists are oligomers or polymers of ribonucleic acid (RNA) or deoxyribonucleic acid (DNA) or modifications thereof. miRNA antagonists include oligonucleotides that contain naturally-occurring nucleobases, sugars and covalent internucleoside (backbone) linkages.

In some embodiments, the miRNA inhibitor is an antagomir. Antagomirs are a specific class of miRNA antagonists that are described, for example, in US2007/0213292 to Stoffel et al. Antagomirs are RNA-like oligonucleotides that contain various modifications for RNase protection and pharmacologic properties such as enhanced tissue and cellular uptake. Antagomirs differ from normal RNA by having complete 2′-O-methylation of sugar, phosphorothioate backbone and a cholesterol-moiety at 3′-end.

Antagomirs can include a phosphorothioate at least the first, second, or third internucleotide linkage at the 5′ or 3′ end of the nucleotide sequence. In one embodiment, antagomirs contain six phosphorothioate backbone modifications; two phosphorothioates are located at the 5′-end and four at the 3′-end. Phosphorothioate modifications provide protection against RNase activity and their lipophilicity contributes to enhanced tissue uptake.

Examples of antagomirs and other miRNA inhibitors are described in WO2009/020771, WO2008/091703, WO2008/046911, WO2008/074328, WO2007/090073, WO2007/027775, WO2007/027894, WO2007/021896, WO2006/093526, WO2006/112872, WO2007/112753, WO2007/112754, WO2005/023986, or WO2005/013901, all of which are hereby incorporated by reference.

Custom designed Anti-miR™ molecules are commercially available from Applied Biosystems. Thus, in some embodiments, the antagomir is an Ambion® Anti-miR™ inhibitor. These molecules are chemically modified and optimized single-stranded nucleic acids designed to specifically inhibit naturally occurring mature miRNA molecules in cells.

Custom designed Dharmacon Meridian™ microRNA Hairpin Inhibitors are also commercially available from Thermo Scientific. These inhibitors include chemical modifications and secondary structure motifs. For example, Vermeulen et al. reports in US2006/0223777 the identification of secondary structural elements that enhance the potency of these molecules. Specifically, incorporation of highly structured, double-stranded flanking regions around the reverse complement core significantly increases inhibitor function and allows for multi-miRNA inhibition at subnanomolar concentrations. Other such improvements in antagomir design are contemplated for use in the disclosed methods.

Anti-HER2 Antibody

In some embodiments, the method further involves administering to the subject a therapeutically effective amount of an anti-HER2 antibody, such as trastuzumab. Trastuzumab, sold under the brand name Herceptin® among others, is a monoclonal antibody used to treat breast cancer that is HER2 receptor positive. It may be used by itself or together with other chemotherapy medication. Trastuzumab is given by slow injection into a vein and injection just under the skin.

Trastuzumab inhibits the effects of overexpression of HER2. If the breast cancer does not overexpress HER2, trastuzumab will have no beneficial effect (and may cause harm). Doctors use laboratory tests to discover whether HER2 is overexpressed. In the routine clinical laboratory, the most commonly employed methods for this are immunohistochemistry (IHC) and either silver, chromogenic or fluorescent in situ hybridisation (SISH/CISH/FISH). HER2 amplification can be detected by virtual karyotyping of formalin-fixed paraffin embedded tumor. Virtual karyotyping has the added advantage of assessing copy number changes throughout the genome, in addition to detecting HER2 amplification (but not overexpression). Numerous PCR-based methodologies have also been described in the literature. It is also possible to estimate HER2 copy number from microarray data.

There are two FDA-approved commercial kits available for HER2 IHC; Dako HercepTest and Ventana Pathway. These are highly standardised, semi-quantitative assays which stratify expression levels into; 0 (<20,000 receptors per cell, no visible expression), 1+(˜100,000 receptors per cell, partial membrane staining, <10% of cells overexpressing HER2), 2+(500,000 receptors per cell, light to moderate complete membrane staining, >10% of cells overexpressing HER2), and 3+(2,000,000 receptors per cell, strong complete membrane staining, >10% of cells overexpressing HER2). The presence of cytoplasmic expression is disregarded. Treatment with trastuzumab is indicated in cases where HER2 expression has a score of 3+. However, IHC has been shown to have numerous limitations, both technical and interpretative, which have been found to impact on the reproducibility and accuracy of results, especially when compared with ISH methodologies. It is also true, however, that some reports have stated that IHC provides excellent correlation between gene copy number and protein expression.

Fluorescent in situ hybridization (FISH) is viewed as being the “gold standard” technique in identifying patients who would benefit from trastuzumab, but it is expensive and requires fluorescence microscopy and an image capture system. The main expense involved with CISH is in the purchase of FDA-approved kits, and as it is not a fluorescent technique it does not require specialist microscopy and slides may be kept permanently. Comparative studies of CISH and FISH have shown that these two techniques show excellent correlation. The lack of a separate chromosome 17 probe on the same section is an issue with regards to acceptance of CISH. The DDISH (Dual-chromagen/Dual-hapten In-situ hybridization) cocktail uses both HER2 and Chromosome 17 hybridization probes for chromagenic visualization on the same tissue section. The detection can be achieved by using a combination of ultraView SISH (silver in-situ hybridization) and ultraView Red ISH for deposition of distinct chromgenic precipitates at the site of DNP or DIG labeled probes.

In some embodiments, HER2 detection a combination of IHC and FISH, whereby IHC scores of 0 and 1+ are negative (no trastuzumab treatment), scores of 3+ are positive (trastuzumab treatment), and score of 2+(equivocal case) is referred to FISH for a definitive treatment decision.

### Oligonucleotides

Disclosed herein are oligonucleotides for use in the disclosed compositions and methods. Compositions and methods for increasing stability of nucleic acid half-life and nuclease resistance are known in the art, and can include one or more modifications or substitutions to the nucleobases, sugars, or linkages of the polynucleotide. For example, the polynucleotide can be custom synthesized to contain properties that are tailored to fit a desired use. Common modifications include, but are not limited to use of locked nucleic acids, unlocked nucleic acids (UNA's), morpholinos, peptide nucleic acids (PNA), phosphorothioate linkages, phosphonoacetate, linkages, propyne analogs, 2′-O-methyl RNA, 5-Me-dC, 2′-5′ linked phosphodiester linage, Chimeric Linkages (Mixed phosphorothioate and phosphodiester linkages and modifications), conjugation with lipid and peptides, and combinations thereof.

In some embodiment, the polynucleotide includes internucleotide linkage modifications such as phosphate analogs having achiral and uncharged intersubunit linkages (e.g., Sterchak, E. P. et al., Organic Chem., 52:4202, (1987)), or uncharged morpholino-based polymers having achiral intersubunit linkages (see, e.g., U.S. Pat. No. 5,034,506). Some internucleotide linkage analogs include morpholidate, acetal, and polyamide-linked heterocycles. Locked nucleic acids (LNA) are modified RNA nucleotides (see, for example, Braasch, et al., Chem. Biol., 8(1):1-7 (2001)). Commercial nucleic acid synthesizers and standard phosphoramidite chemistry are used to make LNAs. Other backbone and linkage modifications include, but are not limited to, phosphorothioates, peptide nucleic acids, tricyclo-DNA, decoy oligonucleotide, ribozymes, spiegelmers (containing L nucleic acids, an apatamer with high binding affinity), or CpG oligomers.

Phosphorothioates (or S-oligos) are a variant of normal DNA in which one of the nonbridging oxygens is replaced by a sulfur. The sulfurization of the internucleotide bond dramatically reduces the action of endo- and exonucleases including 5′ to 3′ and 3′ to 5′ DNA POL 1 exonuclease, nucleases S1 and P1, RNases, serum nucleases and snake venom phosphodiesterase. In addition, the potential for crossing the lipid bilayer increases. Because of these important improvements, phosphorothioates have found increasing application in cell regulation. Phosphorothioates are made by two principal routes: by the action of a solution of elemental sulfur in carbon disulfide on a hydrogen phosphonate, or by the more recent method of sulfurizing phosphite triesters with either tetraethylthiuram disulfide (TETD) or 3H-1,2-bensodithiol-3-one 1,1-dioxide (BDTD). The latter methods avoid the problem of elemental sulfur's insolubility in most organic solvents and the toxicity of carbon disulfide. The TETD and BDTD methods also yield higher purity phosphorothioates. (See generally Uhlmann and Peymann, 1990, Chemical Reviews 90, at pages 545-561 and references cited therein, Padmapriya and Agrawal, 1993, Bioorg. & Med. Chem. Lett. 3, 761).

Peptide nucleic acids (PNA) are molecules in which the phosphate backbone of oligonucleotides is replaced in its entirety by repeating N-(2-aminoethyl)-glycine units and phosphodiester bonds are replaced by peptide bonds. The various heterocyclic bases are linked to the backbone by methylene carbonyl bonds. PNAs maintain spacing of heterocyclic bases that is similar to oligonucleotides, but are achiral and neutrally charged molecules. Peptide nucleic acids are typically comprised of peptide nucleic acid monomers. The heterocyclic bases can be any of the standard bases (uracil, thymine, cytosine, adenine and guanine) or any of the modified heterocyclic bases described below. A PNA can also have one or more peptide or amino acid variations and modifications. Thus, the backbone constituents of PNAs may be peptide linkages, or alternatively, they may be non-peptide linkages. Examples include acetyl caps, amino spacers such as 8-amino-3,6-dioxaoctanoic acid (referred to herein as 0-linkers), and the like. Methods for the chemical assembly of PNAs are well known. See, for example, U.S. Pat. Nos. 5,539,082, 5,527,675, 5,623,049, 5,714,331, 5,736,336, 5,773,571 and 5,786,571.

In some embodiments, the polynucleotide includes one or more chemically-modified heterocyclic bases including, but are not limited to, inosine, 5-(1-propynyl) uracil (pU), 5-(1-propynyl) cytosine (pC), 5-methylcytosine, 8-oxo-adenine, pseudocytosine, pseudoisocytosine, 5- and 2-amino-5-(2′-deoxy-D-ribofuranosyl)pyridine (2-aminopyridine), and various pyrrolo- and pyrazolopyrimidine derivatives, 4-acetylcytosine, 8-hydroxy-N-6-methyladenosine, aziridinylcytosine, 5-(carboxyhydroxylmethyl) uracil, 5-bromouracil, 5-carboxymethylaminomethyl-2-thiouracil, 5-carboxymethylaminomethyluracil, dihydrouracil, N6-isopentenyladenine, 1-methyladenine, 1-methylpseudouracil, 1-methyl guanine, 1-methylinosine, 2,2-dimethylguanine, 2-methyladenine, 2-methylguanine, 3-methylcytosine, N6-methyladenine, 7-methylguanine, 5-methylaminomethyluracil, 5-methoxy-aminomethyl-2-thiouracil, beta-D-mannosylqueosine, 5′-methoxycarbonylmethyluracil, 5-methoxyuracil, 2-methylthio-N6-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, N-uracil-5-oxyacetic acid methylester, 2,6-diaminopurine, and 2′-modified analogs such as, but not limited to O-methyl, amino-, and fluoro-modified analogs. Inhibitory RNAs modified with 2′-fluoro (2′-F) pyrimidines appear to have favorable properties in vitro (Chiu and Rana 2003; Harborth et al. 2003). Moreover, one report recently suggested 2′-F modified siRNAs have enhanced activity in cell culture as compared to 2′-OH containing siRNAs (Chiu and Rana 2003). 2′-F modified siRNAs are functional in mice but that they do not necessarily have enhanced intracellular activity over 2′-OH siRNAs.

In some embodiments the polynucleotide include one or more sugar moiety modifications, including, but are not limited to, 2′-O-aminoethoxy, 2′-O-amonioethyl (2′-OAE), 2′-O-methoxy, 2′-O-methyl, 2-guanidoethyl (2′-OGE), 2′-0,4′-C-methylene (LNA), 2′-O-(methoxyethyl) (2′-OME) and 2′-O—(N-(methyl)acetamido) (2′-OMA).

### Pharmaceutical Compositions

Disclosed is a pharmaceutical compositions containing therapeutically effective amounts of one or more of the disclosed agents and a pharmaceutically acceptable carrier. Pharmaceutical carriers suitable for administration of the agents provided herein include any such carriers known to those skilled in the art to be suitable for the particular mode of administration. In addition, the agents may be formulated as the sole pharmaceutically active ingredient in the agents or may be combined with other active ingredients.

The disclosed agents can be formulated into suitable pharmaceutical preparations such as solutions, suspensions, tablets, dispersible tablets, pills, capsules, powders, sustained release formulations or elixirs, for oral administration or in sterile solutions or suspensions for parenteral administration, as well as transdermal patch preparation and dry powder inhalers. In some embodiments, the agents described above are formulated into pharmaceutical compositions using techniques and procedures well known in the art.

In one embodiment, the compositions are formulated for single dosage administration. To formulate a composition, the weight fraction of compound is dissolved, suspended, dispersed or otherwise mixed in a selected carrier at an effective concentration such that the treated condition is relieved or one or more symptoms are ameliorated.

The active agents is included in the pharmaceutically acceptable carrier in an amount sufficient to exert a therapeutically useful effect in the absence of undesirable side effects on the patient treated. The therapeutically effective concentration may be determined empirically by testing the compounds in in vitro, ex vivo and in vivo systems, and then extrapolated therefrom for dosages for humans.

The concentration of active agents in the pharmaceutical composition will depend on absorption, inactivation and excretion rates of the active compound, the physicochemical characteristics of the agents, the dosage schedule, and amount administered as well as other factors known to those of skill in the art.

Pharmaceutical dosage unit forms are prepared to provide from about 0.01 mg, 0.1 mg or 1 mg to about 500 mg, 1000 mg or 2000 mg, and in one embodiment from about 10 mg to about 500 mg of the active ingredient or a combination of essential ingredients per dosage unit form.

In instances in which the agents exhibit insufficient solubility, methods for solubilizing compounds may be used. Such methods are known to those of skill in this art, and include, but are not limited to, using cosolvents, such as dimethylsulfoxide (DMSO), using surfactants, such as TWEEN®, or dissolution in aqueous sodium bicarbonate.

Liquid pharmaceutically administrable compositions can, for example, be prepared by dissolving, dispersing, or otherwise mixing an active compound as defined above and optional pharmaceutical adjuvants in a carrier, such as, for example, water, saline, aqueous dextrose, glycerol, glycols, ethanol, and the like, to thereby form a solution or suspension. If desired, the pharmaceutical composition to be administered may also contain minor amounts of nontoxic auxiliary substances such as wetting agents, emulsifying agents, solubilizing agents, pH buffering agents and the like, for example, acetate, sodium citrate, cyclodextrin derivatives, sorbitan monolaurate, triethanolamine sodium acetate, triethanolamine oleate, and other such agents.

Dosage forms or compositions containing active ingredient in the range of 0.005% to 100% with the balance made up from non-toxic carrier may be prepared. Methods for preparation of these compositions are known to those skilled in the art. The contemplated compositions may contain 0.001%-100% active ingredient, or in one embodiment 0.1-95%.

### Methods of Administration

The herein disclosed compositions, including pharmaceutical composition, may be administered in a number of ways depending on whether local or systemic treatment is desired, and on the area to be treated. For example, the disclosed compositions can be administered intravenously, intraperitoneally, intramuscularly, subcutaneously, intracavity, or transdermally. The compositions may be administered orally, parenterally (e.g., intravenously), by intramuscular injection, by intraperitoneal injection, transdermally, extracorporeally, ophthalmically, vaginally, rectally, intranasally, topically or the like, including topical intranasal administration or administration by inhalant.

Parenteral administration of the composition, if used, is generally characterized by injection. Injectables can be prepared in conventional forms, either as liquid solutions or suspensions, solid forms suitable for solution of suspension in liquid prior to injection, or as emulsions. A revised approach for parenteral administration involves use of a slow release or sustained release system such that a constant dosage is maintained.

The compositions disclosed herein may be administered prophylactically to patients or subjects who are at risk for a disease or condition. Thus, the method can further comprise identifying a subject at risk for a disease or condition, such as cancer, prior to administration of the herein disclosed compositions.

The exact amount of the compositions required will vary from subject to subject, depending on the species, age, weight and general condition of the subject, the severity of the allergic disorder being treated, the particular nucleic acid or vector used, its mode of administration and the like. Thus, it is not possible to specify an exact amount for every composition. However, an appropriate amount can be determined by one of ordinary skill in the art using only routine experimentation given the teachings herein. For example, effective dosages and schedules for administering the compositions may be determined empirically, and making such determinations is within the skill in the art. The dosage ranges for the administration of the compositions are those large enough to produce the desired effect in which the symptoms disorder are effected. The dosage should not be so large as to cause adverse side effects, such as unwanted cross-reactions, anaphylactic reactions, and the like. Generally, the dosage will vary with the age, condition, sex and extent of the disease in the patient, route of administration, or whether other drugs are included in the regimen, and can be determined by one of skill in the art. The dosage can be adjusted by the individual physician in the event of any counterindications. Dosage can vary, and can be administered in one or more dose administrations daily, for one or several days. Guidance can be found in the literature for appropriate dosages for given classes of pharmaceutical products. In some embodiments, a typical daily dosage of the agent might range from about 1 μg/kg to up to 100 mg/kg of body weight or more per day, depending on the factors mentioned above.

In some embodiments, the disclosed agents are administered in a dose equivalent to parenteral administration of about 0.1 ng to about 100 g per kg of body weight, about 10 ng to about 50 g per kg of body weight, about 100 ng to about 1 g per kg of body weight, from about 1 μg to about 100 mg per kg of body weight, from about 1 μg to about 50 mg per kg of body weight, from about 1 mg to about 500 mg per kg of body weight; and from about 1 mg to about 50 mg per kg of body weight. Alternatively, the amount of agent administered to achieve a therapeutic effective dose is about 0.1 ng, 1 ng, 10 ng, 100 ng, 1 μg, 10 μg, 100 μg, 1 mg, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 30 mg, 40 mg, 50 mg, 60 mg, 70 mg, 80 mg, 90 mg, 100 mg, 500 mg per kg of body weight or greater.

The agent may be administered once or several times a day, and the duration of the treatment may be once per day for a period of about 1, 2, 3, 4, 5, 6, 7 days or more. The agent can also be administered as a single dose in the form of an individual dosage unit or several smaller dosage units or by multiple administration of subdivided dosages at certain intervals. For instance, a dosage unit can be administered from about 0 hours to about 1 hour, about 1 hour to about 24 hours, about 1 to about 72 hours, about 1 to about 120 hours, or about 24 hours to at least about 120 hours. Alternatively, the dosage unit can be administered from about 0.5, 1, 2, 3, 4, 5, 6, 7, 8, 9, 10, 11, 12, 13, 14, 15, 16, 17, 18, 19, 20, 21, 22, 23, 24, 30, 40, 48, 72, 96, 120 hours. Subsequent dosage units can be administered any time following the initial administration such that a therapeutic effect is achieved. Treatment can include a multi-level dosing regimen wherein the agent(s) are administered during two or more time periods, such as having a combined duration of about 12 hours to about 7 days, including, 1, 2, 3, 4, or 5 days or about 15, 15, 30, 35, 40, 45, 50, 55, 60, 65, 70, 75, 80, 85, 90, 95, 100, 105, 110, 115, 120, 125, 130, 135, 140, or 144 hours or about 1 to 24 hours, about 12 to 36 hours, about 24 to 48 hours, about 36 to 60 hours, about 48 to 72 hours, about 60 to 96 hours, about 72 to 108 hours, about 96 to 120 hours, or about 108 to 136 hours.

A number of embodiments of the invention have been described. Nevertheless, it will be understood that various modifications may be made without departing from the spirit and scope of the invention. Accordingly, other embodiments are within the scope of the following claims.

### EXAMPLES

### Example 1: Precise Targeting of POLR2A as a Therapeutic Strategy for Human Triple Negative Breast Cancer

Cancer genomes are characterized by the accumulation of somatic genetic alterations within a cell, such as inactivation of tumour suppressor genes (Chin, L, et al. Gene Dev. 2011 25(6):534-555; Vogelstein, B, et al. 2013 Science 339(6127):1546-1558; Taylor, B S, et al. Cancer Cell 2010 18(1):11-22). TP53 is the most frequently deleted or mutated tumour suppressor gene in TNBC (Shah, S P, et al. Nature 2012 486(7403):395-399; Bianchini, G, et al. Nat. Rev. Clin. Oncol. 2016 13(11):674-690; Weisman, P S, et al. Mod. Pathol. 2016 29:476), which results in the loss of p53's tumour suppressor function (Ventura, A, et al. Nature 2007 445(7128):661-665; Olivier, M, et al. Hum. Mutat. 2002 19(6):607-614). Although restoration of p53 activity is a promising strategy and tremendous efforts have been made to harness it as an anticancer approach, no such therapy has been translated into the clinic owing to the complexity of p53 signalling (Joerger, A C, et al. Annu. Rev. Biochem. 2016 85(1):375-404). Because genomic alterations are large regional events, most cancers that exhibit copy number loss of tumour suppressor genes, especially TP53, also show loss of essential neighbouring genes (Liu, Y, et al. Nature 2015 520(7549):697-701). POLR2A is an essential neighbouring gene of TP53 that encodes the largest subunit of RNA polymerase II complex (Clark, V E, et al. Nat. Genet. 2016 48:1253). Although hemizygous (partial) loss of POLR2A (POLR2A^{loss}) has minimal impact on cells because one allele of POLR2A is sufficient to maintain cell survival, cancer cells containing such genomic defect should be more vulnerable than normal cells to the inhibition of POLR2A. Therefore, this Example precisely targets POLR2A instead of TP53 for treating TNBC harbouring hemizygous loss of TP53 (TP53^{loss}).

In addition, with the aim of enhancing bioavailability and improving endo/lysosomal escape of siRNA, low pH-activated “nano-bomb” nanoparticles were designed to deliver POLR2A siRNA (siPol2) and precisely target POLR2A in TP53^{loss} TNBC. Carbon dioxide (CO₂) can be generated from the nanoparticles under the reduced pH in endo/lysosomes to give the “nano-bomb” effect, which triggers endo/lysosomal escape for enhanced cytosolic siRNA delivery. The disclosed data show that POLR2A suppression with the siPol2-laden nanoparticles (siPol2@NPs) leads to an enhanced reduction of the growth of POLR2A^{loss} tumour with no evident systemic toxicity.

Methods

The Cancer Genome Atlas analysis. The Cancer Genome Atlas primary (origin: METABRIC Nature 2012 & Nat Commun 2016, and Cell 2015) and metastatic (origin: France 2016) breast cancer data were downloaded from cBioPortal, which included copy number variation at segment levels in log-ratio, copy number variation at gene levels estimated by using the GISTIC2 algorithm, RNA-seq for gene expression in base-2 log scale, and patient information on oestrogen receptor, progesterone receptor, and HER-2/neu status. The correlation between gene copy number and the corresponding gene expression was analysed as previously described (Liu, Y., et al. Nature 2015 520(7549):697-701). The triple-negative breast cancer (TNBC) subtypes, defined by PAM50 profiling test, included all the basal-like and claudin-low groups.

Materials. PLGA (lactide:glycolide: 75:25, M_{w}: 4,000-15,000 Da), and organic solvents were purchased from Sigma (St. Louis, Mo., USA). Agarose, ethidium bromide and loading buffer were purchased from Thermo Fisher Scientific (Grand Island, N.Y., USA). DPPC was purchased from Anatrace (Maumee, Ohio, USA). Chitosan oligosaccharide of pharmaceutical grade (M_{w}: 1.2 kDa, 95% deacetylation) was purchased from Zhejiang Golden Schell Biochemical Co. Ltd (Zhejiang, China). Methyl aminomethanimidothioate hydroiodide was purchased from Santa Cruz Biotechnology (Dallas, Tex., USA). Anti-POLR2A antibody (sc-47701, dilution of 1:10000 for western blot and 1:200 for immunofluorescence staining), HRP-anti-rabbit IgG (sc-2030, dilution of 1:5000), HRP-anti-mouse IgG (sc-516102, dilution of 1:5000) antibodies were purchased from Santa Cruz (Dallas, Tex., USA). Anti-β-actin (AM1829B, dilution of 1:5000) antibody was purchased from Abgent (San Diego, Calif., USA). Alexa Fluor 488-labeled anti-mouse IgG (A11001, dilution of 1:250) antibody was purchased from Life Technologies (Waltham, Mass., USA). Cell counting kit-8 (CCK-8) was purchased from Dojindo Molecular Technologies (Rockville, Md., USA). Mouse Inflammation Kit (#552364) was purchased from BD Biosciences (San Jose, Calif., USA). Alanine Transaminase (ALT) Activity Assay Kit (ab105134) and Aspartate Aminotransferase (AST) Activity Assay Kit (ab105135) were purchased from Abcam (Cambridge, Mass., USA).

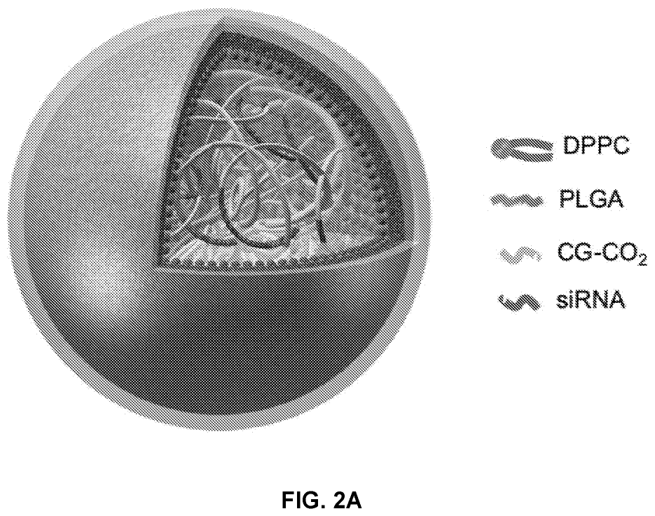

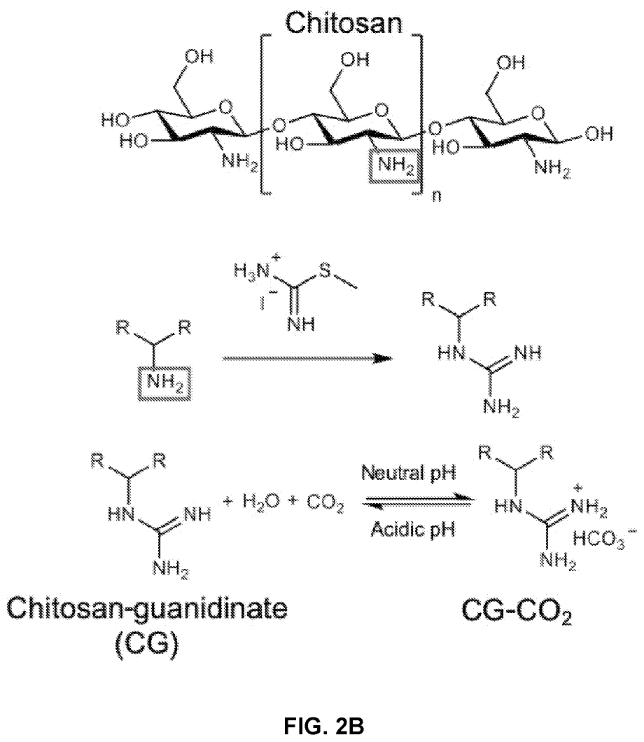

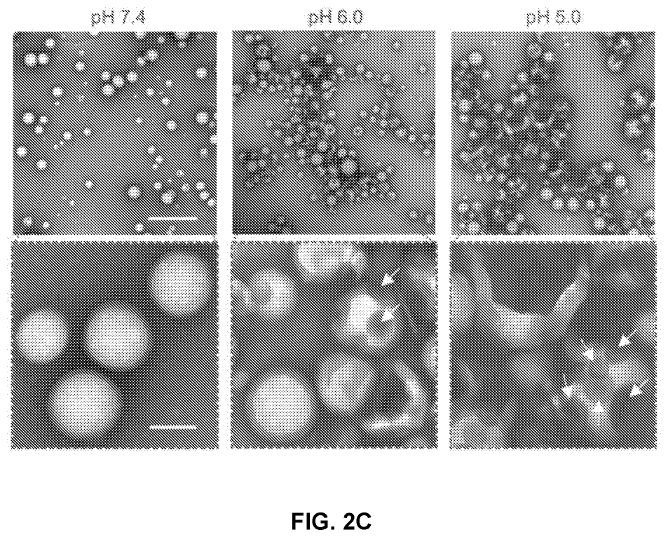

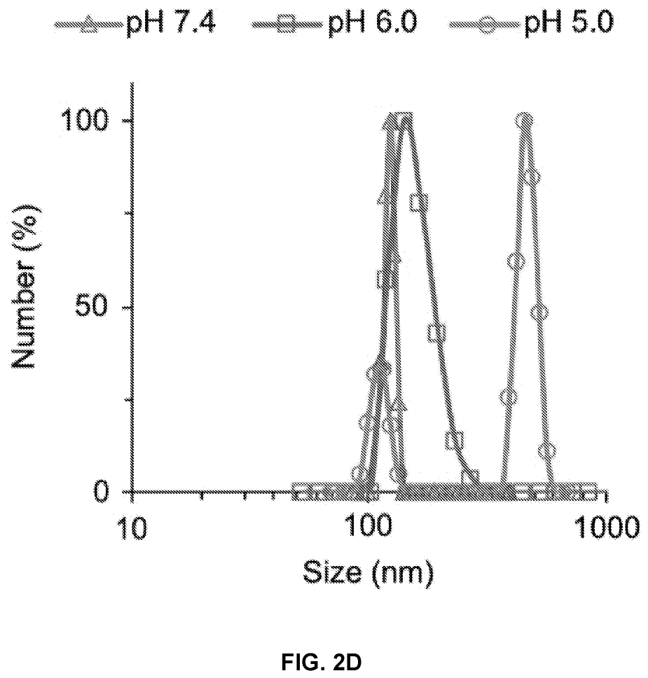

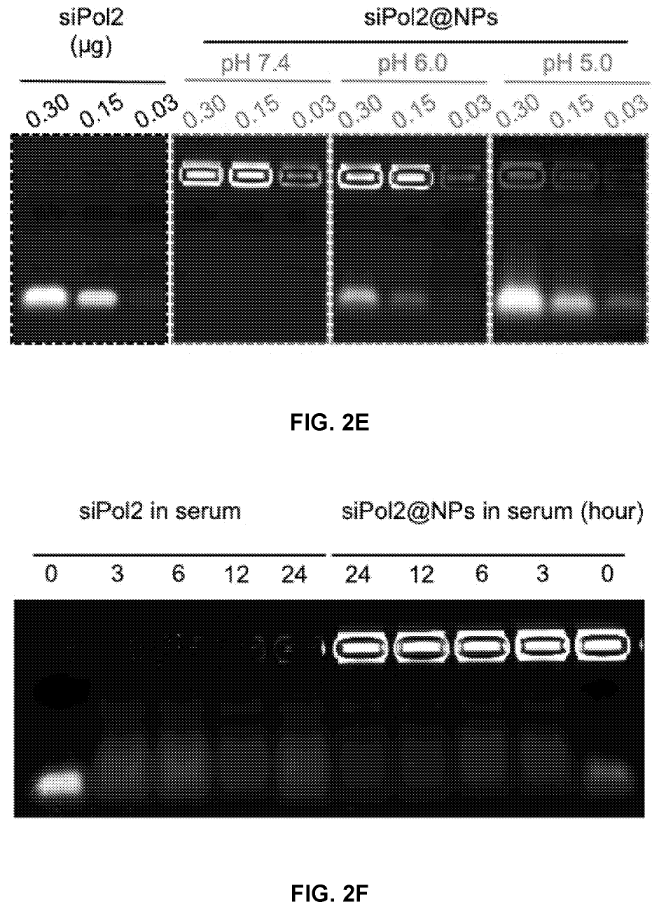

Nanoparticle synthesis. The double-emulsion method (Wang, H, et al. Chem. Commun. 2015 51(36):7733-7736) with slight modification was used to synthesize nanoparticles in this example. First, the chitosan-guanidinate (CG) was synthesized by modifying chitosan with the guanidine group (FIG. 2B). The guanidine group is a common functional group in many natural products including the naturally occurring amino acid L-arginine. Briefly, 480 mg of chitosan (1.2 kDa) was dissolved in 12 ml of deionised (DI) water. A total of 6 ml of methyl aminomethanimidothioate hydroiodide in acetonitrile (145 mg ml⁻¹) was then added and stirred at room temperature for 24 h under N₂ atmosphere. Afterward, the solvent and side-product were removed under vacuum. Lastly, to remove the unreacted methyl aminomethanimidothioate hydroiodide, the solid was dissolved in 1 ml of DI water and dropped into 10 ml of tetrahydrofuran (THF), filtered, and the final solid CG was collected for further use. For capturing CO₂, CG-CO₂ was made by bubbling the aqueous solution of CG for 1 h with CO₂. To synthesize nanoparticles, 10 mg of DPPC and 20 mg of PLGA were dissolved in dichloromethane (DCM, 2 ml). This organic solution and 400 μl of DI water dissolved with 1 mg ml⁻¹ CG-CO₂ and 500 μg ml⁻¹ POLR2A siRNA (without or with Cy5.5 modification, Sigma) were transferred into a centrifuge tube (50 ml). The sample was then emulsified for 1 min by sonication with a Branson 450 sonifier at 18% power. A total of 8 ml of PVA solution (2% in DI water) was then added into the centrifuge tube and the sample was further emulsified as aforementioned for 1 min. Afterward, the organic solvent (i.e, DCM) was removed by rotary evaporation. The nanoparticles were then collected by centrifugation for 10 min at 10000 g and washed two times in DI water at room temperature.

Nanoparticle characterization. Both dynamic light scattering (DLS) and transmission electron microscopy (TEM) were used to characterize the nanoparticles. The nanoparticles were soaked for 6 h in either acetate buffer (pH 5.0) or phosphate buffer (for pH 6.0 and pH 7.4). For TEM study, uranyl acetate solution (2%, w/w) was used to negatively stain the nanoparticles before examining them with the Tecnai G2 Spirit transmission electron microscope from FEI (Moorestown, N.J., USA). The size distribution of the nanoparticles was studied using a 90 Plus/BI-MAS DLS instrument from Brookhaven (Holtsville, N.Y., USA). The encapsulation efficiency of the siRNA was 68.6±7.2%, which was calculated as the ratio of the amount of the siRNA encapsulated in the nanoparticles to that the total amount of the siRNA fed for encapsulation. The loading content of the siRNA in the nanoparticles was 0.73±0.25%, which calculated as the ratio of the amount of the siRNA encapsulated in the nanoparticles to that the total amount of the nanoparticles including the siRNA. Both the encapsulation efficiency and loading content were quantified by using Cy5.5-siPol2 for encapsulation. The amount of Cy5.5-siPol2 in a sample was measured spectrophotometrically using a Beckman Coulter (Indianapolis, Ind., USA) DU 800 UV-vis Spectrophotometer based on its absorbance peak at 670 nm. A standard curve of free Cy5.5-siPol2 (absorbance vs. concentration) was used for converting the measured absorbance into the concentration of Cy5.5-siPol2 in a sample.

Electrophoretic gel assay. Free POLR2A siRNA (siPol2) and siPol2@NPs (in phosphate buffered saline or serum) were mixed with loading buffer, and then loaded into 2% wt agarose gel with 0.5 mg ml⁻¹ ethidium bromide. Electrophoresis was conducted in 1×tris-acetate-EDTA (TAE) buffer at 80 V for 10 min. The resulting gels were analysed using a UV illuminator (FluorChem™ E System, CA, USA) to show the location of siPol2.

Cell culture. MDA-MB-231, MDA-MB-453, and HCC1937 cell lines were purchased from the American Type Culture Collection (ATCC, Manassas, Va., USA) and cultured under the standard conditions specified by ATCC. HER18 cells (stably overexpress HER2) were a gift from Dr. Mong-Hong Lee (MD Anderson Cancer Centre). The cells were maintained in Dulbecco's Modified Eagle Medium (DMEM) with 10% fetal bovine serum at 37° C. in 5% CO₂. Cell identity was confirmed by validating the STR DNA fingerprinting using the AmpFLSTR Identifiler Kit (Applied Biosystems, Foster City, Calif., USA) according to the manufacturer's instructions.

Generation of POLR2A-heterozygous cell lines. The generation of POLR2A^{loss} isogenic cell lines was conducted using CRISPR/Cas9 as described previously (Liu, Y., et al. Nature 2015 520(7549):697-701). Briefly, 2×10⁶ of HCC1937 or HER18 cells were transfected with 2 μg of Cas9/sgRNA-expressing vector DNA using a Nucleofector kit (Lonza, Walkersville, Md., USA). Genome editing efficacy was tested by the Surveyor assay. Single colonies were isolated and the PCR products from positive clones were ligated to the pGEM-T Easy Vector (Promega, Madison, Wis., USA) and further confirmed by Sanger DNA sequencing.

Cellular uptake and intracellular distribution. The nanoparticles were encapsulated with Cy5.5-siPol2 using the aforementioned double-emulsion method. For the nanoparticles without CO₂, CG was used instead of CG-CO₂. MDA-MB-453 cells were then treated with the Cy5.5-siPol2@NPs at 37° C. for up to 6 h. Afterward, the cells were incubated at 37° C. for 30 min with 50 nM DAPI and 90 nM LysoTracker Green in cell culture medium. The cells were then fixed with 4% paraformaldehyde and examined using a FluoView FV1000 confocal microscope from Olympus (Centre Valley, Pa., USA). For quantitative analysis of the co-localization between Cy5.5-siPol2 and LysoTracker Green inside cells, the Manders' co-localization coefficients between the fluorescence signals of Cy5.5-siPol2 and LysoTracker Green were calculated using ImageJ (ImageJ bundled with 64-bit Jave 1.8.0_112) with the JACoP co-localization plugin module. The two Manders' coefficients M1 and M2 were calculated as follows:

$$\begin{matrix}M 1 = \frac{\sum {R}_{i , co - local}}{\sum {R}_{i}} & ( 1 ) \\ {R}_{i , co - local} = {R}_{i} if {R}_{i} > {T}_{1} and {G}_{i} > {T}_{2} & ( 2 ) \\ {R}_{i} = Red fluorescence intensity if {R}_{i} > {T}_{1} & ( 3 ) \\ M 2 = \frac{\sum {G}_{i , co - l o c a l}}{\sum {G}_{i}} & ( 4 ) \\ {G}_{i , co - local} = {G}_{i} if {G}_{i} > {T}_{2} and {R}_{i} > {T}_{1} & ( 5 ) \\ {G}_{i} = Green fluorescence intensity if {G}_{i} > {T}_{2} & ( 6 )\end{matrix}$$

where the subscript i represents the ith pixel in the fluorescence image, R represents red fluorescence intensity, G represents green fluorescence intensity, T₁ represents the threshold for red channel, and T₂ represents the threshold for green channel. The two fluorescence intensities and thresholds were determined by the built-in algorithm of the JACoP co-localization plugin module of ImageJ for both the green and red channels.

Immunoblotting. Immunoblotting was performed as described previously (Liu, Y., et al. Nature 2015 520(7549):697-701). Briefly, cell lysates were made in lysis buffer (pH 7.5) containing 1 mM EDTA, 50 mM Tris, 150 mM NaCl, 0.5% Triton X-100, 0.5% NP-40, 1 mM sodium fluoride, 1 mM phenylmethylsulfonyl fluoride, 5 mM sodium vanadate, and 1 μg/ml leupeptin, aprotinin, and pepstatin. Proteins were then separated by SDS-PAGE gels and further transferred to the membranes of polyvinylidene difluoride (Bio-Rad, Hercules, Calif., USA). After blocking the membranes with 5% nonfat milk for 1 h at room temperature, they were incubated with primary antibodies as indicated. The membranes were then washed and incubated at room temperature with peroxidase-conjugated secondary antibodies for 1 h. After 5 times of washing, immunodetected bands on the membranes were visualized by taking chemiluminescent images on X-ray films with the enhanced chemiluminescence (ECL) system (PerkinElmer, Waltham, Mass., USA) as per the manufacturer's instructions.

Cell viability and cell colony formation assays. Equal numbers of cells were plated in 96 well plates in triplicate. After incubation with indicated treatments for 72 hours, cell viability was quantified using the CCK-8 according to the manufacturer's instructions. For the cell colony formation assay, cell colonies were visualized using crystal violet (Fisher scientific, Pittsburgh, Pa., USA) according to the manufacturer's instructions.

Animals. Female NU/NU nude mice (6 weeks old) were purchased from either Charles River (Wilmington, Mass., USA) for the TNBC tumour models or The Jackson Laboratory (Bar Harbour, Me., USA) for the HER2+ tumour model. Wild type C57BL/6J mice (6 weeks old) were purchased from The Jackson Laboratory. All animals were maintained 16:8 h of light-dark cycle. All animal studies were conducted by following protocols approved by the Institutional Animal Care and Use Committee (IACUC) at The Ohio State University and Indiana University School of Medicine. The animal protocols are compliant with all relevant ethical regulations.

Orthotopic xenograft breast tumour models and treatments. For wild type POLR2A^{loss}/POLR2A^{neutral} orthotopic xenograft TNBC model, nude mice were injected with 1×10⁶ MDA-MB-453 cancer cells into the 4^{th} inguinal mammary fat pad on the left and 1×10⁶ MDA-MB-231 cancer cells into the 4^{th} inguinal mammary fat pad on the right on the same day. For isogenic POLR2A^{loss}/POLR2A^{neutral} orthotopic xenograft TNBC model, nude mice were injected with 1×10⁶ HCC1937 (POLR2A^{loss}) cancer cells into the 4^{th} inguinal mammary fat pad on the left and HCC1937 (POLR2A^{neutral}) cancer cells into the 4^{th} inguinal mammary fat pad on the right on the same day. For isogenic POLR2A^{loss}/POLR2A^{neutral} orthotopic xenograft HER2+ breast cancer model, nude mice were injected with 5×10⁶ HER18 (POLR2A^{loss}) cancer cells into the 4^{th} inguinal mammary fat pad on the left and HER18 (POLR2A^{neutral}) cancer cells into the 4^{th} inguinal mammary fat pad on the right. The nude mice were supplemented with weekly subcutaneous oestradiol cypionate injections (3 mg kg⁻¹ per week), starting 1 week prior to injection of tumour cells. After initial tumour establishment (˜100 mm³), mice were randomly grouped and treated with indicated formulations. Treatment formulations were administered twice weekly by intravenous injection via the tail vein, and tumour size and body weight were monitored biweekly. The dose of siPol2 for any formulations with the siRNA was 0.3 mg kg⁻¹ body weight. Tumour size was measured using a calliper, and tumour volume was calculated using the standard formula: 0.5×L×W², where L is the long diameter and W is the short diameter. Mice were euthanized when they met the institutional euthanasia criteria for tumour size and overall health condition. Tumours were removed, photographed, and weighed. The freshly dissected tumour tissues were either used for western blot analysis or fixed in 10% buffered formalin overnight, transferred to 70% ethanol, embedded in paraffin, sectioned and stained with hematoxylin and eosin (H&E) and indicated antibodies.

Biodistribution of nanoparticles in vivo. After initial tumour establishment (˜100 mm³), mice were injected with either 100 μl of saline, Cy5.5-siPol2 (50 μg) in 100 μl of saline, or Cy5.5-siPol2@NPs (nanoparticles containing 50 μg of Cy5.5-siPol2) in 100 μl of saline. Images were taken immediately before injection and at 2 and 8 h after intravenous injection via the tail vein using an in vivo imaging system (Perkin Elmer IVIS, Waltham, Mass., USA) with an excitation at 675 nm and a 690-770 nm Cy5.5-filter to collect the fluorescence emission of Cy5.5. For ex vivo imaging, the mice were sacrificed after in vivo imaging at 8 h, and tumor and main organs were harvested for further fluorescence imaging using the same in vivo imaging system.

Detection of immune response induction by nanoparticles. Wild type C57BL/6J mice (6 weeks old) purchased from The Jackson Laboratory were randomized and injected intravenously with the following treatments: 1) saline, 2) free siPol2, 3) siNT@NPs, and 4) siPol2@NPs. At indicated time points, blood was drawn from mice and the concentration of indicated cytokines in serum was measured by BD Mouse Inflammation Kit (#552364), ALT Activity Assay Kit (Abcam, ab105134), and AST Activity Assay Kit (Abcam, ab105135) according to the manufacturer's instructions.

Statistics and reproducibility. Each experiment was repeated independently for at least three times. Unless otherwise noted, data are presented as mean±standard deviation (s.d.) or standard error of mean (s.e.m.). Student's t-test (unpaired and two-tailed) was used to compare two groups of independent samples, assuming equal variance with no samples being excluded from the analysis. One-way analysis of variance (ANOVA) with Dunnett's post hoc analysis was used for multiple comparison (when more than two groups compared). One-way ANOVA with Fisher's LSD post hoc was used for tumour growth analysis. Statistical methods used for The Cancer Genome Atlas data analysis were described above. All statistical analyses were carried out with Prism (version 7.0, GraphPad Software, San Diego, Calif., USA). A p value less than 0.05 was considered statistically significant.

Results

Hemizygous Deletion of POLR2A and TP53 in Breast Cancer

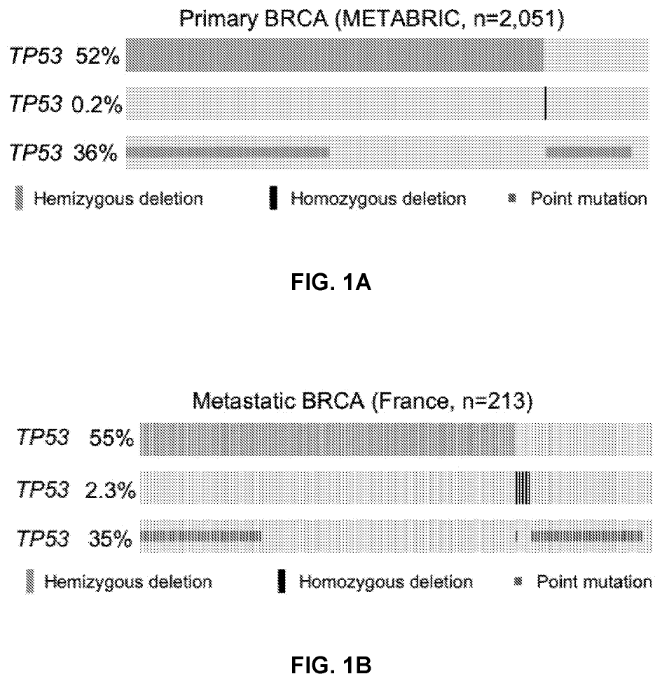

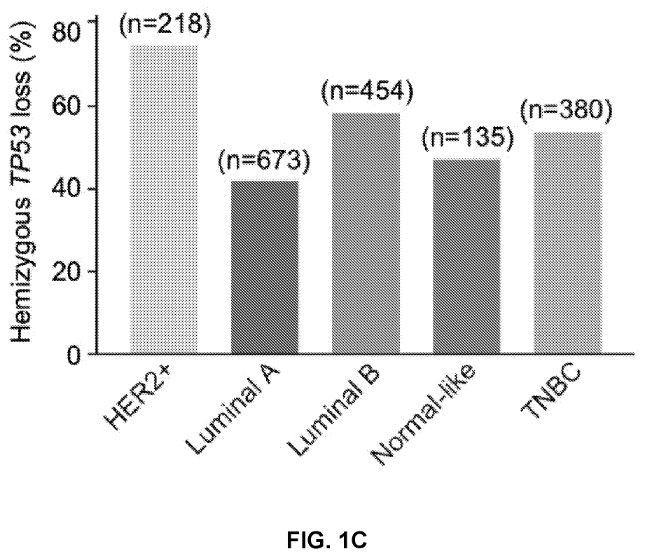

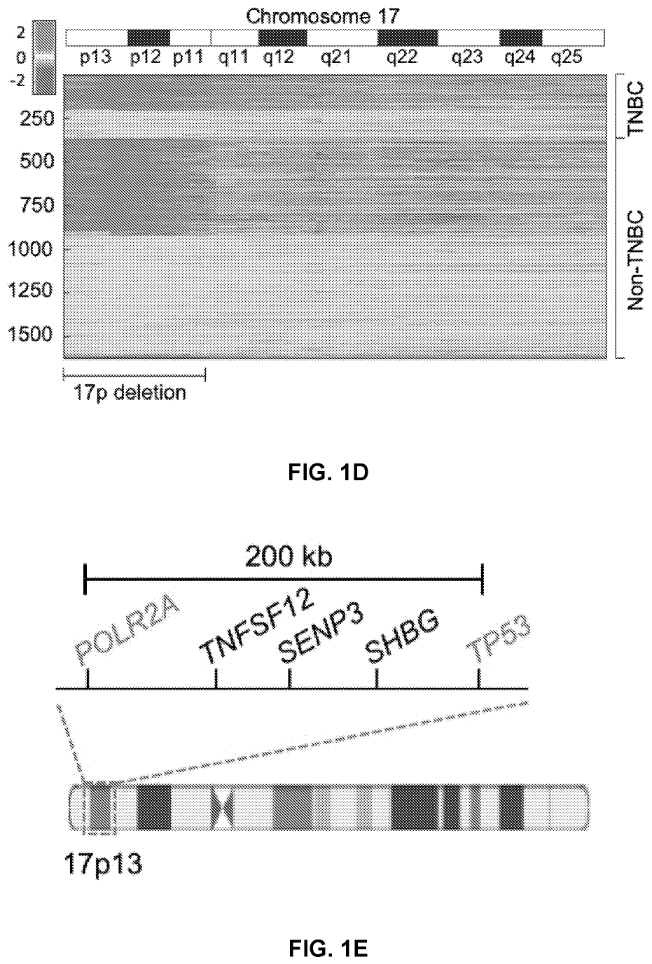

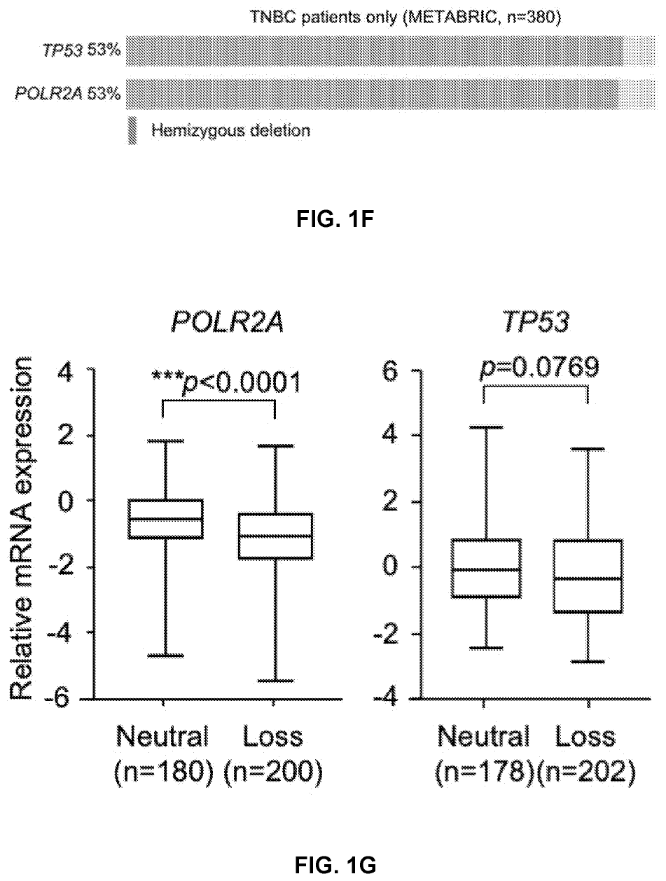

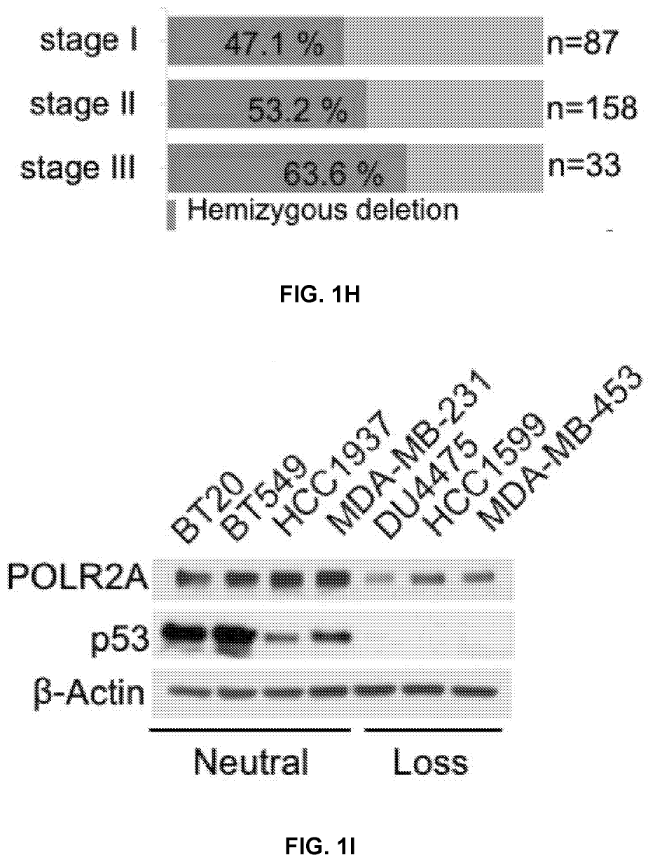

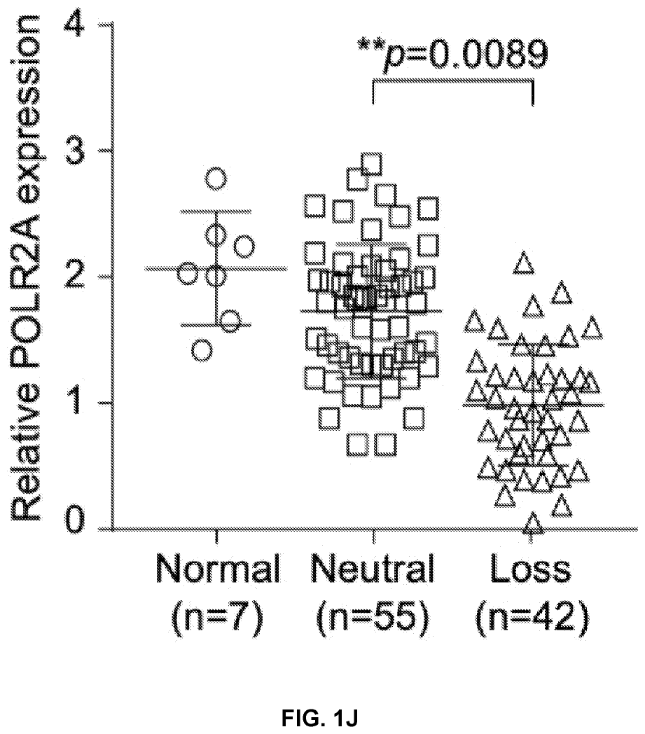

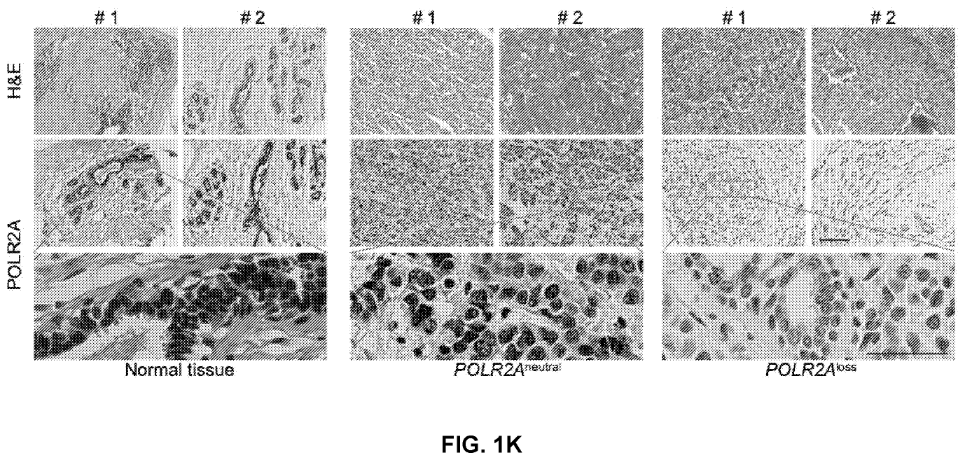

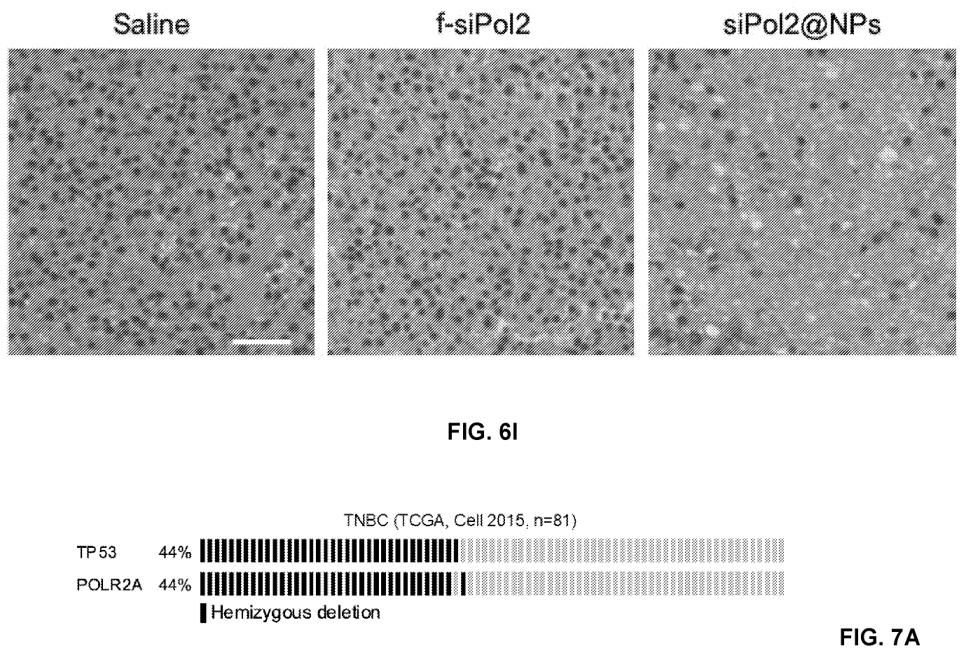

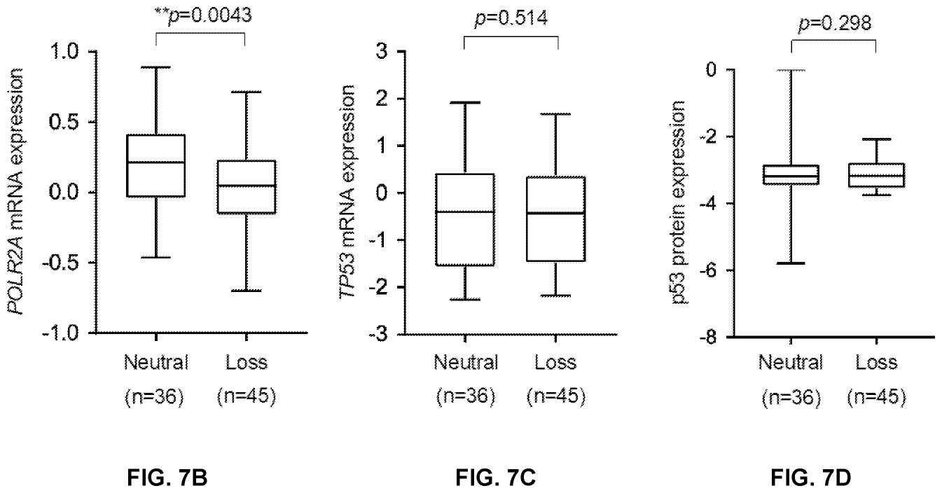

Inactivation of TP53 is a frequent event in most human tumours (Liu, Y, et al. Nature 2015 520(7549):697-701). However, neither TP53 mutation nor complete deletion of TP53 is the most frequent in primary human breast cancers. There are only 36% (741 out of 2,051) and 0.5% (5 out of 2,051) cases for mutation and homozygous deletion, respectively (FIG. 1A). On the contrary, hemizygous deletion of TP53 is highly frequent in both primary and metastatic breast cancers (52% and 55%, respectively, FIGS. 1A, 1B). Particularly, 53% (202 out 380) of TNBC cases carry the hemizygous deletion of TP53 (FIG. 1C). Analyses of the breast cancer genomes show that the TP53 deletion is within a large fragment deletion of Chr17p spanning over ˜19.8 megabases of DNA in TNBC and other human breast cancers (FIGS. 1D, 1E). The POLR2A and TP53 genes are nearly always co-deleted in the Chr17p deletion region (FIG. 1F). Importantly, the POLR2A expression levels are significantly correlated with the copy numbers of POLR2A in the TNBC subtype while this correlation is not significant for p53 (FIGS. 1G, 7). Although one allele deletion of the Chr17p fragment significantly decreases the POLR2A mRNA expression, more severe stages of TNBC are associated with increased frequencies of patients with hemizygous TP53 loss (FIG. 1H). This indicates one copy of POLR2A is sufficient to maintain cell survival. This positive correlation is validated in several TNBC cell lines (FIG. 1I). In immunohistochemical analysis using a tumour tissue microarray containing 100 TNBC samples, the expression of POLR2A was scored according to the staining intensity and proportion of signals in each sample. Accordingly, the copy numbers of POLR2A in the tumour tissue samples were determined by quantitative polymerase chain reaction (PCR). A tight correlation was validated between POLR2A copy numbers and protein expression levels (FIG. 1J, 1K). Collectively, these data suggest inhibition of POLR2A is an amenable approach for targeted treatment of TNBC.

Synthesis and Characterization of Nanoparticles

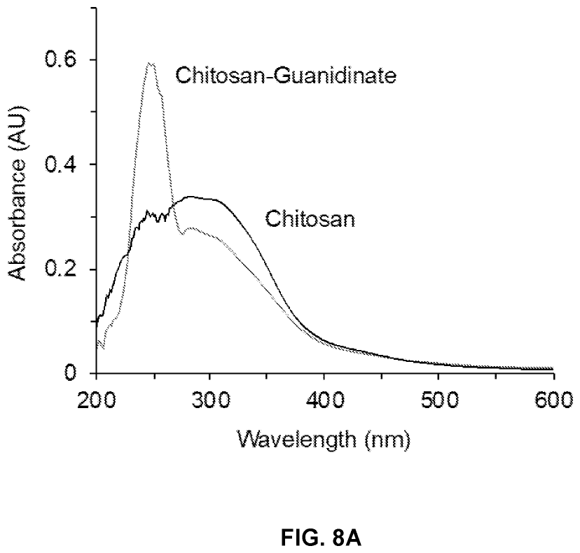

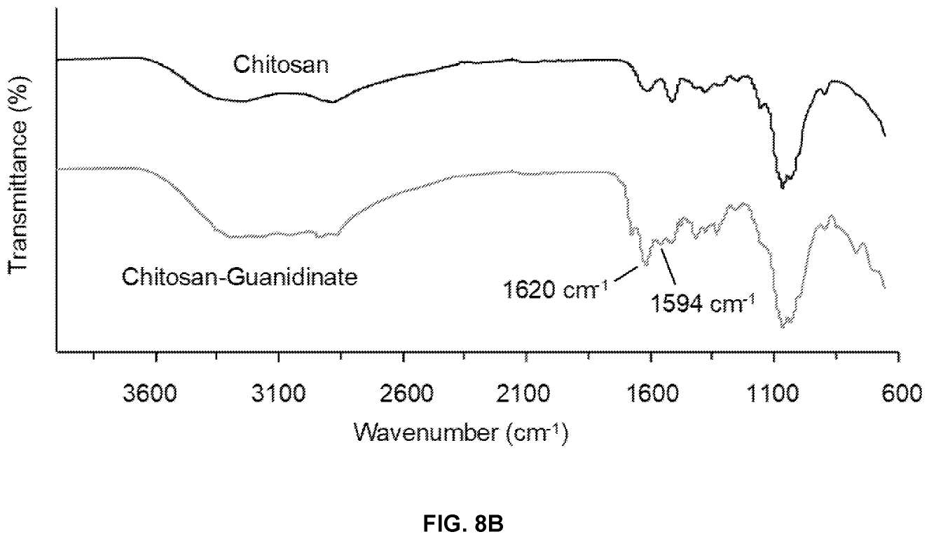

A core-shell nanoplatform illustrated in FIG. 2A was developed for delivering siPol2 to target POLR2A. The core contains siRNA and chitosan modified with guanidine (chitosan-guanidinate or CG, FIGS. 2B, 8). The guanidine group can react reversibly with CO₂ to form chitosan-guanidinate-CO₂ (CG-CO₂) in a pH dependent manner (Seipp, C A, et al. Angew. Chem. Int. Ed. Engl. 2017 56(4):1042-1045), which is utilized to capture/store CO₂ at neutral pH for release at reduced pH such as that (˜5) inside endo/lysosomes (FIG. 2B).

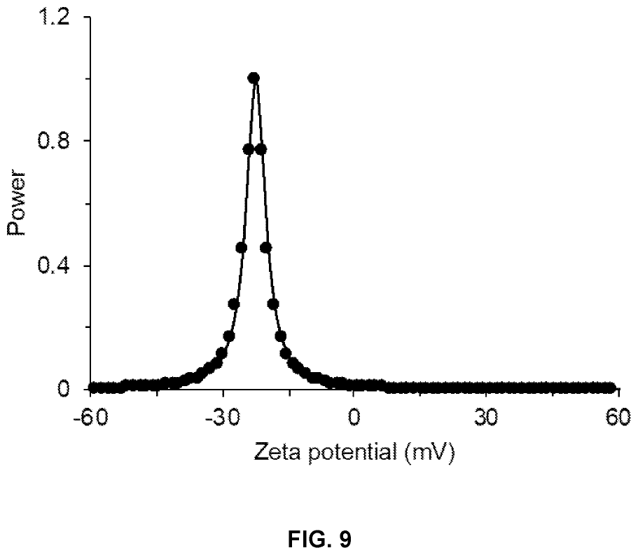

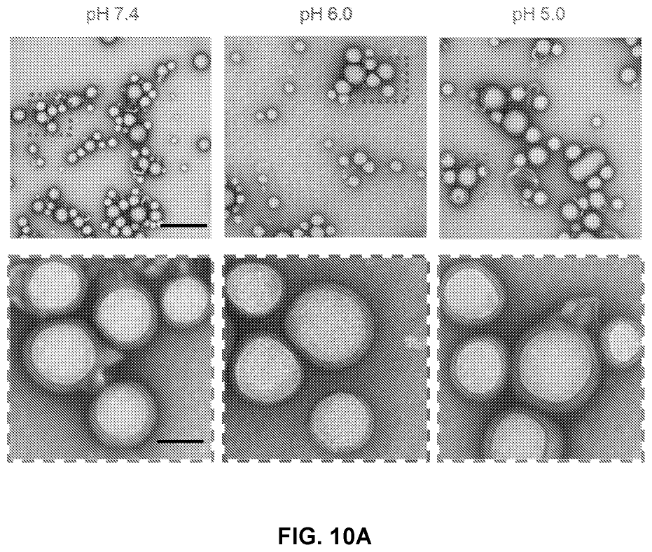

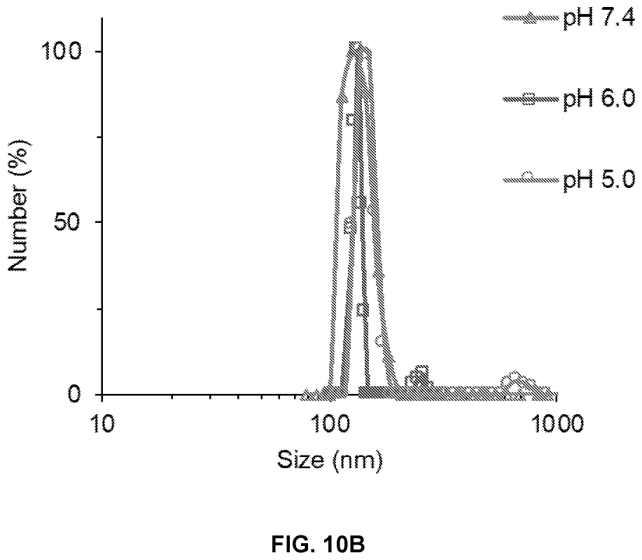

Typical transmission electron microscopy images of the siPol2-laden nanoparticles (siPol2@NPs) are shown in FIG. 2C. The siPol2@NPs have a spherical morphology and core-shell structure. The nanoparticles are stable at neutral pH and 110±5.7 nm in diameter with a narrow size distribution (FIGS. 2C, 2D) and negatively charged surface (or surface zeta potential: −22.4±2.1 mV, FIG. 9). The size of the nanoparticles synthesized using CG without CO₂ is not significantly affected by either pH 6.0 or pH 5.0 (FIG. 10). In contrast, when CG-CO₂ is used, their size changes to 138±25.2 nm and defects (white arrows, FIG. 2C) show up on some of the nanoparticles at pH 6.0. When the pH is further reduced to 5.0, the size increases by >4 times to 463±54.8 nm for most nanoparticles (resulting in an additional peak, FIG. 2D) and defects show up on all nanoparticles (FIG. 2C). These results indicate that the CO₂ generation from CG-CO₂ encapsulated in the nanoparticles under low pH (particularly pH 5.0) conditions could expand and/or break open the nanoparticles, to give the “nano-bomb” effect. It is also possible that the damaged nanoparticles might form aggregates at pH 5.0, which might contribute to the additional peak at ˜450 nm shown in FIG. 2D.

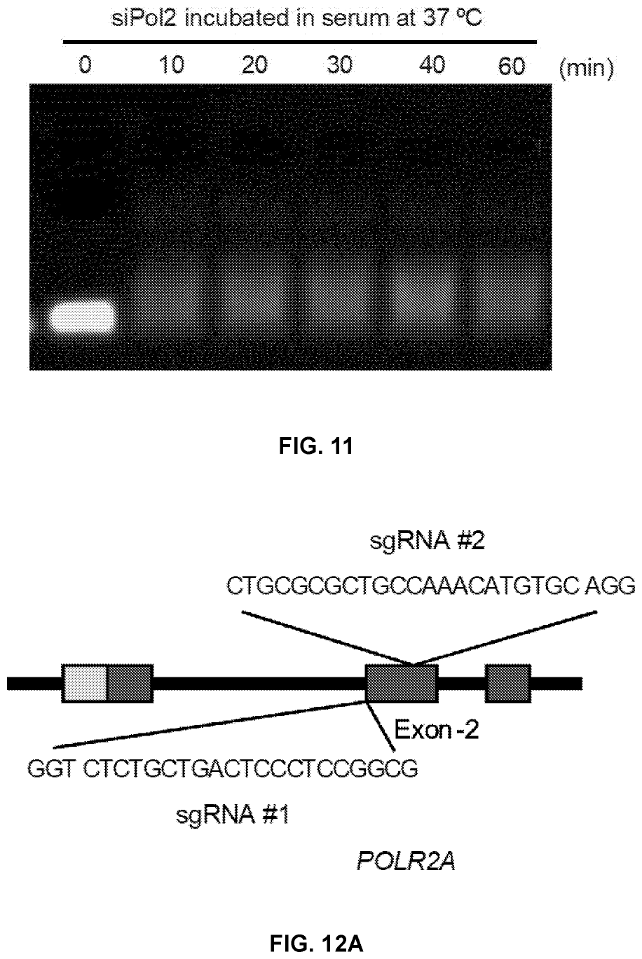

The migration of siPol2 into electrophoretic agarose gel is almost completely inhibited by nanoparticle encapsulation with negligible release at pH 7.4 (FIG. 2E). Moreover, the strong signal observed under pH 5.0 closely resembles the free siPol2 band. Under pH 6.0, smaller bands indicating slower release of the siRNA could be seen. These data demonstrate the low pH activated “nano-bomb” effect of the nanoparticles could trigger the release of the encapsulated siPol2. Furthermore, the siPol2@NPs were observable for at least 24 h in serum while most of the free siPol2 degraded within 10 min and no siPol2 was discernible after 3 h (FIGS. 2F, 11). These data indicate the nanoparticles could enhance the stability of the siPol2 in blood and allow for pH triggered release of the siRNA.

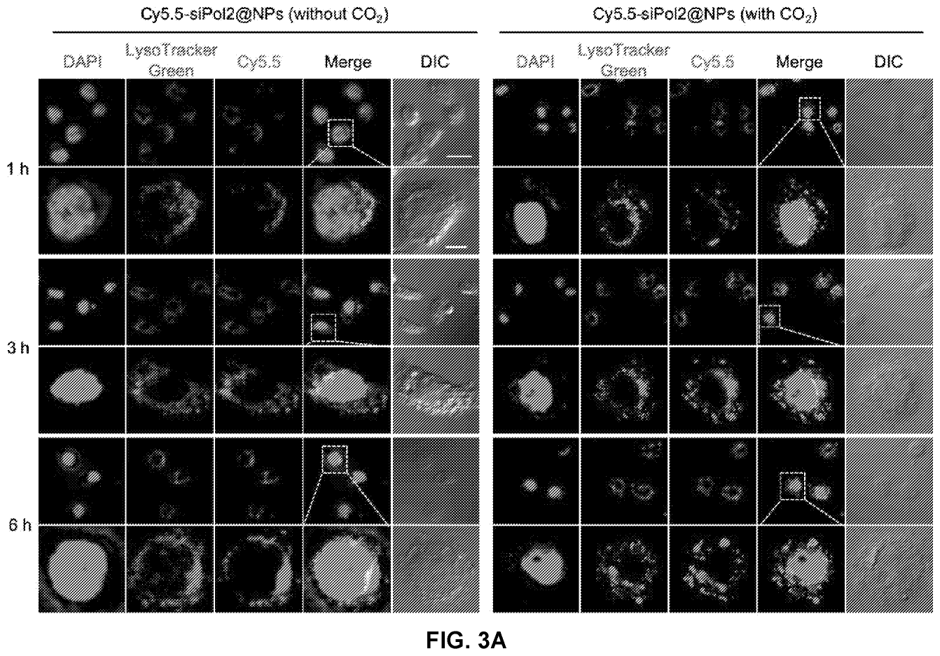

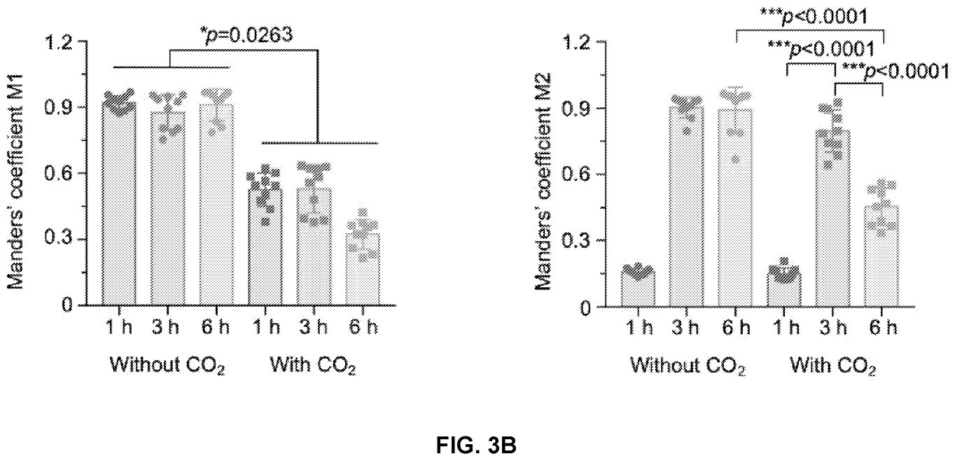

To examine cellular uptake and intracellular trafficking, siPol2 was labelled with a red fluorescence probe (Cy5.5) and encapsulated in the nanoparticles. In MDA-MB-453 TNBC cells treated using nanoparticles without CO₂, the red fluorescence of Cy5.5 overlaps with the green fluorescence of LysoTracker Green that stains the endo/lysosomes at all time points (FIG. 3A, left panel). In stark contrast, the overlap between the green and red fluorescence is reduced in the cells treated using nanoparticles with CO₂ at all the three time points (particularly at 6 h) (FIG. 3A, right panel), indicating successful escape of the Cy5.5-siPol2 from endo/lysosomes. These observations are confirmed by quantitative analyses of co-localization of Cy5.5-siPol2 with endo/lysosomes in the confocal fluorescence images using the Manders' coefficients M1 and M2 (Manders, E M M, et al. J. Microsc. 1993 169(3):375-382). As shown in FIG. 3B, M1 is close to 1 at all the three time points for the condition without CO₂, indicating the siRNA encapsulated in the nanoparticles without CO₂ was taken up via endocytosis and stayed in endo/lysosomes. In contrast, the M1 is much less than 1 at all the three time points (particularly 6 h) for the condition with CO₂, indicating the CO₂ generation in the nanoparticles can result in effective endo/lysosomal escape of the encapsulated siRNA. The M2 data show nearly all of the endo/lysosomes contained Cy5.5-siPol2 at 3 and 6 h in the absence of CO₂ generation. For the condition with CO₂ generation, the M2 increases first and then decreases. This is because the uptake of Cy5.5-siPol2 was faster than its endo/lysosomal escape in the first 3 h, and after that, the rate of endo/lysosomal escape of Cy5.5-siPol2 surpasses its uptake. Collectively, these data suggest the low pH triggered nano-bomb effect of the nanoparticles facilitates endo/lysosomal escape for cytosolic delivery of siRNAs.

Suppression of POLR2A Inhibits POLR2A^{loss} Cancer Cell Growth

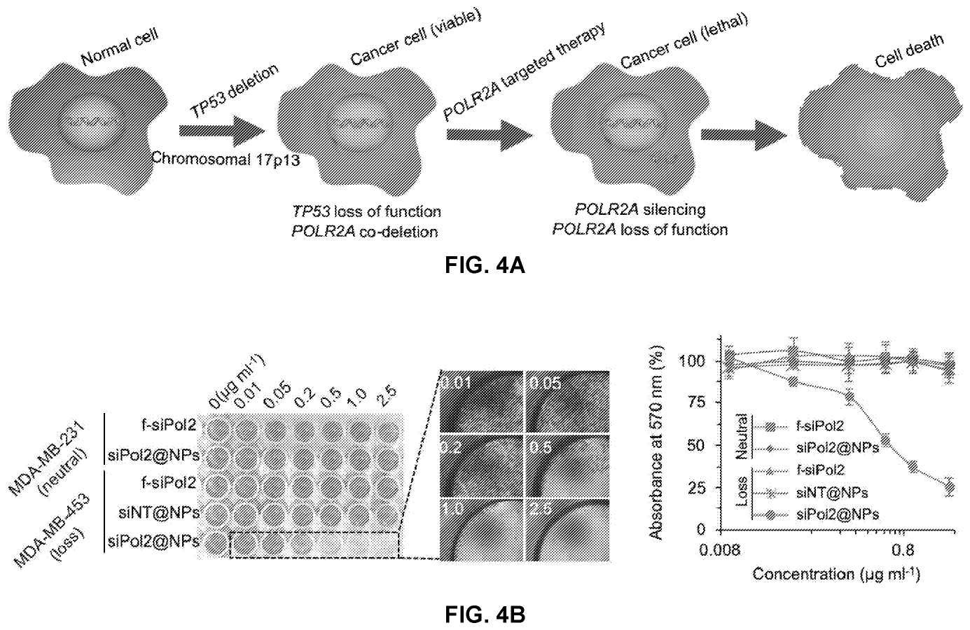

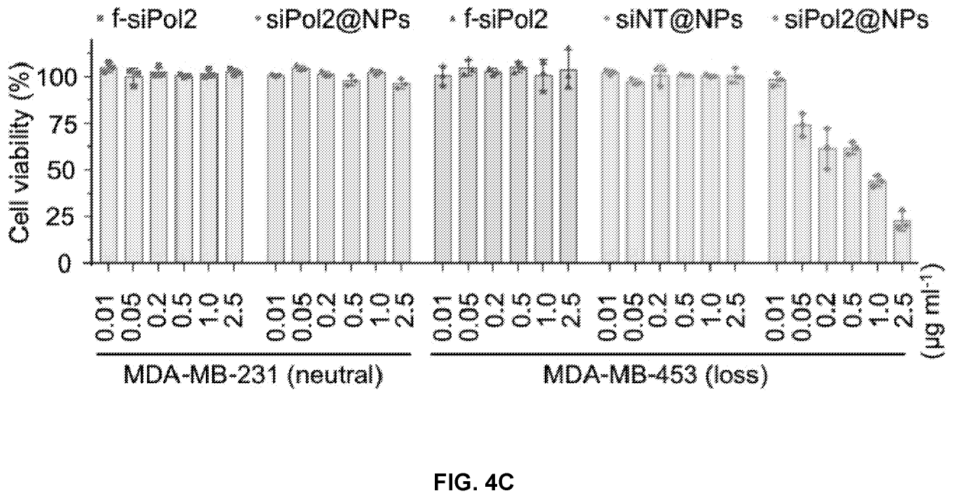

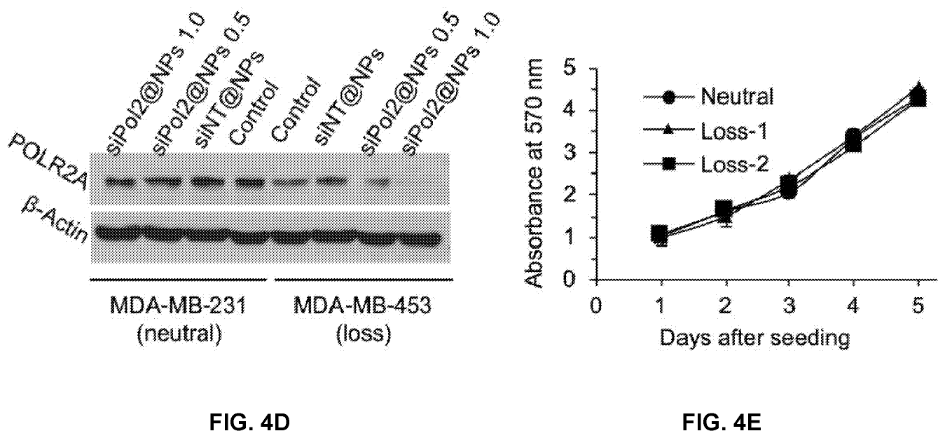

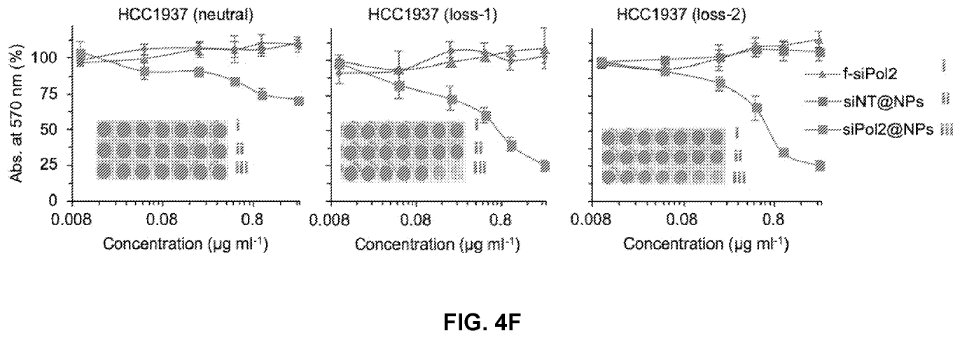

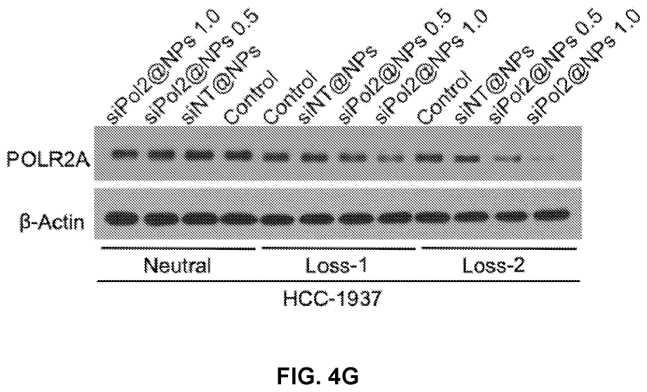

The POLR2A targeted strategy inspired by the data shown in FIG. 1 for killing TNBC cells is illustrated in FIG. 4A. To investigate the sensitivity of POLR2A^{loss} TNBC cells to the POLR2A inhibition, MDA-MB-453 (TP53^{-/mut}, POLR2A^{-/+}, or POLR2A^{loss}) and MDA-MB-231 (TP53^{+/mut}, POLR2A^{+/+}, or POLR2A^{neutral}) breast cancer cells were treated with siPol2@NPs together with control conditions. As shown in FIG. 4B for the colony formation assay, siNT@NPs (nanoparticles with non-targeting siRNA) is not harmful to MDA-MB-453 cells, which is expected as the nanoparticle formulation contains FDA-approved biocompatible materials. This also indicates the “nano-bomb” effect of the nanoparticles alone is not harmful to cells. The free siPol2 did not show any cell-killing effect either, due to its instability in serum and poor uptake by cells. In contrast, the siPol2@NPs could kill the MDA-MB-453 cells (POLR2A^{loss}) in a concentration-dependent manner although their toxicity to MDA-MB-231 cells (POLR2A^{neutral}) is negligible. These observations are confirmed by the cell counting kit-8 (CCK-8) assay (FIG. 4C). These results indicate the POLR2A^{loss} MDA-MB-453 cells are more sensitive to siPol2@NPs than POLR2A^{neutral} MDA-MB-231 cells, presumably because the partial loss of POLR2A renders MDA-MB-453 cells highly vulnerable to POLR2A inhibition. This is confirmed by the POLR2A protein level in the two cells after treatments (FIG. 4D). The siPol2@NPs could effectively minimize POLR2A expression in POLR2A^{loss} cells at a siPol2 concentration of 1.0 μg ml⁻¹. POLR2A expression in POLR2A^{neutral} cells treated with the 1.0 μg ml⁻¹ encapsulated siPol2 is still evident albeit decreased. Therefore, significant inhibition of POLR2A expression is selectively lethal to POLR2A^{loss} TNBC cells.

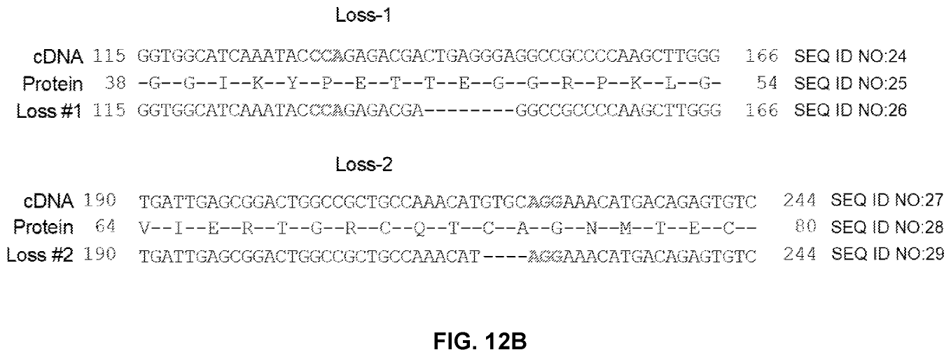

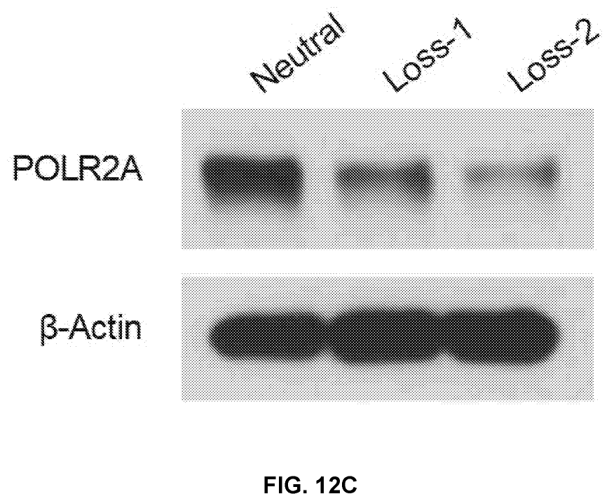

To exclude potential genetic differences across cell lines, two isogenic HCC1937 cell lines were generated with hemizygous loss of POLR2A using CRISPR (clustered regularly interspaced short palindromic repeat)/Cas9 (FIGS. 12A, 12B). The wild type HCC1937 is TP53^{+/mut}, POLR2A^{+/+} (POLR2A^{neutral}). The parent and two isogenic POLR2A^{loss} HCC1937 cells exhibited similar cell growth rates (FIG. 4E), confirming hemizygous deletion of POLR2A does not affect cell survival. However, hemizygous loss of POLR2A (HCC1937^{loss-1} and HCC1937^{loss-2}) markedly sensitizes the HCC1937 cells to siPol2@NPs (FIG. 4F). As expected, siNT@NPs or free siPol2 had no substantial effect on the cell proliferation. POLR2A expression is reduced in isogenic HCC1937 cell lines (FIGS. 4G, 12C). Furthermore, siPol2@NPs decreases POLR2A expression in all the cells. Collectively, these data show the materials in the nanoparticles (other than siPol2) do not have an effect on POLR2A expression in cells or their proliferation, and siPol2@NPs effectively kill POLR2A^{loss} isogenic TNBC cells.

Suppression of POLR2A Inhibits POLR2A^{loss} Tumour Growth

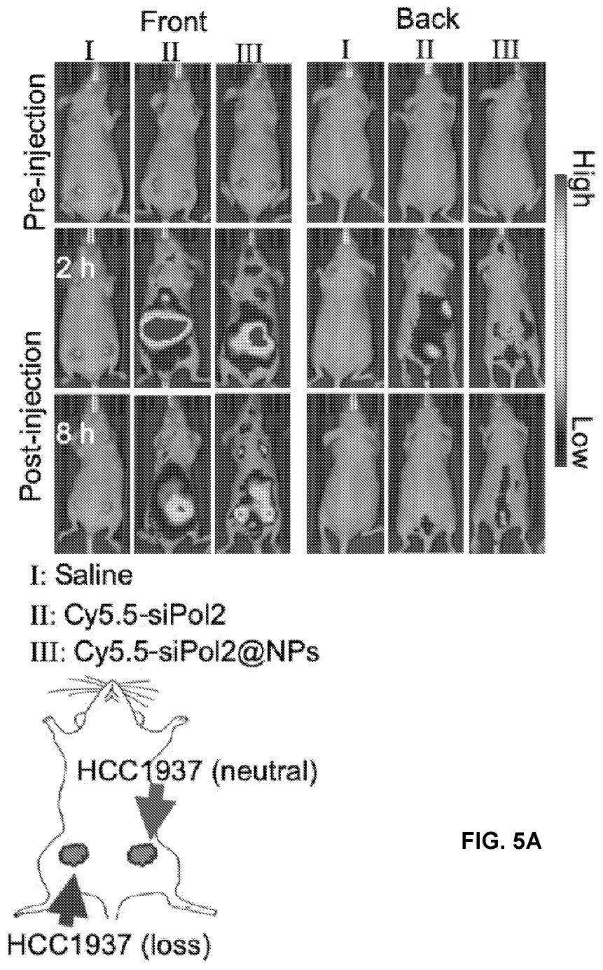

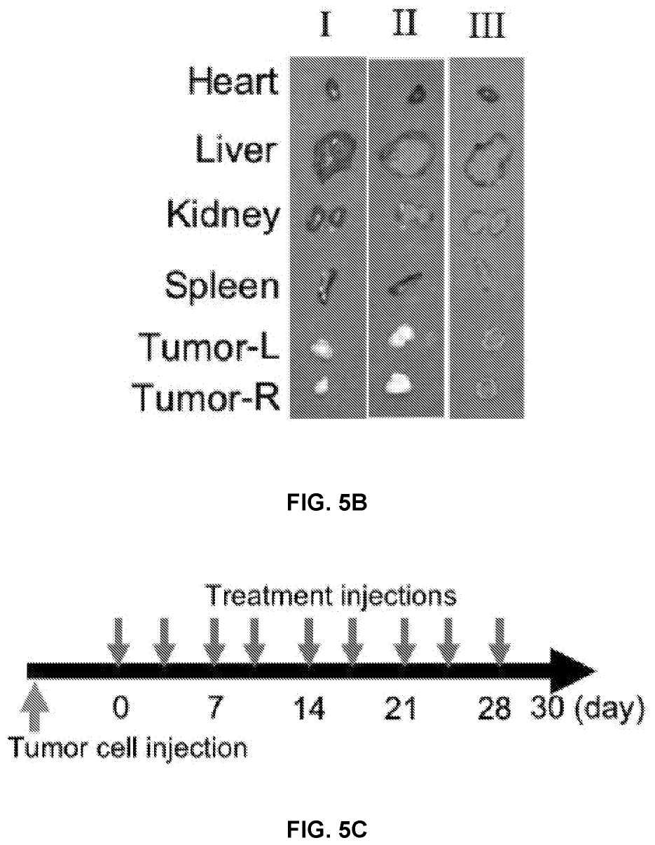

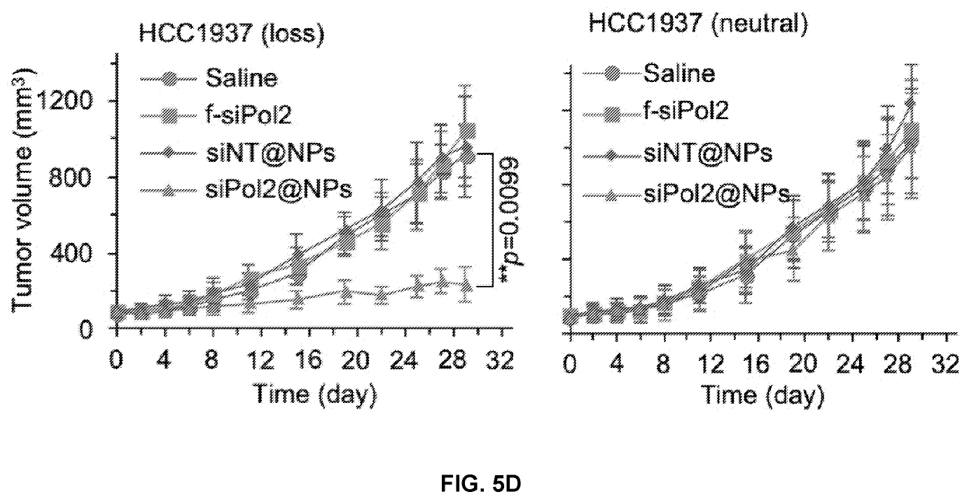

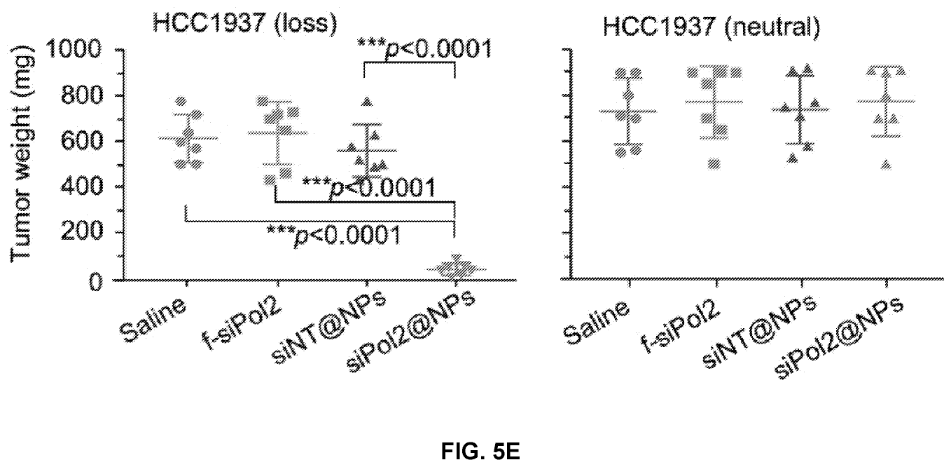

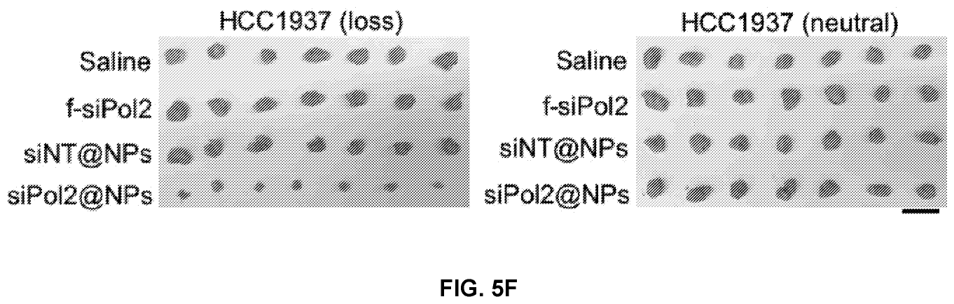

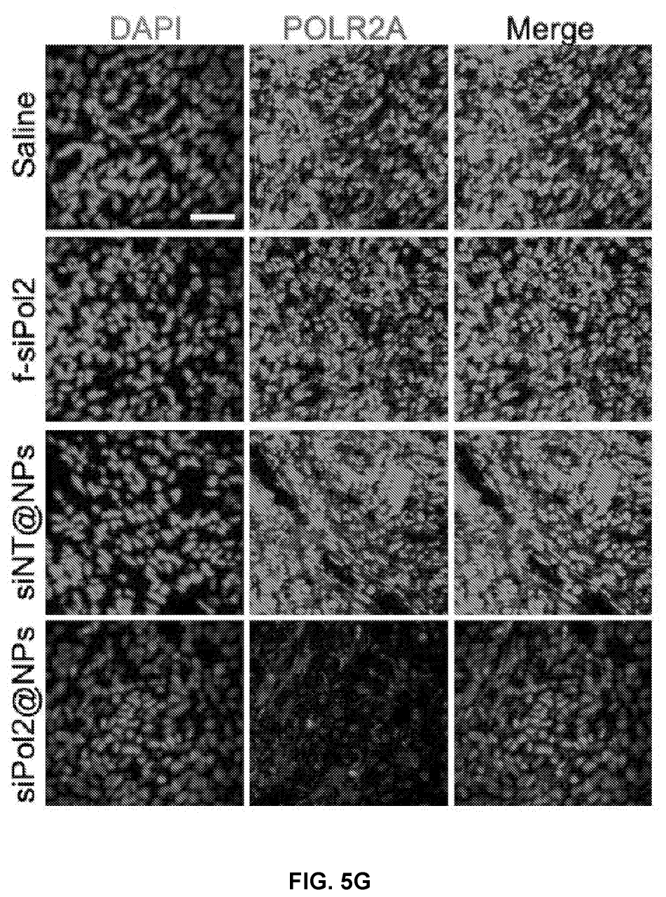

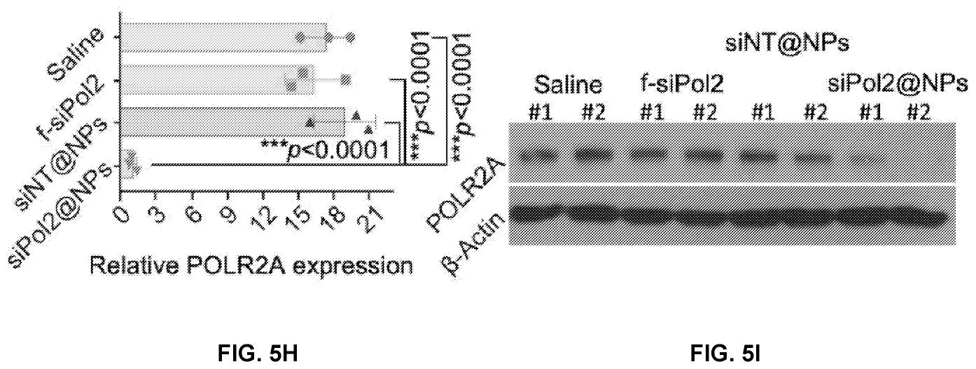

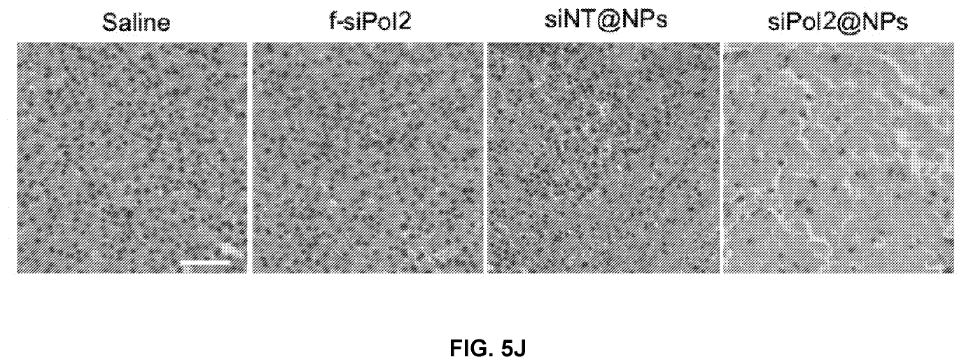

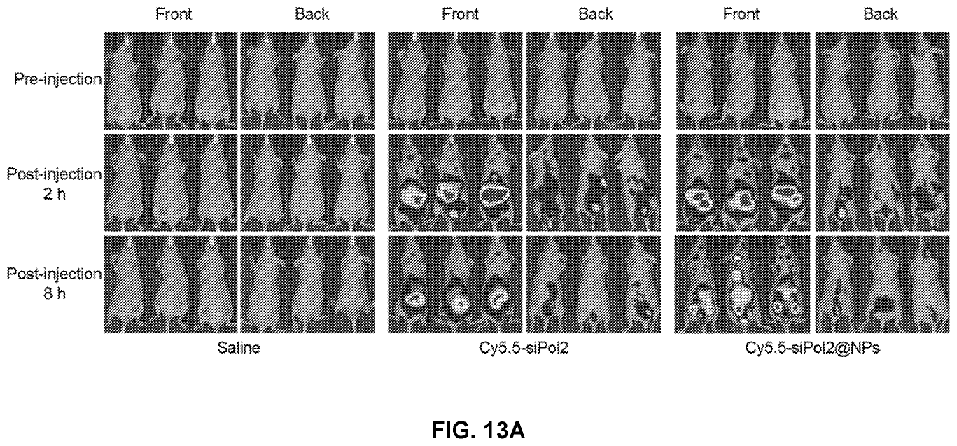

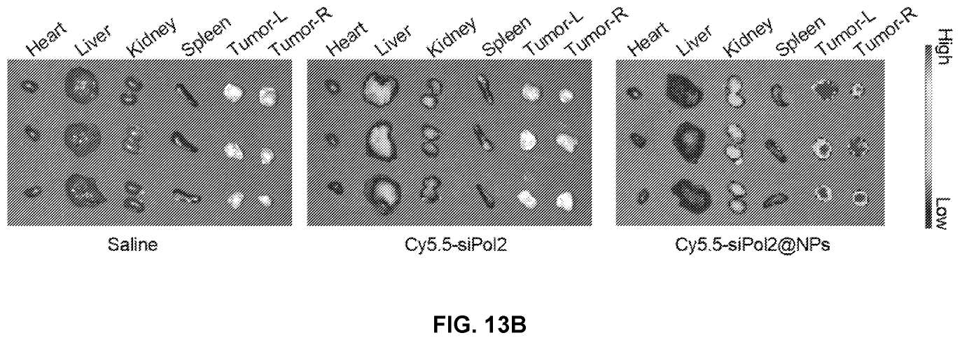

To establish orthotopic HCC1937 TNBC tumours, the isogenic POLR2A^{loss} and wild type POLR2A^{neutral} HCC1937 cells were injected into the 4^{th} inguinal mammary fat pad on the left and right sides of each mouse (FIG. 5A). For examining the tumour targeting capability of the nanoparticles, siPol2 labelled with Cy5.5 was used to form Cy5.5-siPol2@NPs for in vivo imagining. As shown in FIGS. 5A, 13, highly enhanced fluorescence signal is observable in tumours at 8 h after administration of the Cy5.5-siPol2@NPs compared to free Cy5.5-siPol2, probably due to the enhanced permeability and retention (EPR) effect of tumour vasculature (Farokhzad, O C, et al. ACS Nano 2009 3(1):16-20; Wilhelm, S, et al. Nat. Rev. Mater. 2016 1:16014). This observation was confirmed by ex vivo imaging of five major organs along with tumours harvested from the mice sacrificed at 8 h (FIGS. 5B, 13). On one hand, only tumours from the Cy5.5-siPol2@NPs group present strong signal of Cy5.5, compared with tumours from the saline or free Cy5.5-siPol2 group. On the other hand, in the Cy5.5-siPol2@NPs group, the isogenic POLR2A^{loss} tumours on the left have similar fluorescence intensity to the wild type POLR2A^{neutral} tumours on the right. This indicates hemizygous loss of POLR2A does not affect the accumulation of nanoparticles in vivo, excluding the possibility that an enhanced therapeutic effect on the POLR2A^{loss} tumour is due to improved agent accumulation in the tumours. No accumulation of the free or encapsulated Cy5.5-siPol2 is observable in heart or kidney. Weaker fluorescence was observable in livers from the free Cy5.5-siPol2 or Cy5.5-siPol2@NPs groups. This is expected because the agents are probably cleared out of the body through liver.

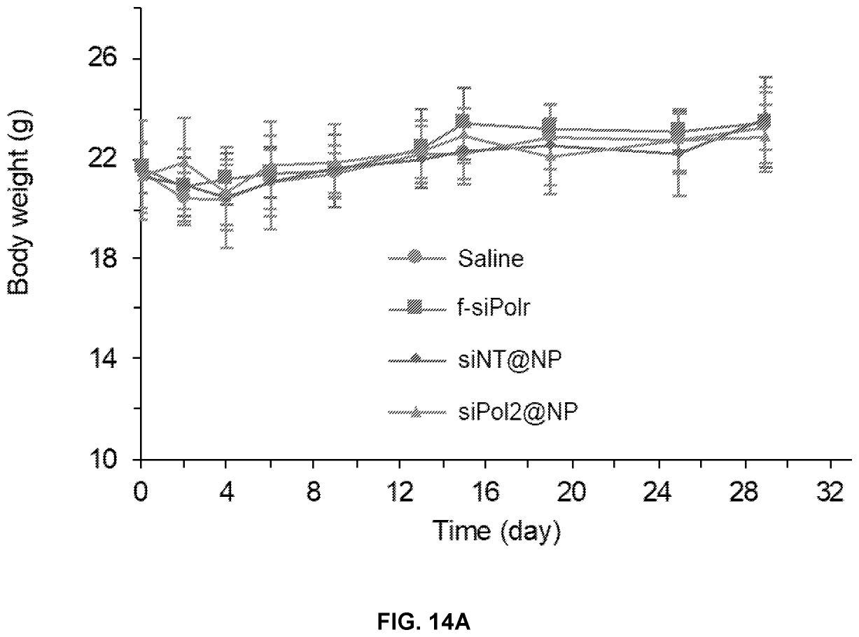

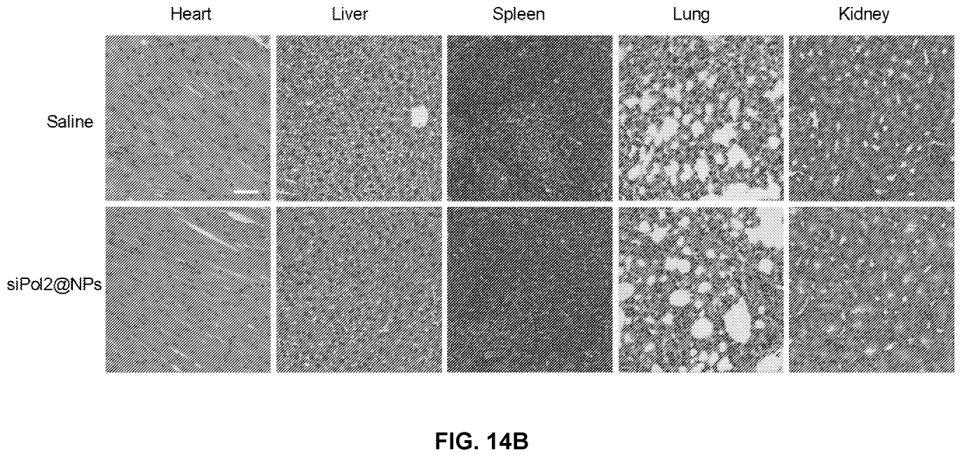

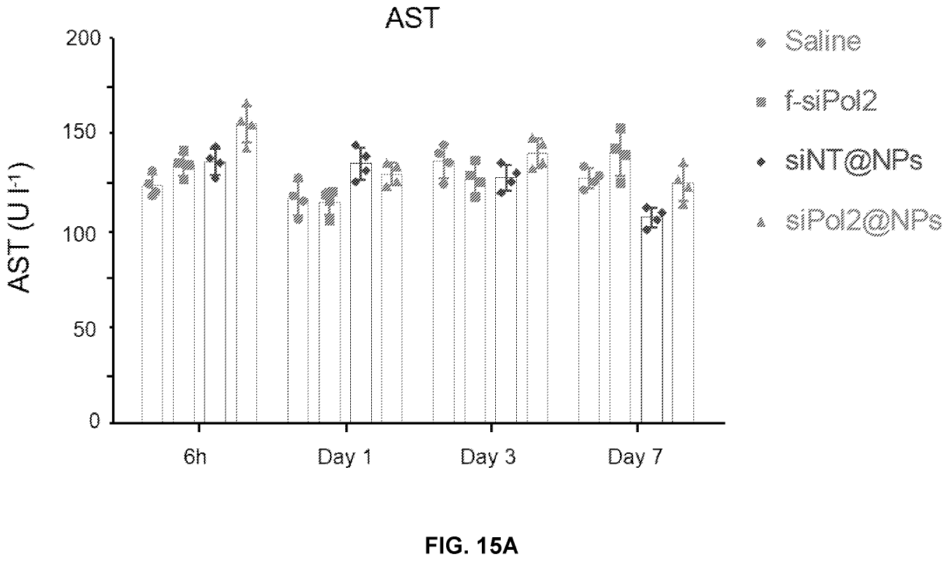

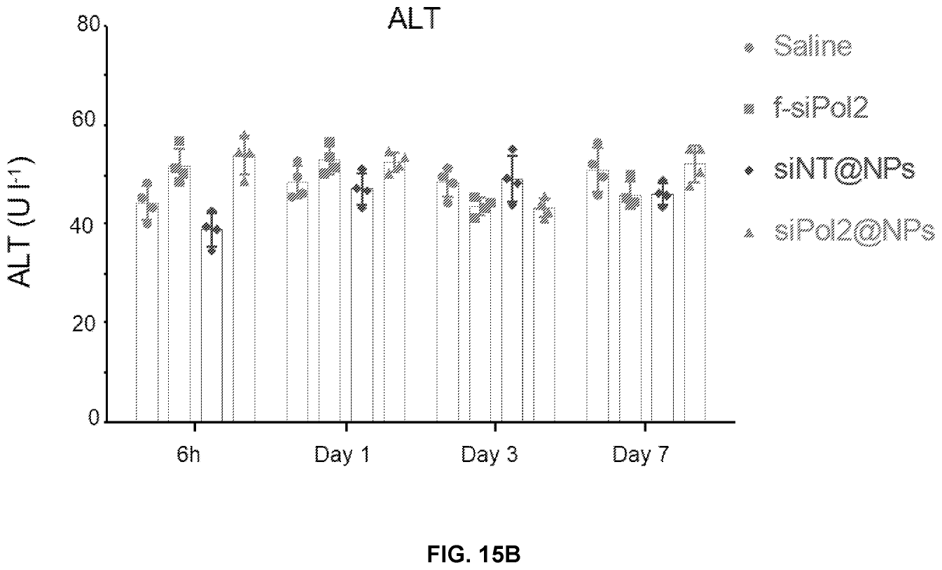

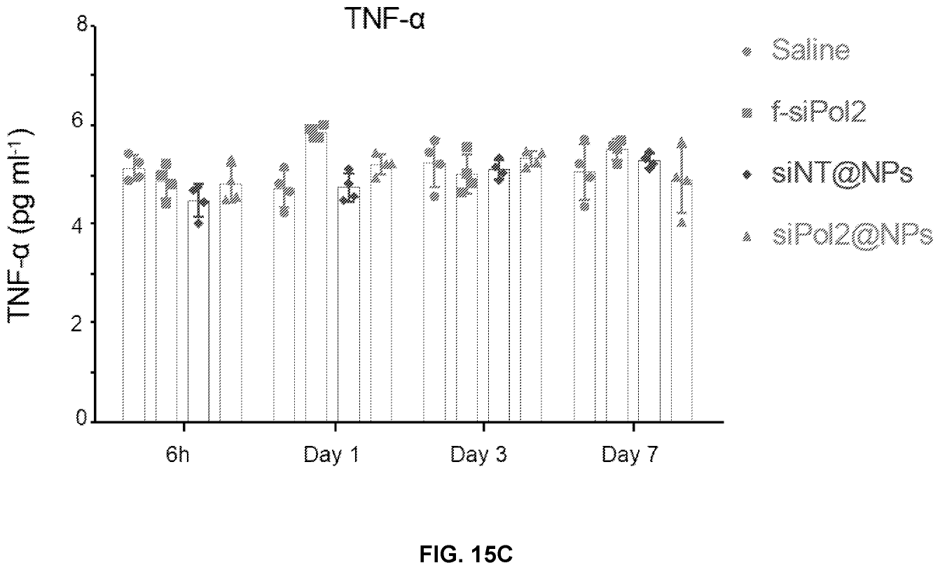

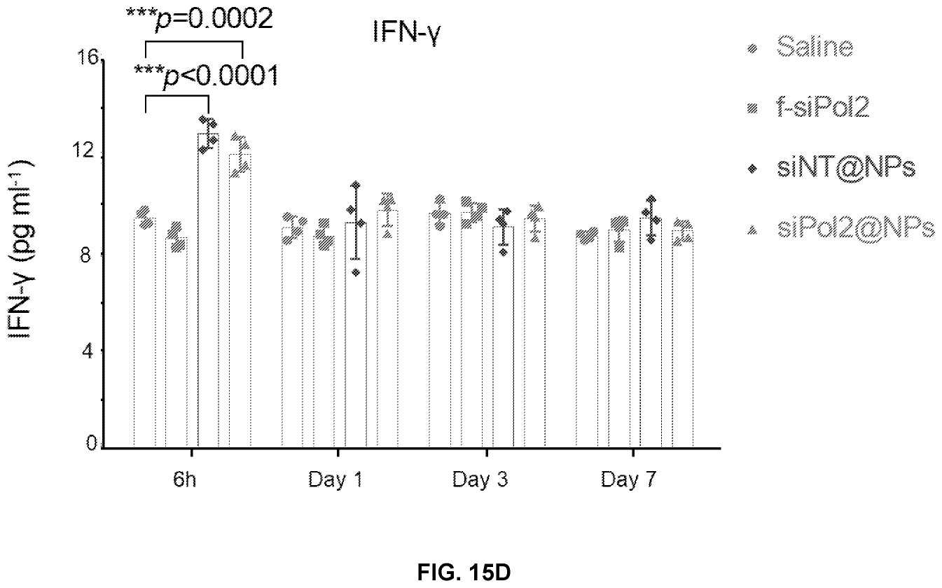

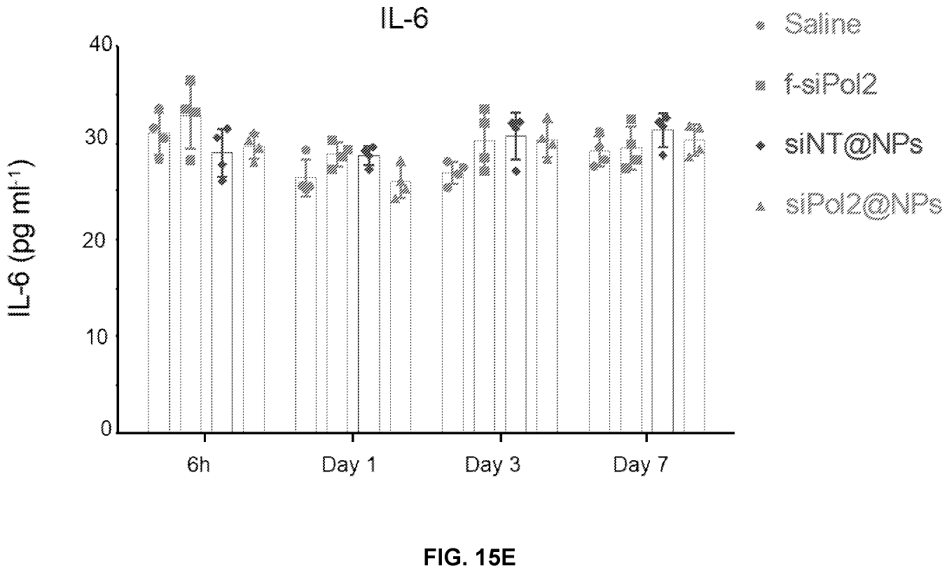

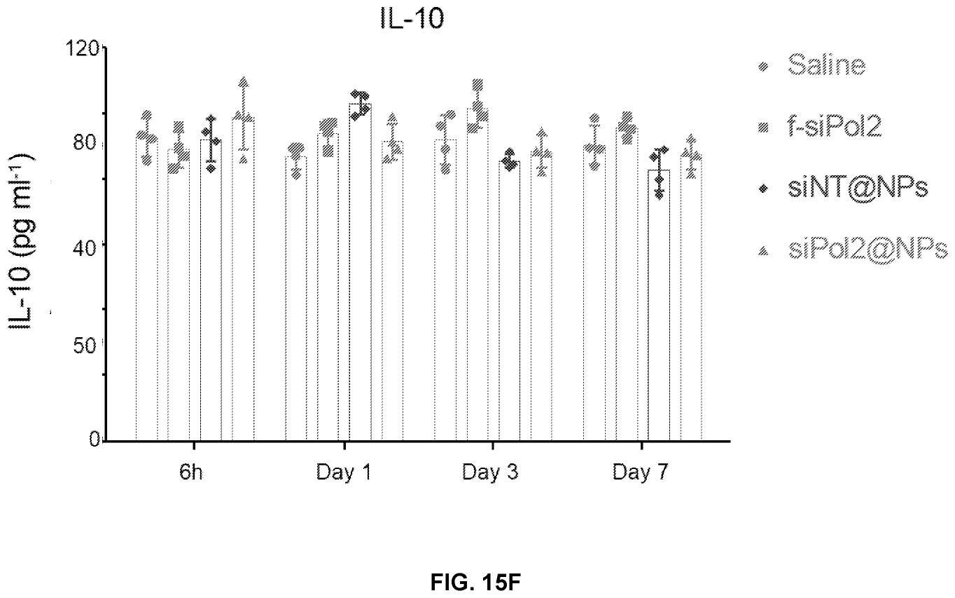

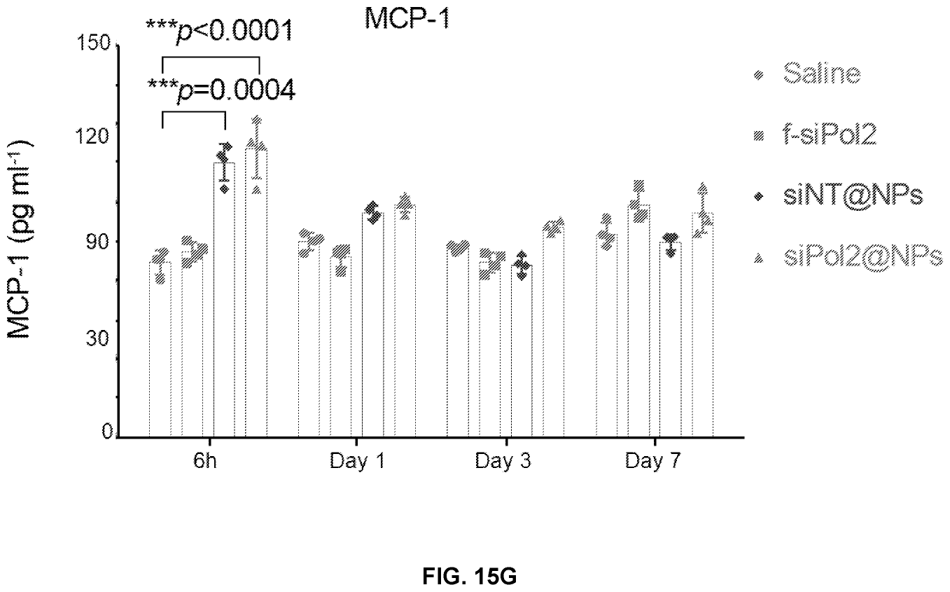

To investigate the material safety and antitumor efficacy, four different treatments (saline, free siPol2, siNT@NPs, and siPol2@NPs) were applied twice a week after tumour establishment in nude mice (FIG. 5C). Neither mouse death nor significant drop in body weight was observed in all the groups during the experiments (FIG. 14A). Furthermore, no obvious damage is observable in the haematoxylin and eosin-stained histology slices of five major organs collected from mice in the siPol2@NPs and saline groups (FIG. 14B). Moreover, no obvious change in aspartate aminotransferase and alanine aminotransferase levels is observable in wild type C57BL/6J mice with intact immune system after treated with the various formulations (FIG. 15), indicating that the nanoparticles are well tolerated by the animal. The serum levels of tumour necrosis factor-α, Interferon-γ, Interleukin-6, Interleukin-10, and monocyte chemoattractant protein-1 were further measured in the wild type mice. Although significantly increased levels of Interferon-γ and monocyte chemoattractant protein-1 were observed for the siNT@NP and siPol2@NP treatments at 6 h after injection, they returned to the baseline levels on day 1 and thereafter. No significant difference was observed between the various conditions for all the other cytokines in 7 days, indicating negligible immune response induction by the nanoparticles.

Inhibiting POLR2A by siPol2@NPs significantly inhibited the POLR2A^{loss} tumour growth by ˜80% (left panels in FIG. 5D-5F) compared with the tumour from control (saline) group. By contrast, no significant antitumor effect was observed on the POLR2A^{neutral} tumours (right panels in FIG. 5D-5F). As expected, the POLR2A protein levels in siPol2@NPs treatment group were minimized (FIG. 5G-5I). These data confirm that POLR2A silencing leads to selective suppression of POLR2A^{loss} tumours. Moreover, histological staining reveals extensive necrosis in the POLR2A's tumours only from the siPol2@NPs group (FIG. 5J).

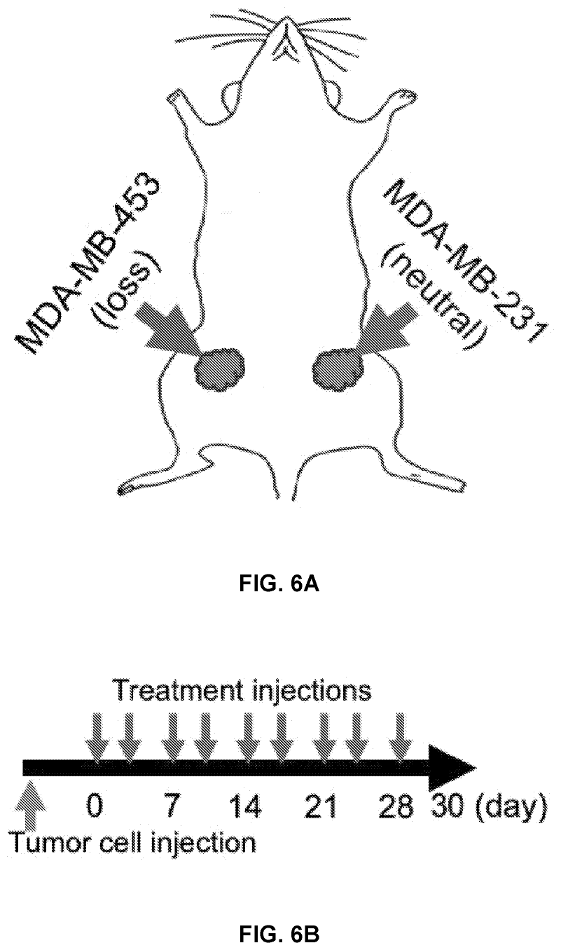

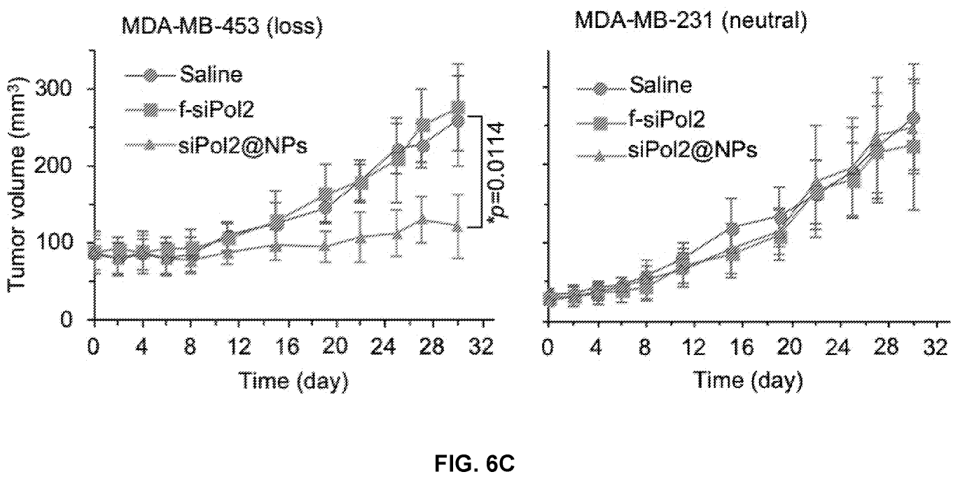

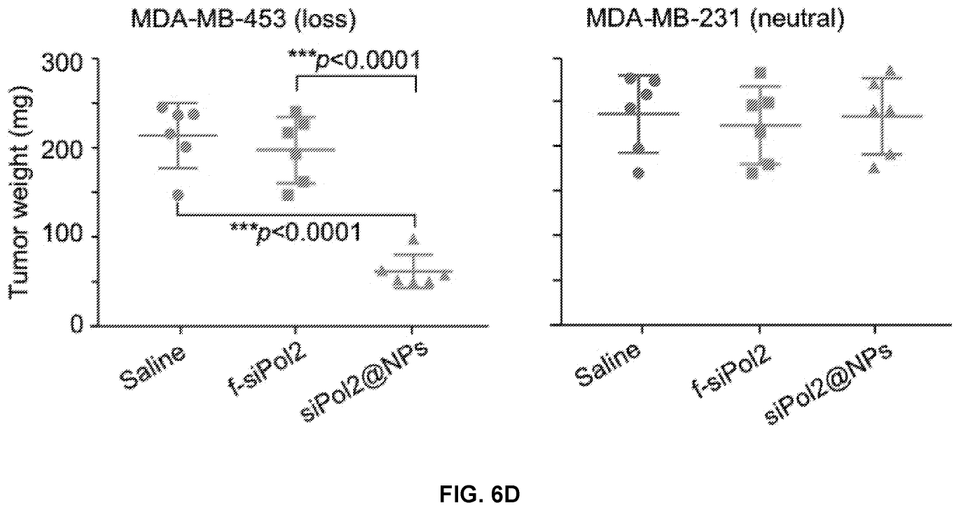

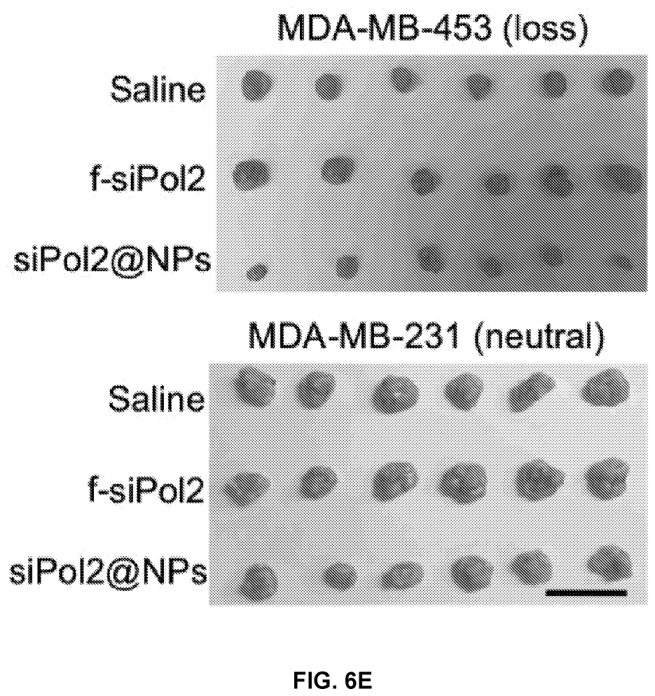

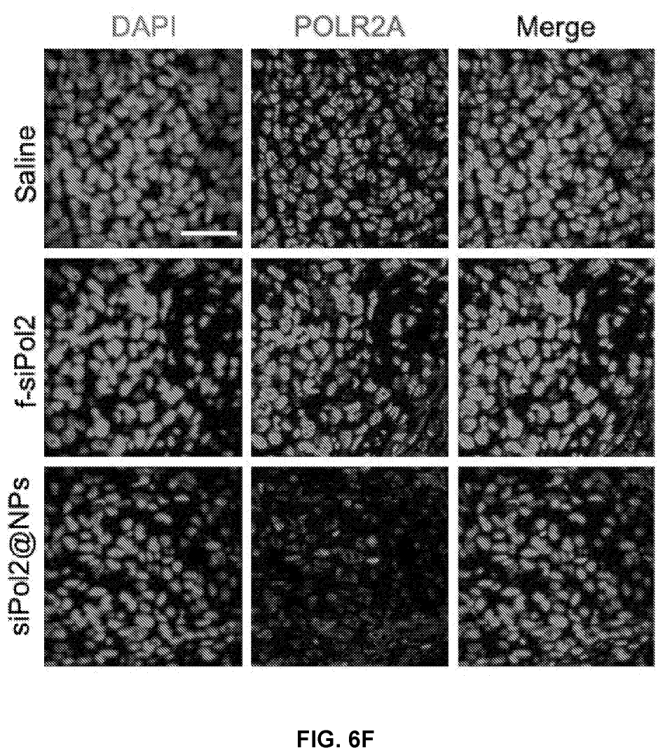

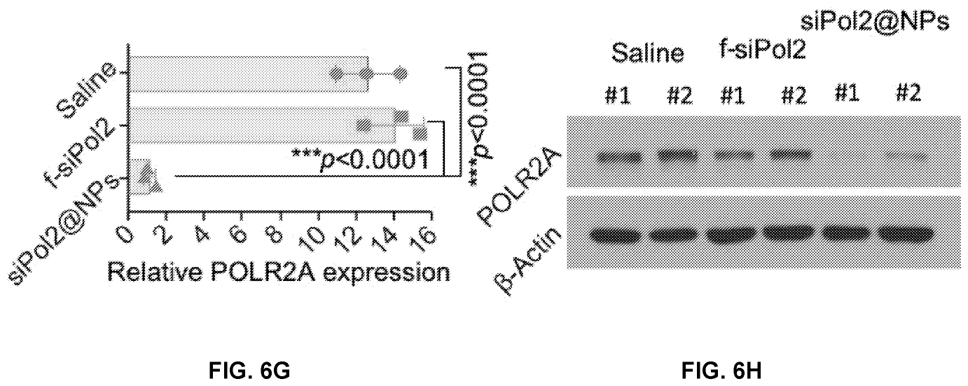

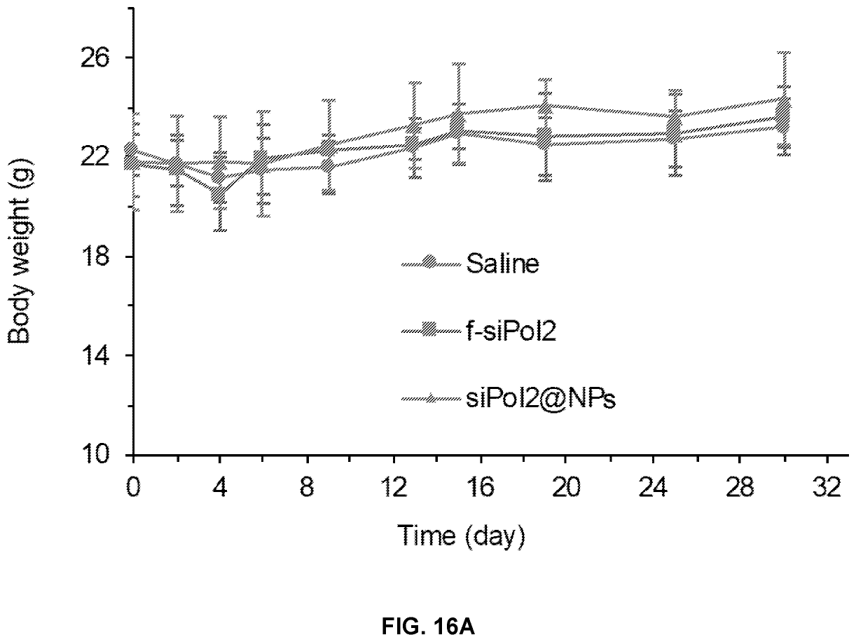

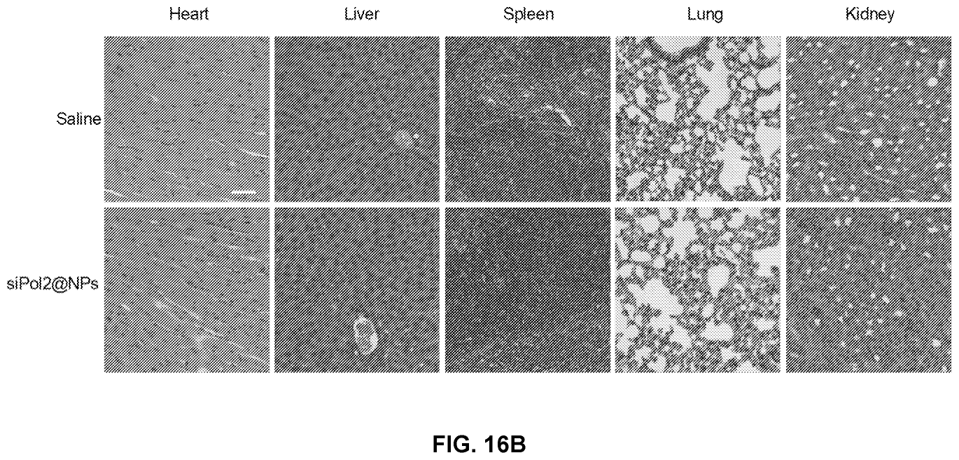

To confirm the findings with tumours derived from the isogenic pairs of HCC1937 cells, wild type POLR2A^{loss} (MDA-MB-453) and POLR2A^{neutral} (MDA-MB-231) cells were used to establish the orthotopic tumours and did treatment injections of saline, siNT@NPs, and siPol2@NPs twice a week (FIG. 6A-6B). Neither mouse death nor significant body weight drop was observed in all the groups (FIG. 16A), and no evident damage could be observed in all the five organs from in the siPol2@NPs treatment group (FIG. 16B). Furthermore, siPol2@NPs exhibit excellent antitumor capacity to MDA-MB-453 (POLR2A^{loss}) tumours, while no significant difference in the growth of the MDA-MB-231 (POLR2A^{neutral}) tumours is observable for the three different groups (FIG. 6C-6E). The siPol2@NPs treatment minimizes the POLR2A protein levels in the MDA-MB-453 tumours (FIG. 6F-6H), confirming that POLR2A silencing leads to tumour suppression in the POLR2A^{loss} tumour model. Histological staining shows extensive necrosis in the POLR2A^{loss} tumours only from the siPol2@NPs group (FIG. 6I).

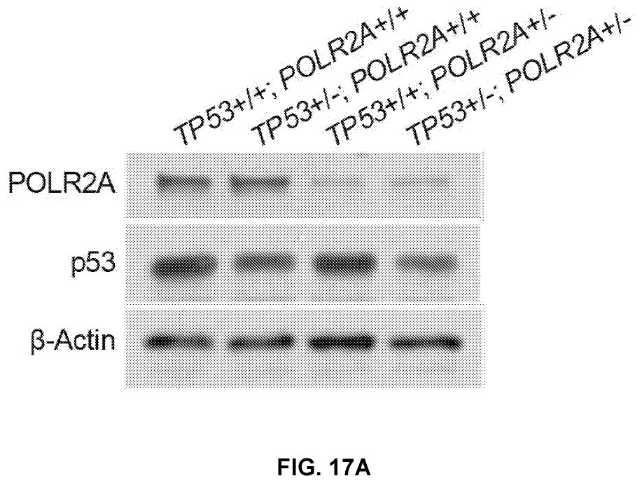

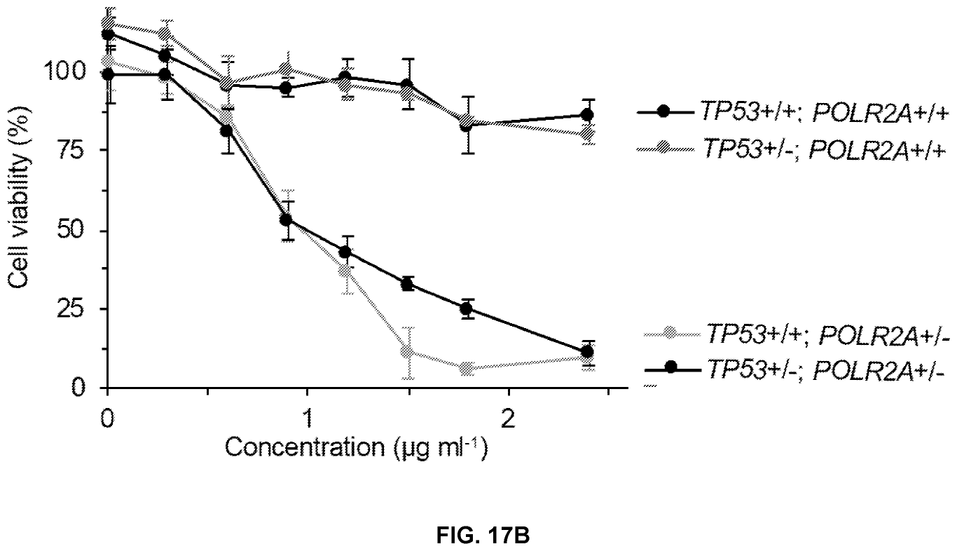

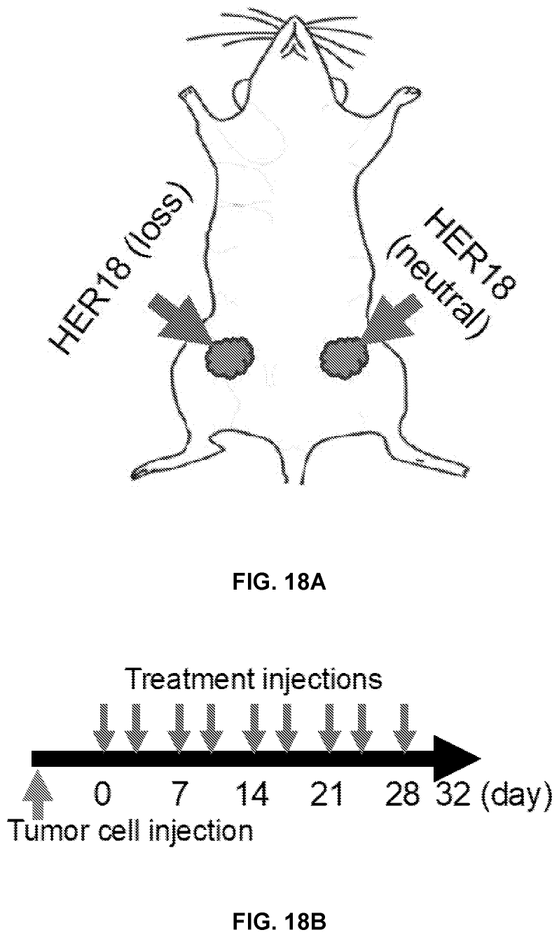

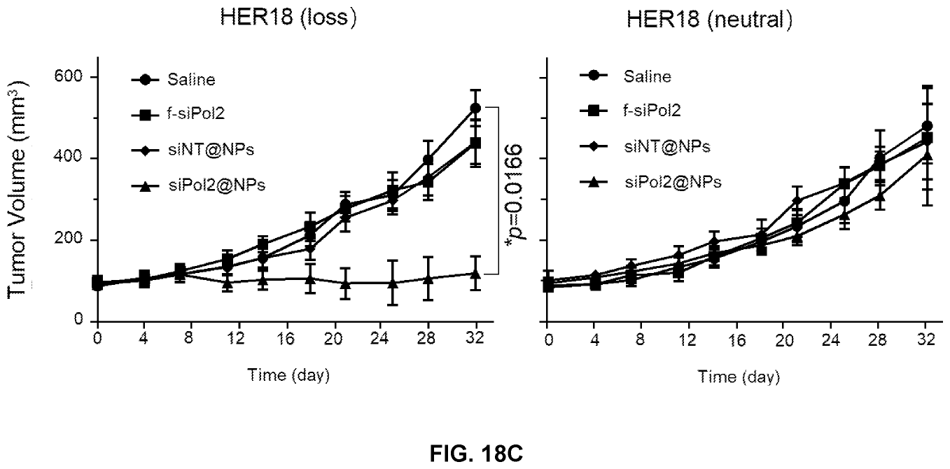

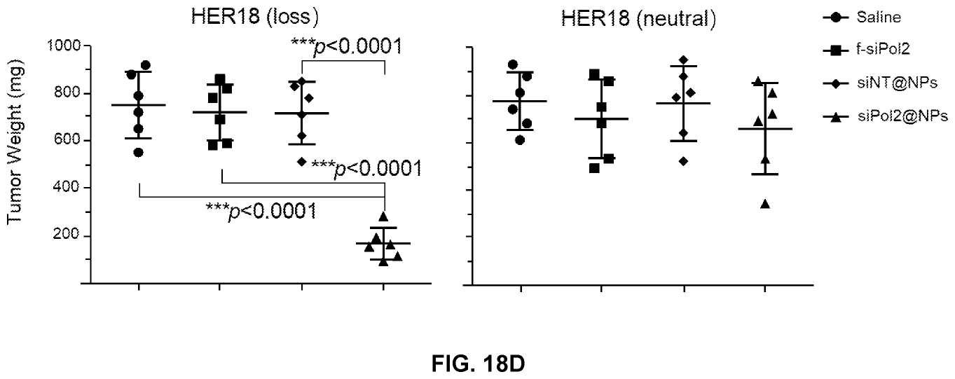

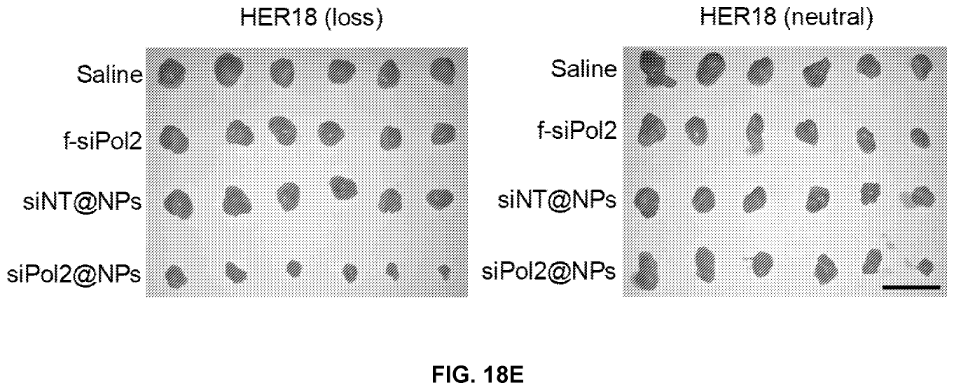

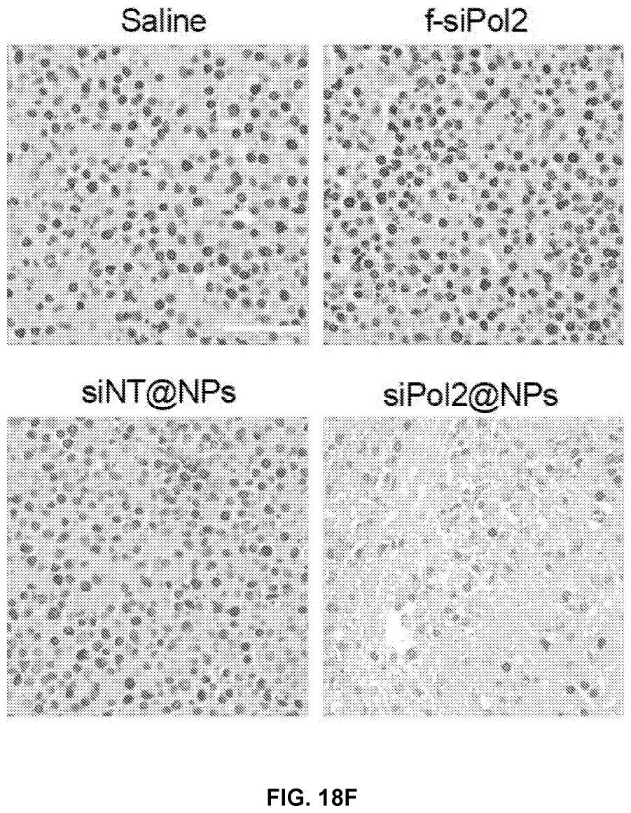

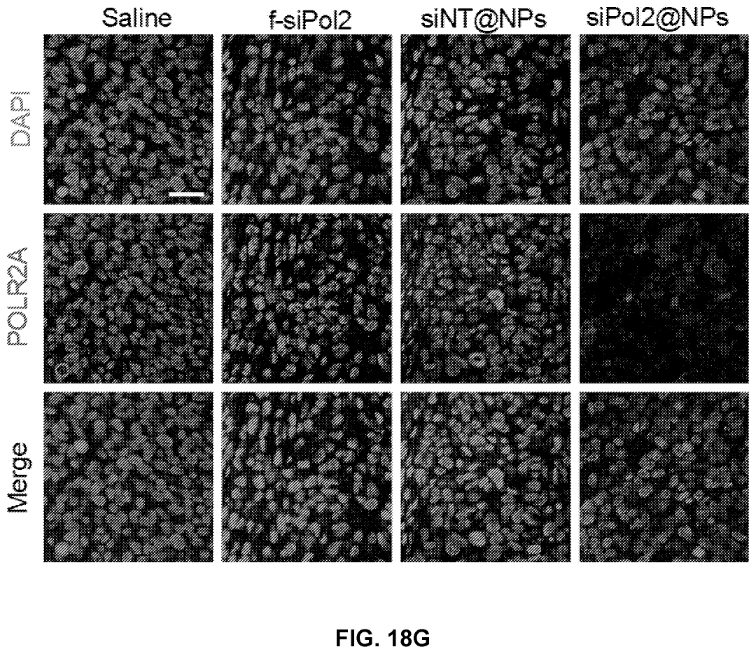

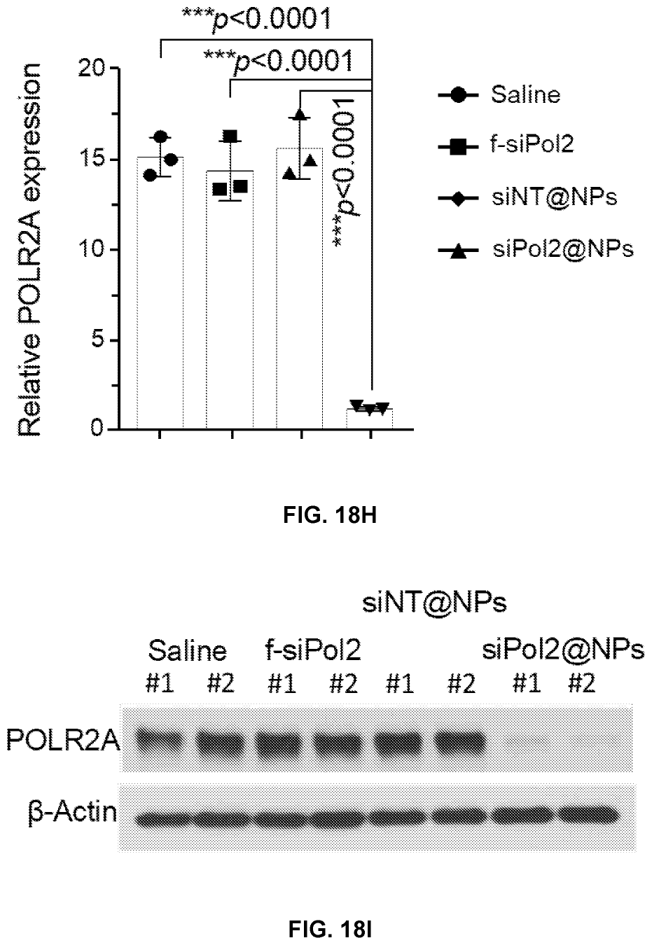

It is worth noting that the strategy of targeting POLR2A using the siPol2@NPs can also be used to selectively inhibit the growth of POLR2A^{loss} HER2+ HER18 breast cancer cells (in vitro) and tumours (in vivo) (FIGS. 17-18). Altogether, the siPol2@NPs based POLR2A suppression inhibits the growth of POLR2A^{loss} MDA-MB-453 (TP53^{-/mut}, POLR2A^{-/+}), isogenic HCC1937 (TP53^{mut/+}, POLR2A^{+/-}), and isogenic HER18 (TP53^{+/+}, POLR2A^{+/-} and TP53^{+/-}, POLR2A^{+/-}) cells and tumours, regardless of their TP53 status. While TP53 and POLR2A are often co-deleted in a majority of human cancers with hemizygous loss of TP53, the status of TP53 has no significant impact on the anti-cancer activity of POLR2A inhibition.

As shown in FIG. 10, hemizygous loss of TP53 occurs in approximately 75% of HER2 positive (HER2+) breast cancer. Even though there are a variety of approved therapies for treating HER2+ breast cancer, acquired resistance to its therapy remains universal and approaches are still in need to address it. Therefore, the potential applications of using siPol2@NPs to precisely target POLR2A in HER2+ subtype breast cancer was further investigated. To further confirm the hypothesis that POLR2A targeted therapy is independent of the TP53 status, three isogenic HER18 cell lines of HER2+ breast cancer (TP53+/−, POLR2A+/+; TP53+/+, POLR2A+/−; TP53+/−, POLR2A+/−) were generated using the CRISPR/Cas9 technology (FIG. 17A) from the parent HER18 cells (TP53+/+, POLR2A+/+). As shown in FIG. 17B, the two HER18 cell lines with POLR2Aloss (TP53+/+, POLR2A+/− and TP53+/−, POLR2A+/−) are significantly more sensitive to the POLR2A knockdown in comparison with the other two HER18 cell lines with POLR2A^{neutral} (TP53+/+, POLR2A+/+ and TP53+/−, POLR2A+/+). To test the strategy for treating HER2+ human breast cancer in vivo, HER2+ breast tumours were established in nude mice using the isogenic HER18 cells (TP53+/+, POLR2A+/−) and the parent HER18 cells (TP53+/+, POLR2A+/+) as illustrated in FIG. 18A-18B. Inhibiting POLR2A by siPol2@NPs significantly reduced the POLR2A^{loss} tumour growth by ˜75% (FIG. 18C-18E) in all six mice compared to the tumour from the control (saline) group. POLR2A protein levels in the siPol2@NPs treatment group were minimized (FIG. 18F-18I). This observation in the HER2+ tumour model is consistent with that observed in the TNBC mouse models. These results confirm that further POLR2A silencing in cancer cells containing hemizygous TP53 deletion leads to tumour suppression, and the POLR2A targeted therapy with siPol2@NPs is a promising therapeutic approach for both TNBC and HER2+ breast cancer and possibly other breast cancer subtypes.

Conclusions

This study, by analysing TNBC databases, reveals that POLR2A gene is almost always co-deleted with TP53 in the Chr17p deletion region, and 53% of TNBC harbours this heterozygous deletion. Moreover, the POLR2A expression levels are highly correlated with the copy number of POLR2A, rendering cancer cells with heterozygous loss of TP53 vulnerable to POLR2A inhibition. This collateral loss of POLR2A with TP53 prompted us to use RNAi to precisely target POLR2A for TNBC treatment. To overcome the hurdles to cytosolic delivery of siRNA for RNAi, a unique nanoplatform with a low pH-activated “bomb-like” effect for endo/lysosomal escape was developed. This improves cytosolic delivery of siPol2 to inhibit POLR2A in TP53^{loss} cells. The anticancer capability of siPol2@NPs in vivo was examined using three different orthotopic tumour models derived from both wild type and isogenic cell lines. The nanoparticles were found to preferentially accumulate in both the POLR2A^{neutral} and POLR2A^{loss} tumours. Importantly, the data show that, in tumours with heterozygous POLR2A loss, inhibition of POLR2A with siPol2-laden nanoparticles leads to an enhanced reduction of the tumour growth with no evident systemic toxicity. This approach also possesses several important advantages for clinical applications. Collectively, this study may provide a promising nanotechnology-based precision-targeting strategy for fighting against TNBC and potentially many other types of cancers harbouring the common TP53 genomic alteration, regardless of the TP53 status.

The hemizygous deletion of TP53, which often involves a large fragment (over several megabases), even the whole short arm of chromosome 17 (17p), is a frequent genomic event across many types of human cancers. In tumours with hemizygous loss of TP53, POLR2A is almost always co-deleted. As an example, 99.5% (575 out of 578) of human breast cancers with hemizygous loss of TP53 contain co-deletion of POLR2A. In clinical practice, the copy number of TP53 gene has been examined by fluorescence in situ hybridization (FISH). The use of archival frozen tumour tissue imprint specimens for FISH has been well established in the clinic. Quantitative PCR and SNP DNA microarrays are also feasible to detect copy number changes of TP53 and POLR2A. Based on the extensive cancer genomics data, it appears to be unnecessary to check the heterozygous deletion of POLR2A in the cancers harbouring hemizygous loss of TP53. Approximately half of human breast cancers with hemizygous loss of TP53 harbour mutant TP53 on the remaining allele, in support of the two-hit hypothesis in human cancer. However, the sensitivity of cancer cells to POLR2A inhibition appears to be primarily dependent on the status of POLR2A, regardless of TP53 status. Moreover, given the prevalence of TP53 loss in all breast cancer subtypes as well as other types of human cancer, the principle of essential lethality to POLR2A inhibition can also be applied to other human cancers including HER2+ breast cancer. Over the last a few years, the rapid development of high throughput platforms such as microarrays and next generation sequencing technologies offers a promising prospect for the translation of the POLR2A-targeted therapy.

The knowledge and arsenal of cancer nanomedicine have rapidly expanded in the past several years (Shi, J, et al. Nat. Rev. Cancer 2016 17:20). However, only few nanoparticle-based RNAi therapeutics have entered the clinical trial phase (Shi, J, et al. Nat. Rev. Cancer 2016 17:20; Wittrup, A, et al. Nat. Rev. Genet. 2015 16:543; Bobbin, M. L, et al. Annu. Rev. Pharmacol. Toxicol. 2016 56(1):103-122; Dahlman, J. E, et al. Nat. Nanotechnol. 2014 9:648). Lipid nanoparticles or liposomes-based delivery of siRNA is the most investigated approach in clinical trials (Blanco, E, el. Nat. Biotechnol. 2015 33:941). Unfortunately, this approach has not reached phase II/III stages of clinical trial. One of the major reasons is that it cannot achieve endo/lysosomal escape to allow the siRNA to reach the RNA-Induced Silencing Complex (RISC) located in the cytoplasm. Endo/lysosomal escape of siRNA is essential because the RNases inside the endo/lysosomes could quickly degrade the therapeutic agent before it can reach its target. Unlike the liposomes-based approach, the disclosed pH-activated “nano-bomb” nanoparticles were designed for enhanced cytosolic delivery of siRNA because it can respond to the low-pH environment in endo/lysosomes to quickly release most of the encapsulated siRNA into the cytosol before it is degraded. This leads to a highly efficient utilization of the siRNA, which promotes high therapeutic efficacy with minimized siRNA dose compared to the liposomes-based approach. Moreover, tumour cells harbouring hemizygous deletion of TP53, which is a common genetic alteration in cancer, are markedly sensitive to further POLR2A inhibition. In other words, low dosage of siPol2@NPs could be used to kill POLR2A^{loss} cells (tumour cells) but not POLR2A^{neutral} cells (healthy cells). Another major reason causing nanomedicine to fail in clinical trial (phase I) is the undesired side effects (Bobbin, M. L, et al. Annu. Rev. Pharmacol. Toxicol. 2016 56(1):103-122; Blanco, E, el. Nat. Biotechnol. 2015 33:941). The disclosed nanoparticles were synthesized using FDA-approved biocompatible materials, which should minimize the undesired side effects. Therefore, the siPol2@NPs capable of quickly escaping endo/lysosomes triggered by low pH and precisely targeting POLR2A^{loss} cancer cells have tremendous potential for effective and safe delivery of siRNA to treat patients with cancers harbouring hemizygous loss of POLR2A regardless of the TP53 status.

### Example 2: Targeting 17q23 Amplicon to Overcome the Resistance to Anti-HER2 Therapy in HER2+ Breast Cancer

Multiple trastuzumab-resistance mechanisms have been identified in preclinical studies, in which constitutive activation of the PI3K pathway owing to PTEN deficiency or PIK3CA mutations seems to be one of the most prevalent events (Nagata Y, et al. Cancer Cell 2004 6:117-127; Zhang S, et al. Nat Med 2011 17:461-469; Liang K, et al. Mol Cancer Ther 2003 2:1113-1120). The better understanding of breast cancer biology has translated into the development of novel anti-HER2 agents with varying mechanisms of action (Stern H M. Sci Transl Med 2012 4:127rv122; Zardavas D, et al. Curr Opin Oncol 2012 24:612-622). The small molecular tyrosine kinase inhibitor lapatinib has demonstrated activity in HER2+ metastatic breast cancer and in the preoperative setting (Geyer C E, et al. N Engl J Med 2006 355:2733-2743; Baselga J, et al. Nat Rev Cancer 2009 9:463-475). Pertuzumab, a monoclonal antibody with a distinct binding site from trastuzumab, inhibits receptor dimerization (Franklin M C, et al. Cancer Cell 2004 5:317-328; Swain S M, et al. N Engl J Med 2015 372:724-734). The addition of pertuzumab to combination therapy has led to improvements in progression-free survival in patients with HER2+ metastatic breast cancer and higher response rates in the preoperative setting (Swain S M, et al. N Engl J Med 2015 372:724-734).

To maintain genome stability, eukaryotic cells have evolved with the ability to detect and translate the initial signals of DNA damage to proper cellular responses. The key components of the DNA damage response (DDR) are the phosphoinositide-3-kinase-related kinase (PIKK) family, which includes ATM (ataxia-telangiectasia mutated), ATR (ataxia-telangiectasia and Rad3-related), and DNA-PKcs (DNA dependent protein kinase catalytic subunit) (Shiloh Y. Nat Rev Cancer 2003 3:155-168). These kinases initiate signaling cascades upon many types of DNA breaks and activates cell cycle checkpoints and DNA repair pathways (Matsuoka S, et al. Science 2007 316:1160-1166; Paull T T. Annu Rev Biochem 2015 84:711-738). Activated oncogenes induce the stalling and collapse of DNA replication forks, leading to the formation of double-stranded breaks. Advanced tumors often show inactivation of DDR markers, suggesting that silencing of the DDR is an important prerequisite for cancer progression. Once DNA damage is repaired, the cell needs to return to a pre-stress state. The wild-type p53-induced phosphatase 1 (WIP1, also known as PPM1D) appears to be a homeostatic regulator and a master inhibitor in the DDR. It is a type 2C serine/threonine phosphatase that is induced in response to DNA damage in a p53-dependent manner. Previous studies demonstrated that WIP1 dephosphorylates multiple key proteins in the DDR, such as Chk1, Chk2, p53, Mdm2 and H2AX (Emelyanov A, et al. Oncogene 2015 34:4429-4438). Importantly, WIP1 suppresses p53 by multiple mechanisms, including dephosphorylation of p53 kinases (Chk1, Chk2), p53 itself, and Mdm2. Thus, WIP1 facilitates reversal of the DNA damage signaling cascade and reverts the cell to a pre-stress state following completion of DNA repair.

MicroRNAs (miRNAs) are small non-coding RNAs that control gene expression at the post-transcriptional level through translational inhibition and destabilization of their target mRNAs (Bartel D P. Cell 2009 136:215-233). The RNase III enzyme Drosha in the microprocessor complex cleaves pri-miRNAs to pre-miRNAs that contain a characteristic stem-loop structure (Lee Y, et al. EMBO J 2004 23:4051-4060). Pre-miRNAs are then exported to cytoplasm by RanGTP-binding nuclear transporter, Exportin-5. The final step for miRNA maturation is executed by Dicer that cleaves pre-miRNAs into their mature forms (Lin S, et al. Nat Rev Cancer 2015 15:321-333; Chendrimada T P, et al. Nature 2005 436:740-744). The association of miRNA with breast cancer pathogenesis is supported by the studies examining expression of miRNAs in breast cancer cell lines and clinical samples. Expression profiles of miRNAs reflect the lineage and differentiation status of the breast cancer (Blenkiron C, et al. Genome Biol 2007 8:R214). A number of miRNAs are differentially expressed between these molecular tumor subtypes and individual miRNAs are associated with clinicopathological factors.

Consistent with its oncogenic functions, the WIP1 gene in the 17q23 chromosome region is amplified and overexpressed in 11-18% of human breast cancer (Emelyanov A, et al. Oncogene 2015 34:4429-4438; Bulavin D V, et al. Nat Genet 2002 31:210-215). The WIP1-null mice are resistant to spontaneous and oncogene-induced tumors due to enhanced DNA damage and p53 responses (Nannenga B, et al. Mol Carcinog 2006 45:594-604; Bulavin D V, et al. Nat Genet 2004 36:343-350). However, the WIP1 transgene in mouse mammary glands fails to initiate any mammary tumors. While previous studies ruled out the possibility of any protein-coding oncogenes in the WIP1-containing 17q23 amplicon, the disclosed in-depth analysis of human breast cancer genomic DNA revealed an oncogenic miRNA gene, MIR21, in almost all the WIP1 amplicons. Moreover, approximately 81% of the WIP1/MIR21-amplified cancer samples have concurrent HER2 amplification. As disclosed herein, the chromosome 17q23 amplification in the HER2+ breast cancer results in aberrant elevation of WIP1 and miR-21, which not only contributes to breast cancer initiation and progression, but also causes intrinsic resistance to anti-HER2 therapy. Therefore, targeted inhibition of WIP1 and miR-21 could be an effective strategy for the therapy of trastuzumab-resistant HER2+ breast cancer, which has never been explored in the literature.

In this study, a new therapy of trastuzumab-resistant HER2+ breast cancer was developed with the combined use of a small molecular inhibitor against WIP1 (GSK2830371), anti-miR-21 oligonucleotides, and trastuzumab. However, GSK2830371 has poor solubility in water with poor bioavailability in vivo (Gilmartin A G, et al. Nat Chem Biol 2014 10:181-187). Although anti-miR-21 is highly soluble in water, it is relatively unstable in blood. Furthermore, neither GSK2830371 nor anti-miR-21 can efficiently enter cells by itself. To address these challenges, the nanoparticle system described above was used to co-encapsulate GSK2830371 and anti-miR-21 for targeted co-delivery into HER2+ tumor. The disclosed data show that the combined treatment with WIP1 and miR-21 inhibitors co-delivered using the nanoparticle reduces tumor growth by 95% compared to the control groups, confirming that co-inhibition of WIP1 and miR-21 is a promising therapeutic strategy for trastuzumab-resistant HER2+ breast cancer.

Methods

TCGA Analysis. The TOGA breast cancer data were downloaded, which included copy number variation (CNV) at segment level in log-ratio, CNV at gene level estimated by using the GISTIC2 algorithm, RNA-seq for gene expression in base-2 log scale, miRNA mature strand expression data in logarithm (base-2), and patient information about HER2 positive or negative. To study the amp similarity between other genes and WIP1, a focus was placed on only patients with WIP1 amplification. The ratio of patients with amplification on the gene as well was adopted to represent such similarity.

Tissue culture. MCF-7, MDA-MB453, BT474, HMC18, MDA-MB231, MCF10A cell lines were purchased from the American Type Culture Collection. HER18 cells (stably overexpress HER2, parent line MCF-7) were provided (MD Anderson Cancer Center). These cell lines were maintained in Dulbecco's Modified Eagle Medium (DMEM) with 10% FBS at 37° C. in 5% CO₂. MCF10A cells were maintained in DMEM/F12 with 5% Horse Serum EGF (20 ng/ml), Hydrocortisone (0.5 mg/ml), Cholera Toxin (100 ng/ml) Insulin (10 μg/ml). BT474 and HER18 cells were grown and selected in 10 μg/ml Trastuzumab for several weeks and defined as Trastuzumab-resistant cells (HER18-R, BT474-R).

Antibodies and Reagents. Anti-WIP1 antibody (A300-664A), anti-DDX1 (A300-521A), anti-Drosha (A301-886A) and anti-DDX5 (A300-523A) were purchased from Bethyl Laboratories. Anti-HER2 antibody (15212-1-AP) was purchased from proteintech. Anti-p21 (sc-397), anti-GAPDH (sc-20357), anti-Actin (sc-1616), HRP-anti-goat IgG (#2020), HRP-anti-rabbit IgG (#2054) and HRP-anti-mouse IgG (#2055) antibodies were purchased from Santa Cruz. Anti-AKT (4691), anti-phospho-AKT (S473), anti-Chk2 (2662S), anti-phospho-Chk2 (2661S) anti-cleaved caspase3 (9661S) were purchased from Cell Signaling. Trastuzumab (Herceptin, Genentech), GSK-2830371 (Active Biochem) were used to target HER2 and WIP1 in vitro and in vivo. Antisense miR-21 miRZip (System Bioscience) were stably expressed to inhibit miR-21 function in the cells. To evaluate and measure miR-21 in the cells, pmirGLO Dual Luciferase miR-21 vector was used in the cells. mirVana miR-21 inhibitor (Ambion, Life Technology) was used for in vivo miR-21 inhibition. PLGA (lactide:glycolide=75:25, Mw: 4,000-15,000), PF127, and organic solvents were purchased from Sigma (St. Louis, Mo., USA). Agarose, ethidium bromide and loading buffer were purchased from Thermo Fisher Scientific (Grand Island, N.Y., USA). DPPC was purchased Anatrace (Maumee, Ohio, USA). Chitosan oligosaccharide of pharmaceutical grade (Mw: 1.2 kD, 95% deacetylation) was purchased from Zhejiang Golden Schell Biochemical Co. Ltd (Zhejiang, China). Methyl aminomethanimidothioate hydroiodide was purchased from Santa Cruz Biotechnology (Dallas, Tex., USA).

Generation of Doxycycline-inducible knocked-down cell lines. Lentiviral pGIPZ vector expressing non-specific silencing shRNA control, WIP1 and DDX5 shRNAs were obtained from the MD Anderson shRNA and ORFome Core Facility (originally from Open Biosystem) and sub-cloned to pTRIPZ vector according to the manufacturer's instructions. To generate Dox-inducible anti-sense miR-21 miRZip expressing cell lines, anti-microRNA expression cassette was sub-cloned to pLKO-Tet-On vector. Cells were infected with lentiviruses in the presence of polybrene (8 μg/ml). To establish stable knockdown cell line, lentiviral shRNA-transduced cells were selected with puromycin (2 μg/ml) 48 h post-infection and individual colonies were propagated and validated for expression by Western blotting (protein) and qRT-PCR (mRNA).

Genomic DNA isolation and copy number validation. Total genomic DNA was extracted from human cell lines using DNeasy Blood & Tissue Kit (Qiagen) according to the manufacturer's purification instructions. The copy number validation for HER2, WIP1, and MIR21 were determined by quantitative PCR assays using iTaq Universal SYBR Green Supermix (Bio-Rad) on an Applied Biosystems 7900HT Sequence Detection System.

Immunoblotting. Immunoblotting was performed as described previously (Liu Y, et al. Nature 2015 520:697-701). Briefly, total cell lysates were solubilized in lysis buffer (50 mM Tris, pH 7.5, 150 mM NaCl, 1 mM EDTA, 0.5% NP-40, 0.5% Triton X-100, 1 mM phenylmethylsulfonyl fluoride, 1 mM sodium fluoride, 5 mM sodium vanadate, 1 μg each of aprotinin, leupeptin, and pepstatin per ml). Proteins were resolved by SDS-PAGE gels and then proteins were transferred to PVDF membranes (Bio-Rad). The membranes were blocked with 5% nonfat milk for 1 h at room temperature prior to incubation with indicated primary antibodies. Subsequently membranes were washed and incubated for 1 h at room temperature with peroxidase-conjugated secondary antibodies (Santa Cruz Biotechnology). Following several washes, chemiluminescent images of immunodetected bands on the membranes were recorded on X-ray films using the enhanced chemiluminescence (ECL) system (Perkinelmer) according to the manufacturer's instructions.

Cell viability assay. Equal numbers of cells were plated in 12 well plates in triplicate. After incubation with indicated reagents for 72 hours, cell viability was quantified using Vi-Cell cell viability analyzer (Beckman Coulter).

Soft agar colony formation assay. 2000 cells per well mixed in a 0.35% agarose/complete media suspension were seeded onto 0.7% agarose/complete media bottom layer. Three weeks later, 100 μl per well of p-iodonitrotetrazolium violet (1 mg/ml, Sigma) was added for 16 h before photographed.

Mammosphere culture. Mammary tumor cells were plated onto ultralow attachment plates (Corning) at a density of 20,000 viable cells/mL (to obtain primary mammospheres) in a serum-free DMEM-F12 (Invitrogen) supplemented with 5 μg/mL insulin, 20 ng/mL epidermal growth factor and 20 ng/mL basic fibroblast growth factor (Sigma), and 0.4% bovine serum albumin (Sigma). After 10 days, number and size of mammospheres were estimated.

Immunoprecipitation. Cells were lysed on ice for 30 min in IP buffer (1% NP-40, 50 mM Tris-HCl, 500 mM NaCl, 5 mM EDTA) containing protease inhibitor cocktail. Cell lysates (700 μg) were incubated overnight with 3 μg of antibodies or normal IgG at 4° C. with rotary agitation. Protein A-sepharose beads were added to the lysates and incubated for additional 4 h. Beads will be washed three times with IP buffer and boiled for 10 min in 3% SDS sample buffer. Total cell lysates and immunoprecipitates were separated by SDS-PAGE and analyzed by Western blotting.

RNA immunoprecipitation (RIP) assay. Cell were crosslinked for 20 min with 1% formaldehyde, and cell pellets were resuspended in buffer B (1% SDS, 10 mM EDTA, 50 mM Tris-HCl (pH 8.1), 1× protease inhibitor, 50 U/ml RNase inhibitor). Incubated 10 min in ice, the pellets were disrupted by sonication, and the lysates were subjected to immunoprecipitation with control IgG or anti-DDX1 or anti-Drosha antibody, followed by stringent washing, elution, and reversal of crosslinking. The RNA was resuspended in 20 μl of TE buffer (10 mM Tris-HCl, pH 7.5, 1 mM EDTA, 50 U/ml RNase inhibitor) and incubated with DNase I for 30 min at 37° C. to remove any remaining DNA. After extraction with phenol:chloroform:isoamyl alcohol (25:24:1), RNA was precipitated with ethanol and dissolved in 20 μl of DEPC-treated water. RNA (5 μl) was used for the cDNA synthesis reaction. Quantitative PCRs were then performed on real-time PCR machine.

RNA isolation, qRT-PCR, and miRNA PCR array. Total RNA was isolated using TRIzol reagent (Life Technologies) and then reverse-transcribed using iScript cDNA Synthesis Kit (Bio-Rad). The resulting cDNA was used for qPCR using iTaq Universal SYBR Green Supermix (Bio-Rad) with gene-specific primers and the results were normalized to β-actin control. To analyze miRNAs, total RNA was isolated using Trizol reagent according to the manufacturer's instructions (Life Technologies) and was then reverse transcribed with a Universal cDNA Synthesis Kit II (Exiqon). cDNA was used for qPCR using SYBR Green master mix (Exiqon) on ABI4900 real-time PCR cycler. miRNA LNA PCR primer sets (Exiqon) and gene specific primers were used for detecting miRNA and mRNA levels and data was normalized to internal control, U6 (miRNA) or GAPDH (mRNA). RT-PCR primers are shown in Table 1.

TABLE 1
Primer sequences for real-time PCR.

| | |
|---|---|
| pri-miR-21-forward | TTTTGTTTTGCTTGGGAGGA (SEQ ID NO: 6) |
| pri-miR-21-reverse | AGCAGACAGTCAGGCAGGAT (SEQ ID NO: 7) |
| pre-miR-21-forward | ATGTTGACTGTTGAATCTCATGG (SEQ ID NO: 8) |
| pre-miR-21-reverse | TGTCAGACAGCCCATCGAC (SEQ ID NO: 9) |
| pri-miR-200a-forward | GATGCAAGGGTCAGAAGGGC (SEQ ID NO: 10) |
| pri-miR-200a-reverse | GAGCCATCTGGCCCGGACG (SEQ ID NO: 11) |
| pri-miR-16-forward | GCAATTACAGTATTTTAAGAGATGAT (SEQ ID NO: 12) |
| pri-miR-16-reverse | CATACTCTACAGTTGTGTTTTAATGT (SEQ ID NO: 13) |
| DDX1-forward | TTGATGGGAAAGTTACCTACGG (SEQ ID NO: 14) |
| DDX1-reverese | CAAGATGCAGGAAAGATGTCTG (SEQ ID NO: 15) |
| DDX5-forward | CACAGCATACACTTTCTTTACACC (SEQ ID NO: 16) |
| DDX5-reverse | TGGAACGACCTGAACCTCTG (SEQ ID NO: 17) |
| GAPDH-forward | AGCCACATCGCTCAGACAC (SEQ ID NO: 18) |
| GAPDH-reverse | GCCCAATACGACCAAATCC (SEQ ID NO: 19) |
| WIP1-forward | AGAAGCAGAAGGGTTTCACCT (SEQ ID NO: 20) |
| WIP1-reverse | CATTCCGCCAGTTTCTTCCAC (SEQ ID NO: 21) |
| HER2-forward | TGCAGGGAAACCTGGAACTC (SEQ ID NO: 22) |
| HER2-reverse | ACAGGGGTGGTATTGTTCAGC (SEQ ID NO: 23) |

miR-21 in situ hybridization. Breast cancer tissue array (BR10010) was purchased from Biomax.us, containing 50 cases of breast carcinoma. As described previously (Huo L, et al. Mod Pathol 2016 29:330-346), the tissue slides were first digested with 15 μg/ml proteinase K for 10 min at room temperature, and then hybridized with the double-DIG-labeled mercury LNA microRNA probe (Exiqon) for 2 h at 50° C. on Ventana Discovery Ultra (Ventana Medical Systems). The digoxigenins were then detected with a polyclonal anti-DIG antibody and alkaline phosphatase-conjugated second antibody using NBT-BCIP as the substrate. Raw images were captured with the same exposure and gain settings from all slides and saved as TIF files, and were analyzed using intensity measurement tools of Image-Pro Plus software.

Synthesis of nanoparticles. The nanoparticles were synthesized using a double emulsion method with a slight modification (Wang H, et al. Adv Mater 2016 28:347-355). First, Chitosan was modified with guanidine group according to the literature (Bottcher T, et al. J Am Chem Soc 2013 135:2927-2930) to form chitosan-guanidine (CG). To capture carbon dioxide (CO₂), CG aqueous solution was bubbled with CO₂ for 1 h to form CG-CO₂. PLGA (20 mg) and DPPC (10 mg) were dissolved in 2 mL dichloromethane (DCM) and 50 μL GSK2830371 in tetrahydrofuran (THF) solution (40 mg/mL) was then added, the above mixture together with 400 μL of DI water containing 500 μg/mL anti-miR21 oligonucleotide and 500 μg/mL CG-CO₂ were transferred into a 50 mL centrifuge tube. Then the immiscible solutions were emulsified by sonication for 1 min using a Bransan 450 sonifier. Next, this first emulsion and 8 mL of chitosan-PF127 solution (in DI water) were emulsified by sonication for 1 min. After rotary evaporation to remove the organic solvent, the nanoparticles were collected by centrifugation at 10000 g for 10 min at room temperature and washed twice with DI water.

Characterization of nanoparticles. The nanoparticle was characterized using both transmission electron microscopy (TEM) and dynamic light scattering (DLS). First, nanoparticles were soaking in soaking in Phosphate Buffer (pH 7.4, pH 6.0) or Acetate Buffer (pH 5.0), respectively, for 6 h. For TEM study, the nanoparticles were negatively stained with uranyl acetate solution (2%, w/w) and examined using an FEI (Moorestown, N.J., USA) Tecnai G2 Spirit transmission electron microscope. The nanoparticle size was determined using a Brookhaven (Holtsville, N.Y., USA) 90 Plus/BI-MAS dynamic light scattering instrument.

Electrophoretic gel assay. Free anti-miR21 oligonucleotide and in-MW@NP (in PBS or serum) were mixed with loading buffer, and then loaded into 2% wt agarose gel with 0.5 mg/mL ethidium bromide. Electrophoresis was conducted in 1×TEA buffer at 80 V for 10 min. The result gels were analyzed using a UV illuminator (FluorChem™ E System, CA, USA) to show its location of anti-miR21 oligonucleotide.

Cell uptake and intracellular distribution of nanoparticles. Dex-Rho was encapsulated inside the nanoparticles using the same method described above. To study cellular uptake and subcellular localization of nanoparticles, HER8R cells were treated with Dex-Rho@NP or free Dex-Rho for 1-6 h at 37° C. The cells were further treated with medium containing 90 nM LysoTracker Green and 50 nm DAPI. Then, the cells were further examination using an Olympus FluoView™ FV1000 confocal microscope.

Breast tumor xenograft mouse model. Female NOD/SCID mice (6-8 weeks old) were purchased from Jackson Laboratories. All studies were approved and supervised by the Institutional Animal Care and Use Committee at the MD Anderson Cancer Center. For the breast cancer orthotopic xenograft model, nude mice were injected with 6×10⁶ HER18 human breast cancer cells in the mammary fat pad as described previously (McKenzie T, et al. Surgery 2004 136:437-442; Warburton C, et al. Clin Cancer Res 2004 10:2512-2524). The nude mice were supplementary with weekly subcutaneous estradiol cypionate injections (3 mg/kg/week), starting 1 week prior to injection of tumor cells. After initial establishment of tumor (50 mm³), mice were randomly grouped and treated with or without 1 μg/ml Dox in drinking water for 4 weeks. The Dox water was changed every other day. The administration of trastuzumab (5 mg/kg) was performed biweekly for 4 consecutive weeks by intraperitoneal injection.

For xenograft tumor studies using nanoparticles, mice bearing trastuzumab-resistant HER18 tumors were randomized to four groups (n=8) and received the following treatments: 1) control nanoparticles; 2) WIP1 inhibitor nanoparticles; 3) miR-21 inhibitor nanoparticles; and 4) WIP1 inhibitor+miR-21 inhibitor nanoparticles. Nanoparticles were administered twice weekly by intraperitoneal injection, and tumor size and body weight changes were monitored biweekly. Tumor size was measured using a caliper, and tumor volume was calculated using the standard formula: 0.5×L×W², where L is the longest diameter and W is the shortest diameter. Mice were euthanized when they met the institutional euthanasia criteria for tumor size and overall health condition. Tumors were removed, photographed and weighed. The freshly dissected tumor tissues were fixed in 10% buffered formalin overnight, transferred to 70% ethanol, embedded in paraffin, sectioned and stained with hematoxylin and eosin and indicated antibodies.

Immunohistochemistry. The tumors were fixed in 10% neutral buffered formalin and embedded in paraffin and 5-μm tissue sections were serially cut and mounted on slides. The sections were de-paraffinized in xylene, re-hydrated, and boiled for 10 min in antigen retrieval buffer. After retrieval, the sections were washed with distilled water and endogenous peroxidase activity was blocked using 3% H₂O₂ in TBS for 15 min and then blocked with blocking solution (1% bovine serum albumin, 10% normal serum in 1×TBS). Samples were incubated with primary antibodies overnight at 4° C., washed three times with TBST buffer, and then incubated with biotinylated goat anti-rabbit or anti-mouse IgG (GR608H, BioCare Medical). A streptavidin-biotin peroxidase detection system with 3,3′-diaminobenzidine as substrate was used according to the manufacturer's instructions (DAB Peroxidase Substrate Kit, Vector Laboratory). Sections were counterstained with haematoxylin. For immunocytochemistry, cells on chamber slide were fixed in 3.7% paraformaldehyde for 10 min, and permeabilzed by 0.2% Triton X-100 for 5 min and then blocked with blocking solution. After incubation with primary antibodies, cells were washed with PBS three times and incubated with Alexa fluor 488 or 594 conjugated antibodies (Life Technologies) and counter stained with Hoechst 33258. After mounting, signals were observed under microscope. Breast cancer tissue array (BR10010) was purchased from Biomax.us.

Results

Co-Amplification of WIP1 and MIR21 in the 17q23 Amplicon of HER2+ Breast Cancers

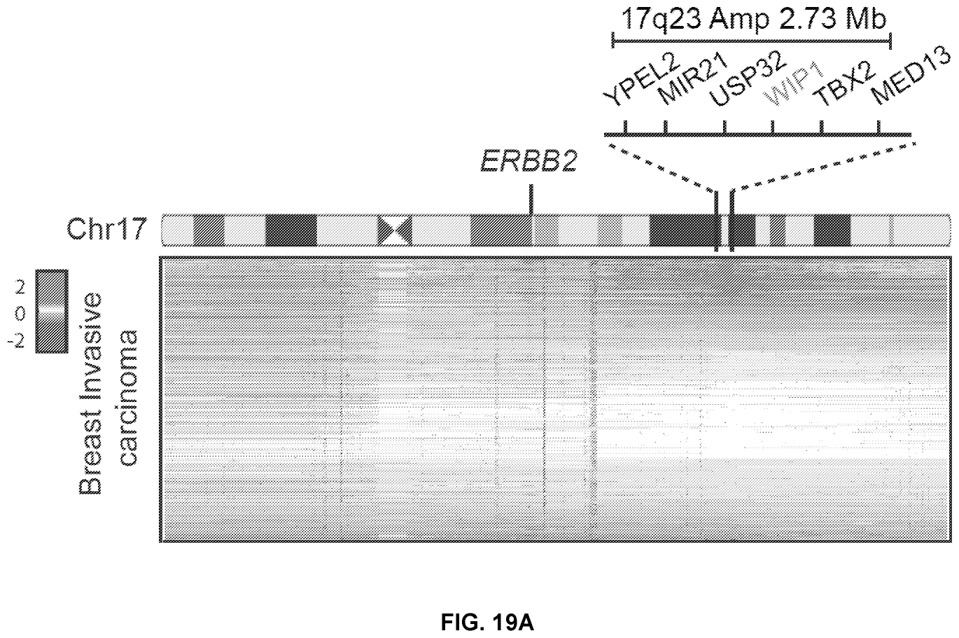

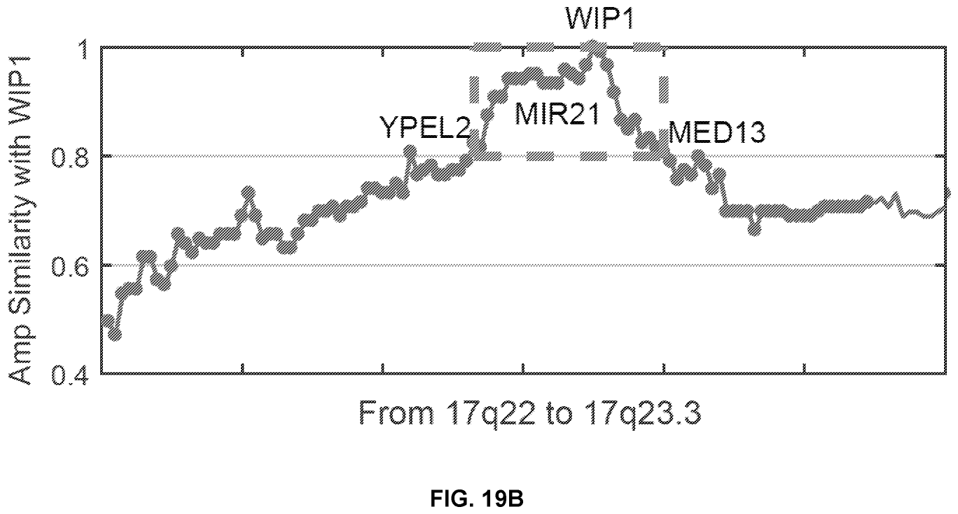

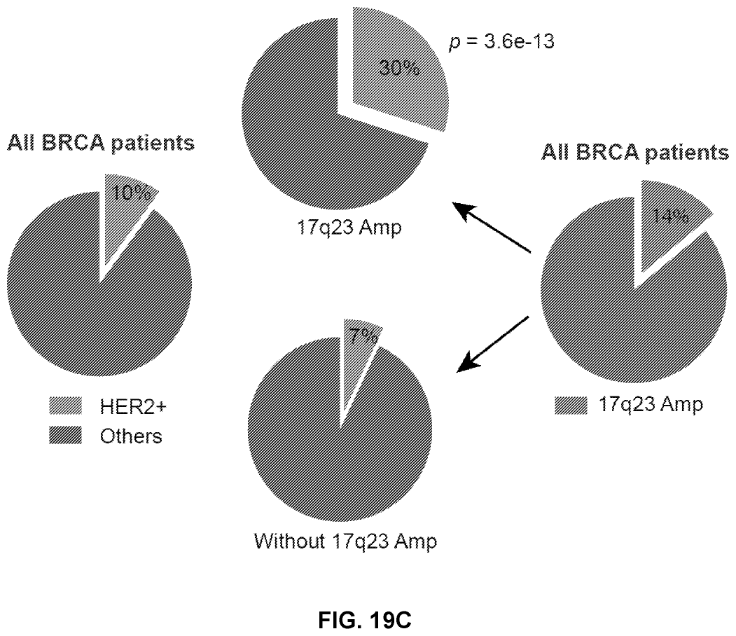

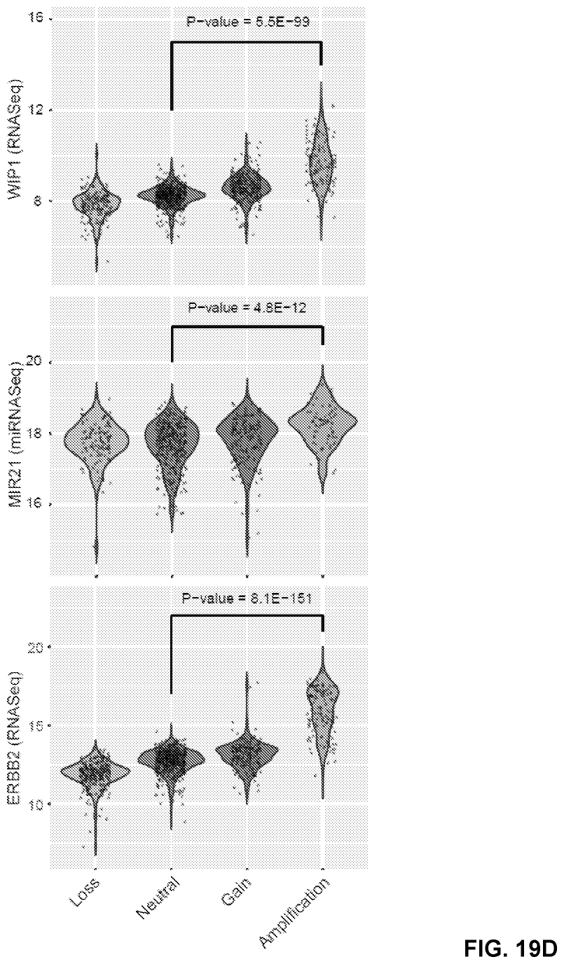

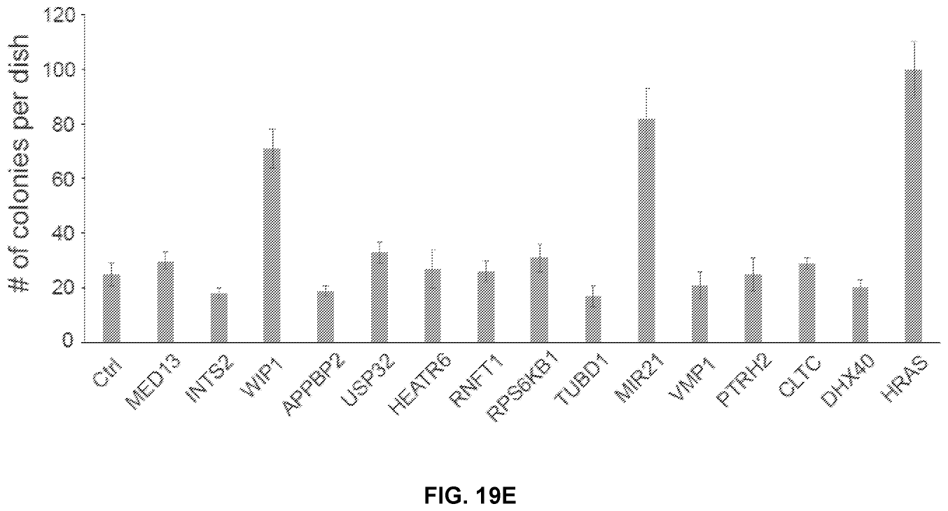

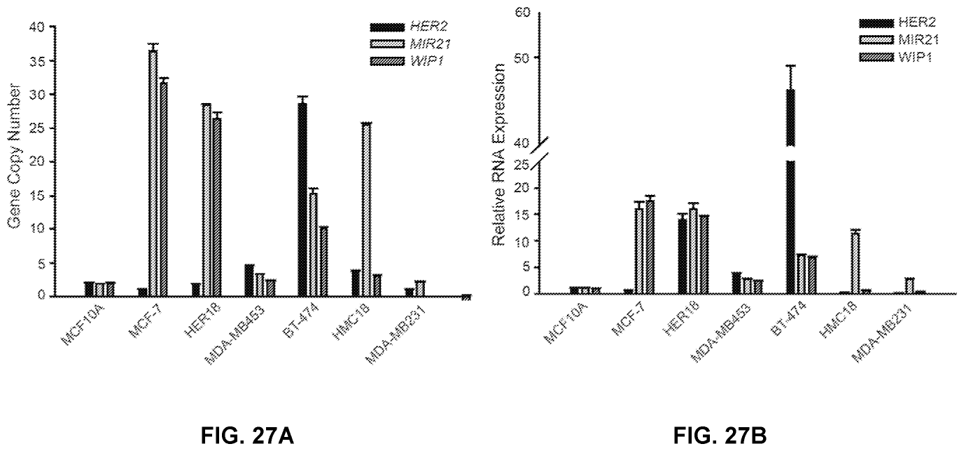

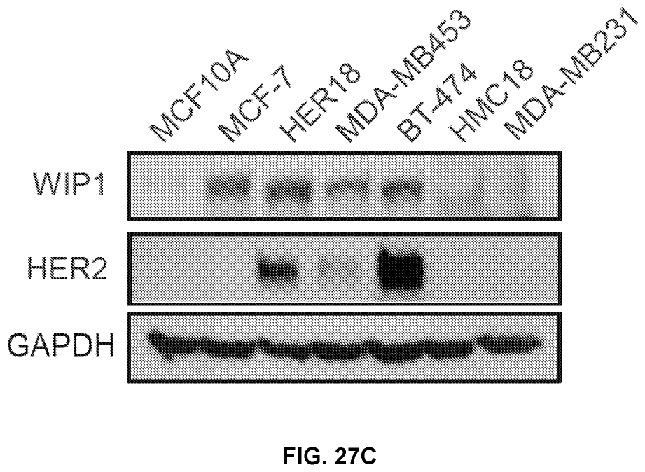

The W/P1-containing 17q23 region is amplified in a subset (˜11%) of human breast tumors (Li J, et al. Nat Genet 2002 31:133-134). Extensive analyses of breast cancer genomics revealed that the 17q23 amplicon can span up to over 10 Mb, including a number of protein-coding and non-coding genes. But previous studies identified WIP1 as the only oncogene in the amplicon due to incomplete breast cancer genomic databases and lack of noncoding RNA information. To search for other potential genes that potentially promote breast cancer progression, the 17q23 amplicon was analyzed based on the data downloaded from the TOGA (The Cancer Genomics Atlas) breast cancer databases (Cancer Genome Atlas N. Nature 2012 490:61-70; Ciriello G, et al. Cell 2015 163:506-519). The results revealed that the amplicon region for some breast tumors focused an approximately 2.73 Mb region (FIG. 19A), where 21 protein-coding genes (MED13, INTS2, BRIP1, NACA2, TBX4, C17ORF82, TBX2, BCAS3, APPBP2, C170RF64, USP32, CA4, HEATR6, RNFT1, RPS6KB1, TUBD1, VMP1, PTRH2, CLTC, DHX40, YPEL2) and 3 miRNA genes (MIR4737, MIR21, MIR4729) showed highly similar amplification profiles to WIP1 (FIG. 19B). One interesting thing is that transgenic mice overexpressing WIP1 in mammary glands showed no abnormal overt phenotype on development, lactation or involution of mouse mammary glands, and did not develop spontaneous mammary tumors (Demidov O N, et al. Oncogene 2007 26:2502-2506). However, mammary tumor incidence of the WIP1-transgenic mice was accelerated when these animals were crossed with mammary tumor susceptible ErbB2 (the mouse homolog of HER2) transgenic mice, suggesting that WIP1 plays an important role in HER2-initiated breast cancer. As a matter of fact, the WIP1 amplification was enriched in the HER2+ breast cancer (FIG. 19C). Even though only 10% of all types of breast cancer in the dataset were identified as HER2+, the HER2+ subtype existed in 30% (3-fold more, p=3.6e-13) of the WIP1-amplified breast cancers, compared to 7% of breast cancers without WIP1 amplification (FIG. 19C). The RNA-seq or miRNA-seq analysis also revealed that 14 out of 24 protein-coding and non-coding genes in the amplicon were significantly (p<0.001) overexpressed with the gene amplification than in those neutral cases, for instance, WIP1 as well as MIR21, a well characterized oncomiR, which both were similar to the aberration of ERBB2 (FIG. 19D). These observations suggest that gene amplification results in higher expression levels of WIP1 and miR-21 in the HER2+ breast cancer (FIG. 27). Furthermore, the transformation ability of the 13 protein-coding genes and MIR21 that are overexpressed in the 17q23-amplified breast cancers was assessed (FIG. 19E). Soft-agar colony formation assays were performed by transducing primary mammary epithelial cells from MMTV-ErbB2 transgenic mice with lentivirus expressing each individual gene. The results demonstrated that both WIP1 and MIR21, but not any other genes in the amplicon, induced in vitro transformation, indicating their functional roles in HER2+ breast cancers.

Suppression of miR-21 and WIP1 Inhibits Proliferation and Tumorigenic Potential of HER2+ Breast Cancer Cells

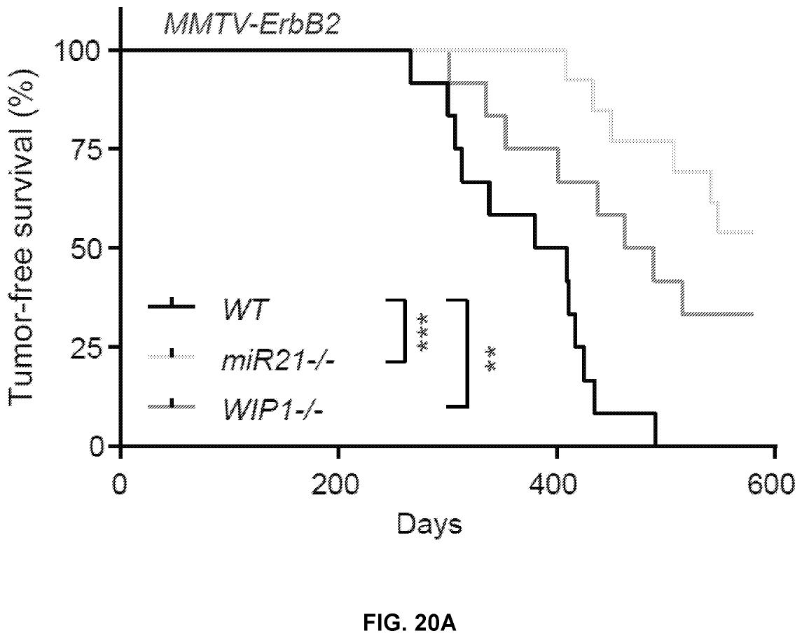

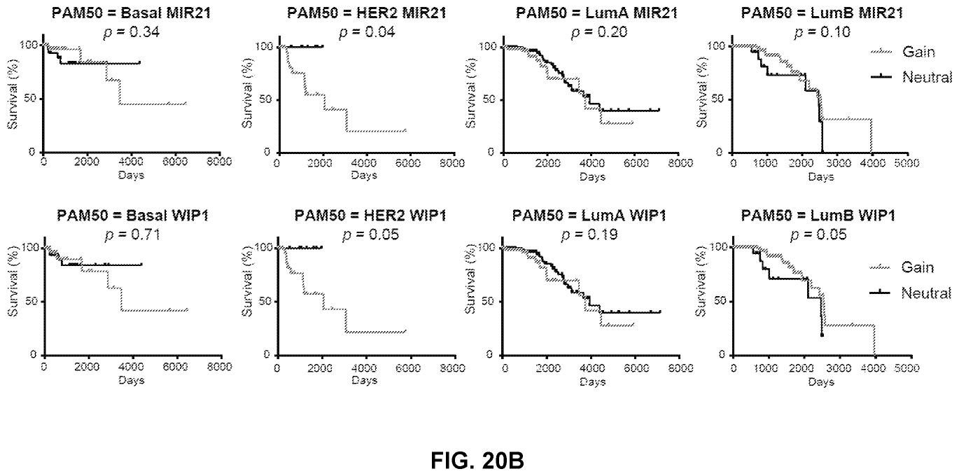

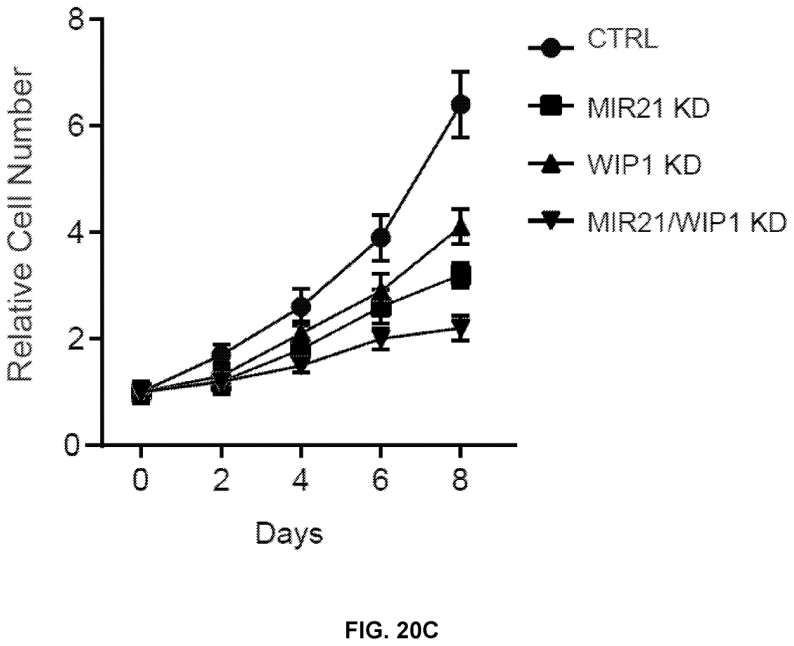

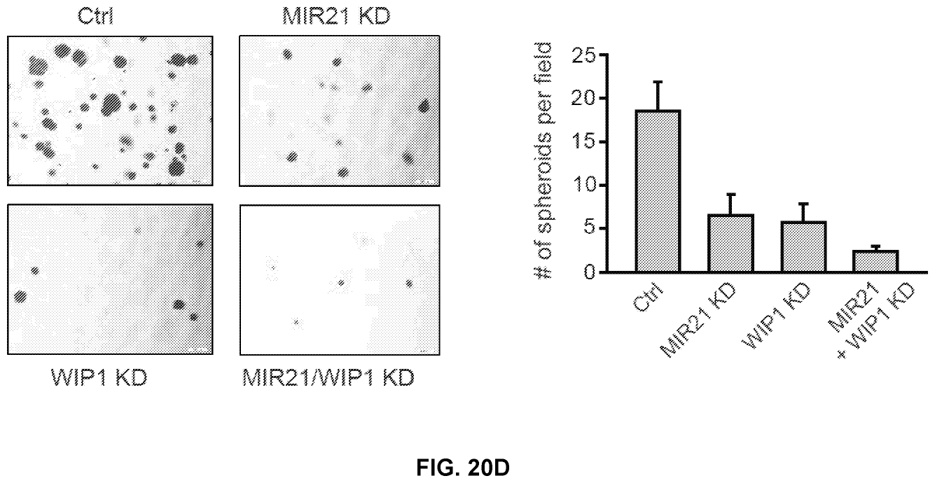

It has been known that both WIP1^{-/-} and MIR21^{-/-} mice have normal mammary gland development and functions (Bulavin D V, et al. Nat Genet 2004 36:343-350; Li J, et al. Nat Genet 2002 31:133-134; Ma X, et al. Proc Natl Acad Sci USA 2011 108:10144-10149), suggesting that WIP1 and miR-21 are dispensable for mammary development and normal physiological functions of mammary epithelial cells. To study their potential oncogenic roles, mammary tumorigenesis of MMTV-ErbB2 transgenic mice were examined in the contexts of WIP1 or MIR21 knockout (FIG. 20A). All the females in the experimental groups were kept virgin during the 20-month observation period. All the MMTV-ErbB2 transgenic female mice (12 out of 12) died of mammary tumors before the end of observation period. The absence of WIP1 impaired mammary gland tumor progression induced by the ErbB2 transgene, consistent with previous studies (Nannenga B, et al. Mol Carcinog 2006 45:594-604; Bulavin D V, et al. Nat Genet 2004 36:343-350). Similar to the WIP1 knockout, depletion of miR-21 rendered the MMTV-ErbB2 mice considerably more resistant to tumor formation. Only 6 out of 13 MIR21^{-/-};MMTV-ErbB2 females developed mammary tumors (p<0.001). Overall, WIP1^{-/-};MMTV-ErbB2 and MIR21^{-/-};MMTV-ErbB2 females showed a statistically significant increase in lifespan in comparison with their control MMTV-ErbB2 littermates. The results suggest that both MIR21 and WIP1 deficiencies overcome the tumor-promoting activity of ErbB2. Consistent with the results from mouse models, the analysis of breast cancer genomics using TCGA databases showed that copy number gains of WIP1 or MIR21 are significantly correlated with poor clinical outcomes in patients with HER2+ breast cancer, but not in patients with luminal A, luminal B or basal-like breast cancer (FIG. 20B). The results supported the tumor-promoting roles of WIP1 and miR-21 in breast cancer. Next assessed was whether inhibiting WIP1 or miR-21 impact the proliferation and mammosphere formation of the tumor cells (H605), which were isolated and established from mouse MMTV-ErbB2 mammary tumors (Zhang X, et al. Cancer Res 2010 70:7176-7186). Either WIP1 or miR-21 knockdown markedly reduced the tumor cell growth rate, whereas knockdown of both had a more profound inhibition of cell proliferation (FIG. 20C). Floating mammosphere formation assay was used to assess the stemness and tumorigenic potential of tumor cells. Depletion of WIP1 or miR-21 significantly diminished the number and size of mammospheres formed by H605 cells, and knockdown of both WIP1 and miR-21 further inhibited the mammosphere formation. These results validated the oncogenic potential of both WIP1 and MIR21 genes (FIG. 20D).

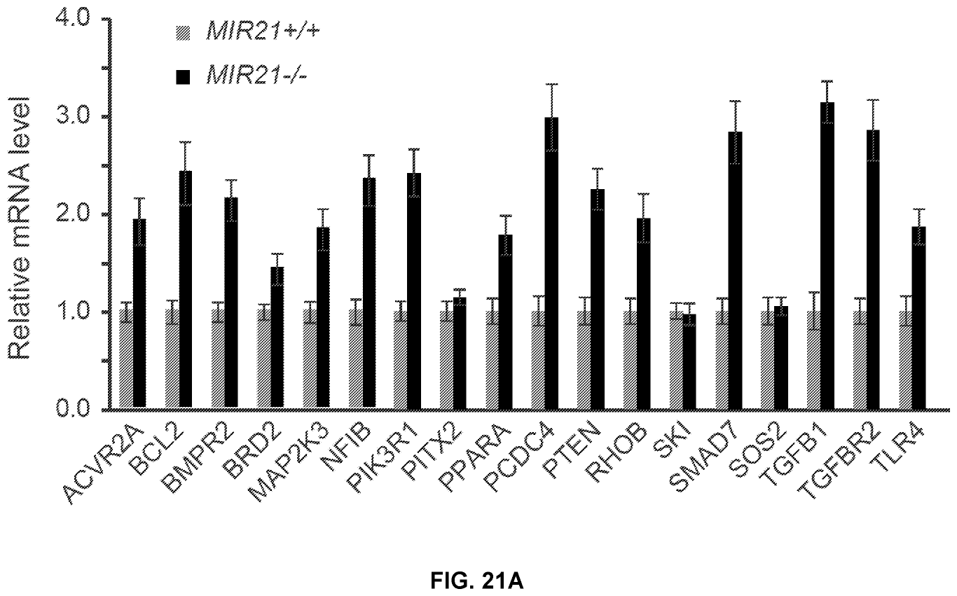

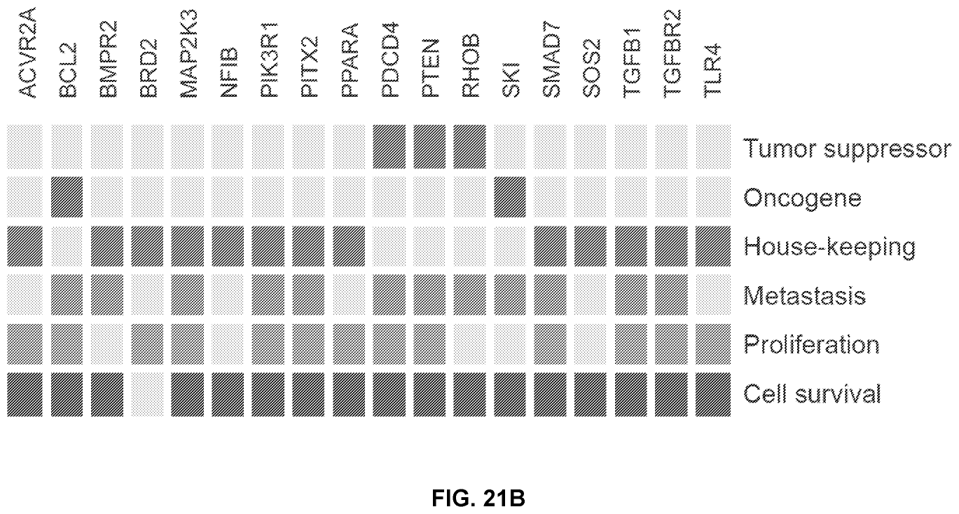

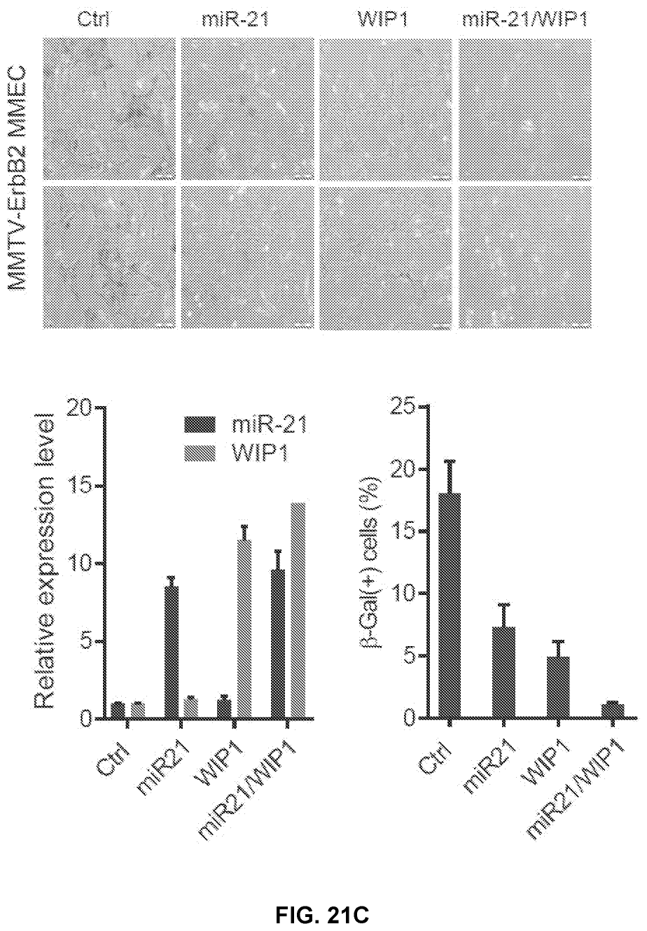

Oncogene-induced senescence (01S) in mammary glands is a physiologically protective mechanism against breast cancer (Milanese T R, et al. J Natl Cancer Inst 2006 98:1600-1607). Accumulating evidence supports the important role of the ATM-p53 signaling pathway in OIS (Bartkova J, et al. Nature 2005 434:864-870). However, recent studies pointed out that TGF-β signaling pathway is responsible for the ATM-independent OIS in mammary glands (Cipriano R, et al. Proc Natl Acad Sci USA 2011 108:8668-8673). While WIP1 is a master inhibitor for the ATM-p53 signaling, miR-21 may suppress TGF-β signaling via negative regulation of its targets in the pathway. To identify physiological relevant miR-21 targets, several algorithms (TargetScan, PITA, microT and PicTar) were first used to predict miRNA-21 targets and binding sites. Transcripts of 18 genes were identified as potential miR-21 targets in mammary cells, which modulate crucial tumor cell activities (survival and proliferation) (FIG. 21A). Gene expression analyses were performed to validated these target genes in miR-21+/+ and miR-21−/− mouse mammary glands. Mouse mammary glands were harvested from 8-wk littermate females and the purified mRNAs were subjected to reverse transcription and quantitative PCR analyses (FIG. 21B). Interestingly, a number of miR-21 targets are associated with the TGF-β signaling pathway, including TGFB1, TGFBR2, ACVR2A, SMAD7, PCDC4, and BMPR2. No overlaps were identified between WIP1 and miR-21 targets, suggesting that these two oncogenes play non-redundant roles in mammary tumorigenesis. Mouse mammary epithelial cells (MMECs) isolated from MMTV-ErbB2 transgenic mouse were passaged and examined for oncogene-induced senescence. The MMECs, after three passages, exhibited an increase in cell size, cell spreading, vacuolization, multinucleated morphology and positive staining for the presence of senescence-associated β-galactosidase (SA-13-Gal) activity, characteristic of cell senescence (FIG. 21C). In line with the results in regard to the transforming activity of miR-21 and WIP1, ectopic expression of WIP1 or miR-21 alone partly rescued MMECs from senescence, indicated by reduced number of cells positive for SA-β-Gal. Co-expression of WIP1 and miR-21 had a profound inhibition on the OIS of MMECs overexpressing ErbB2, suggesting that amplification of WIP1 and miR-21 may allow MMECs to evade from OIS and promotes their oncogenic transformation.

DDX5 Gene is Co-Amplified with MIR21 and DDX5 Facilitates Maturation of Pri-miR-21

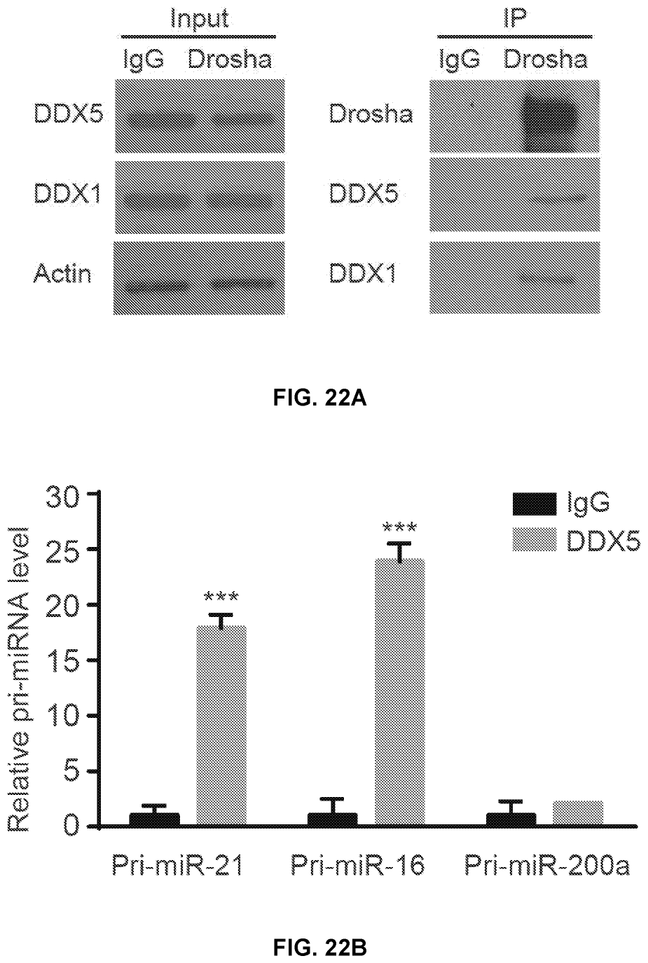

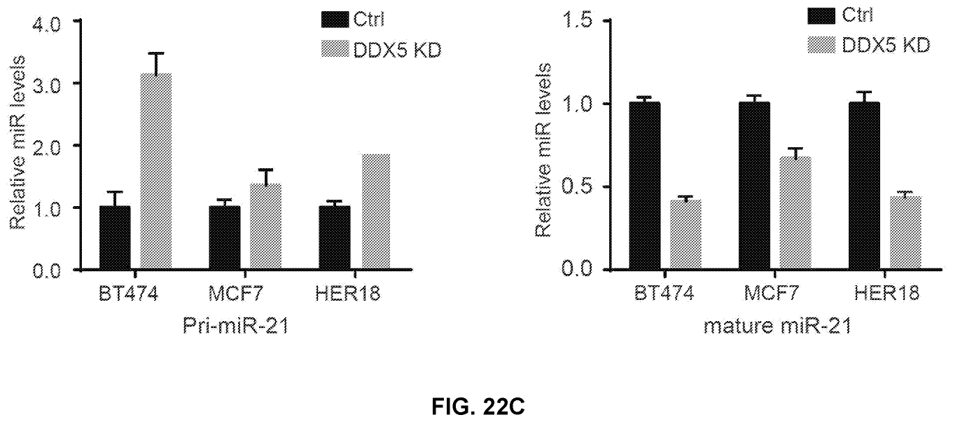

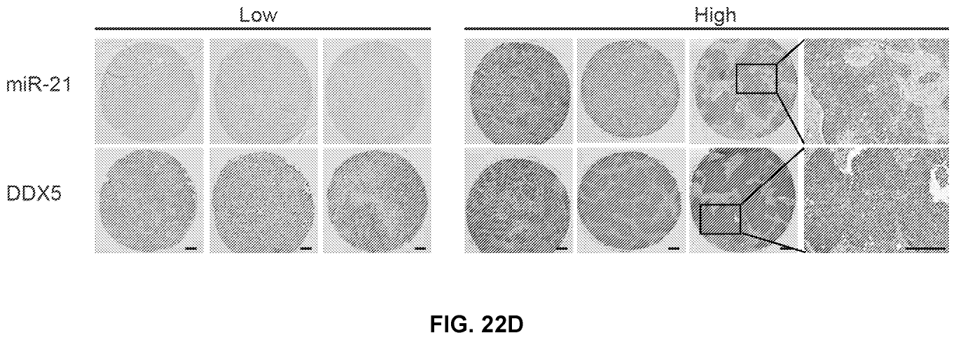

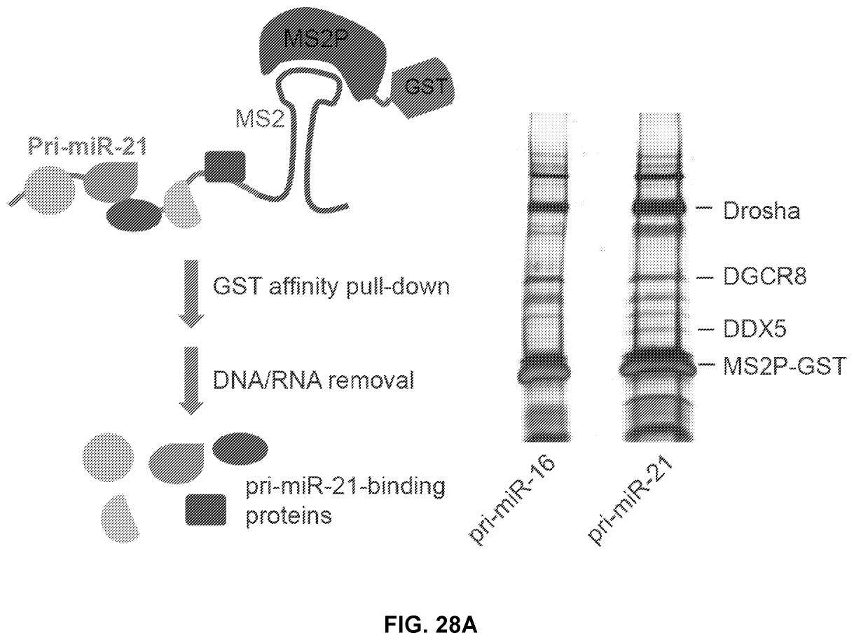

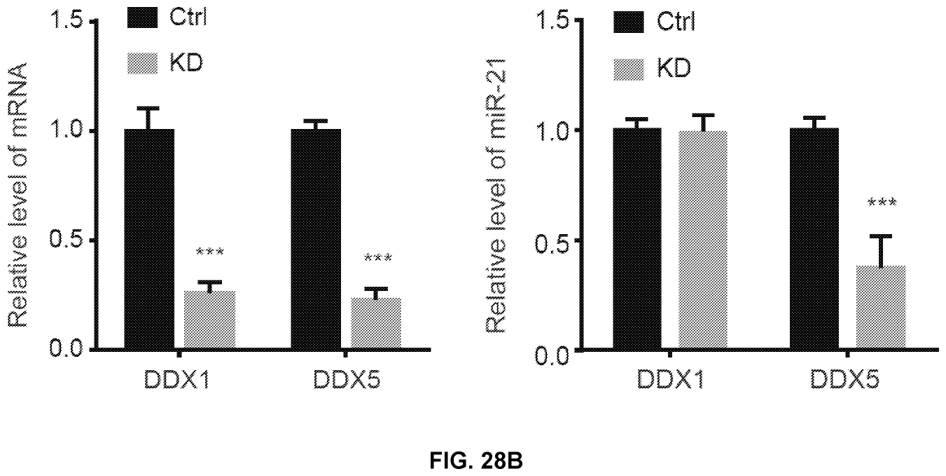

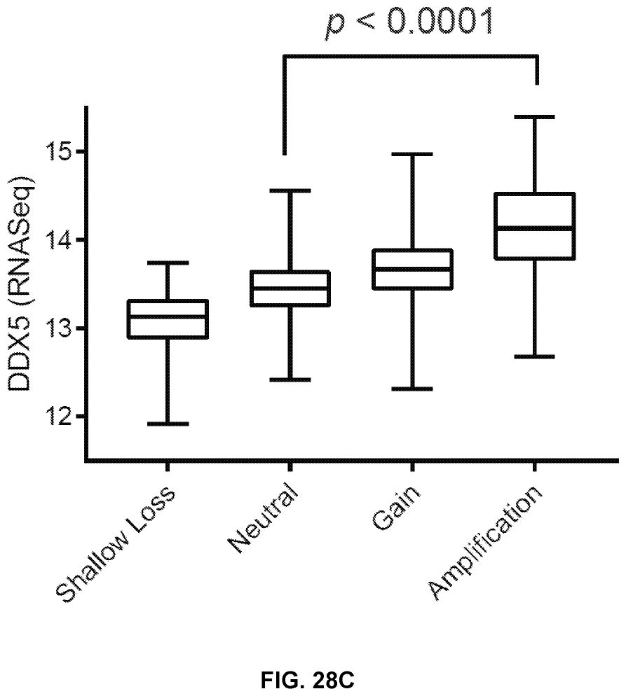

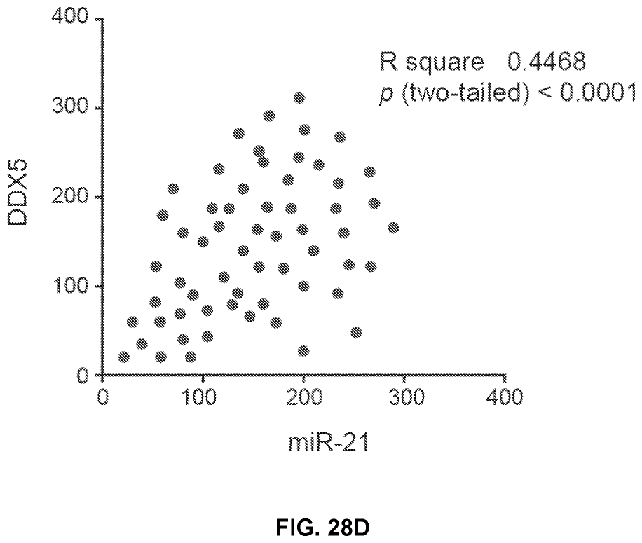

While no miRNA-sequence specificity exists for Drosha and DGCR8, two core components in the microprocessor, emerging evidence has shown that regulatory RNA-binding proteins in the Drosha complexes may recruit specific primary miRNAs (pri-miRs) for processing (Wan G, et al. Antioxid Redox Signal 2014 20:655-677). A MS2-TRAP (MS2-tagged RNA affinity purification) assay was performed to identify a pri-miR-21-specific regulatory component in the Drosha microprocessor (Yoon J H, et al. Methods 2012 58:81-87). The assay is based on the addition of a specific MS2 RNA hairpin loop sequence (from bacteriophage MS2) to pri-miR-21, followed by co-expression of the MS2-tagged RNA together with GST-tagged MS2P that specifically binds the MS2 RNA sequence. In addition to those predicted proteins (Drosha and DGCR8) in the microprocessor complex, DEAD-Box helicase 5 (DDX5) was identified in the pri-miR-21-protein complex (FIG. 28A). Interaction of DDX5 with Drosha was further confirmed in the immunoprecipitation and western blotting assay (FIG. 22A). A regulatory component in the microprocessor identified in a previous study, DDX1, was used as a positive control for the interaction with Drosha (Han C, et al. Cell Rep 2014 8:1447-1460). RNA immunoprecipitation (RIP) assays further verified specific interaction between endogenous DDX5 and pri-miR-21 (FIG. 22B). Pri-miR-16 was also a DDX5-interacting RNA as previously reported. As a DDX5-independent negative control, pri-miR-200a had no interaction with DDX5 (Han C, et al. Cell Rep 2014 8:1447-1460). Knockdown of DDX1 inhibited the processing of pri-miR-21 and resulted in accumulation of unprocessed pri-miR-21 and decreased levels of mature forms of miR-21 in three breast cancer cell lines harboring 17q23 amplicon (FIGS. 22C and 28B). Genomic analysis of HER2+ breast cancer revealed that DDX5 is adjacent to the WIP1-MIR21 amplicon and is co-amplified with 67% of HER2+ breast cancer with MIR21 amplification. Consistent with the results from the analysis of miR-21 and DDX5 mRNA expression levels using TCGA breast cancer databases (FIG. 28C), DDX5 protein levels are positively correlated with miR-21 levels, determined by immunohistochemistry staining of HER2+ breast tumor tissue microarray (FIGS. 22D and 28D). Collectively, DDX5 is potentially an important player that promotes the oncogenic function of the 17q23 amplicon via facilitating miR-21 expression.

Inhibition of WIP1 Kills Breast Cancer Cells Carrying 17q23 Amplicon Only in the Presence of Wildtype p53

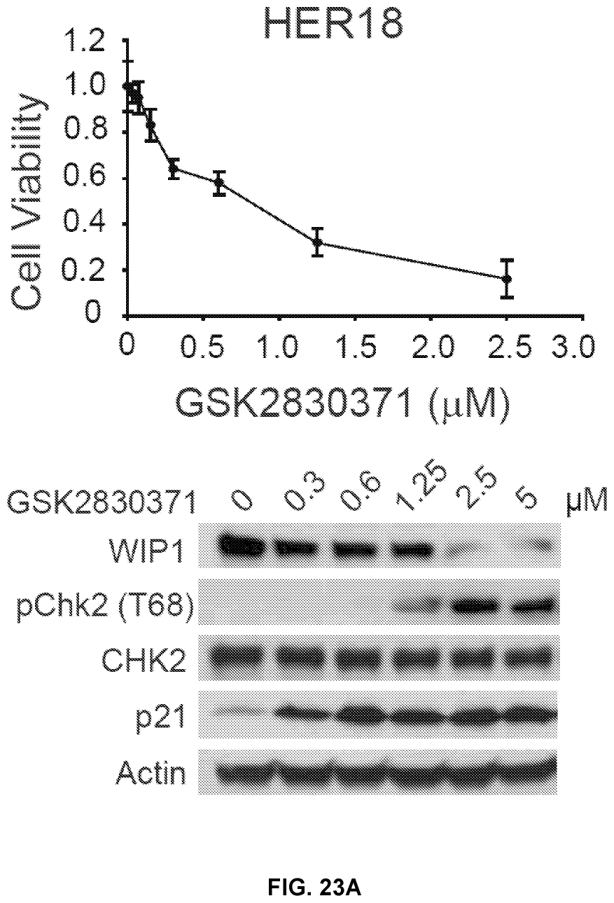

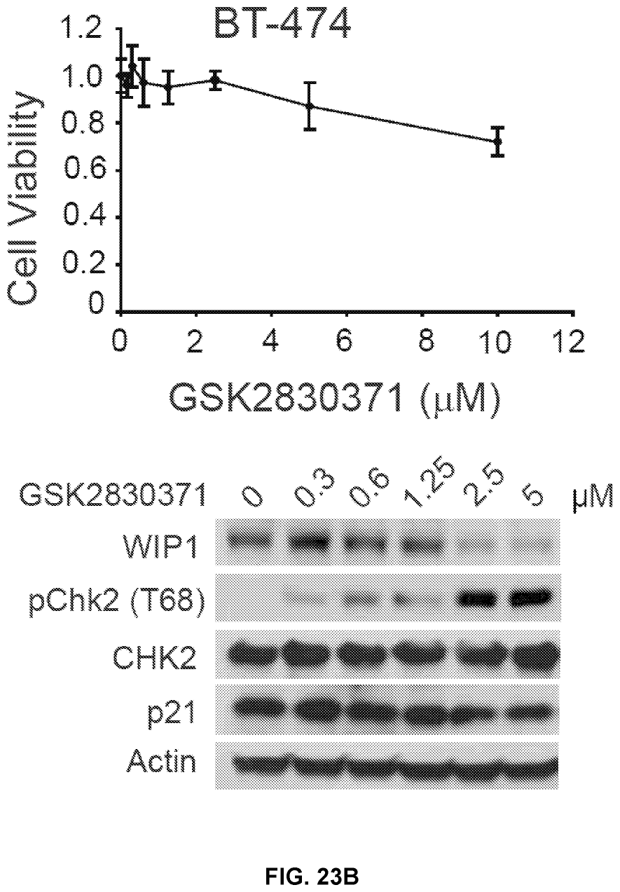

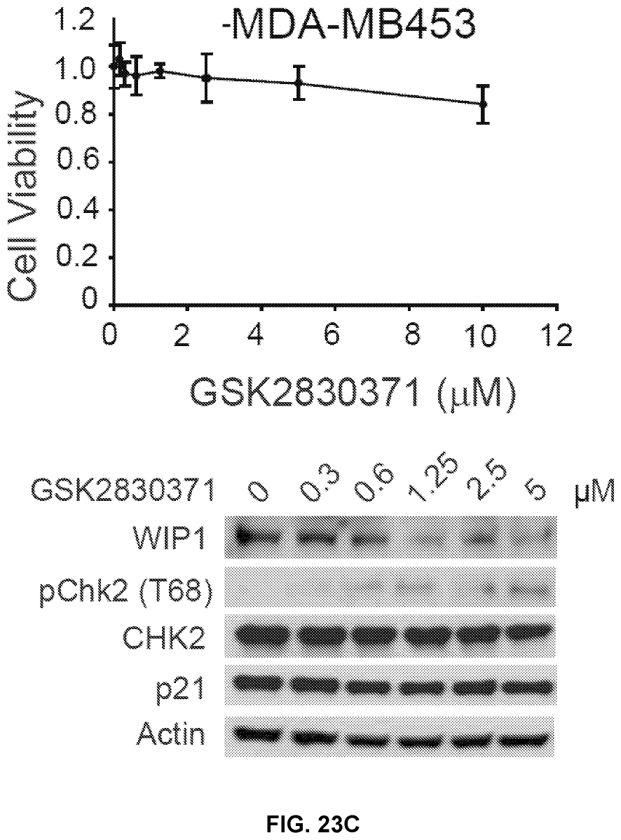

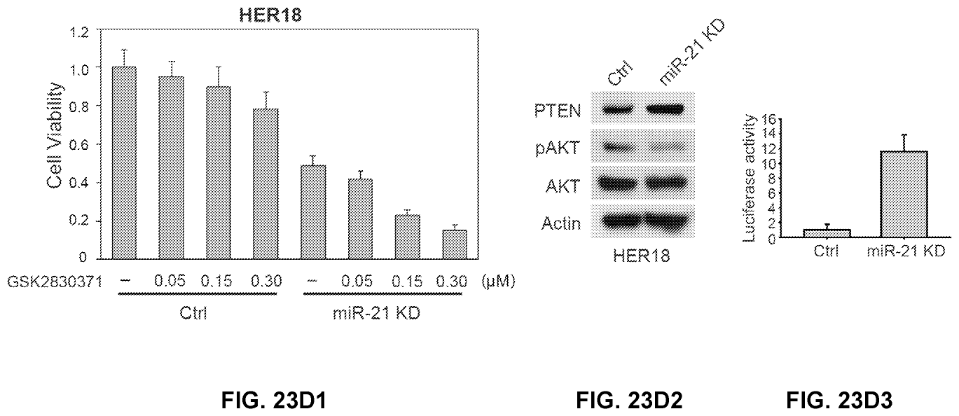

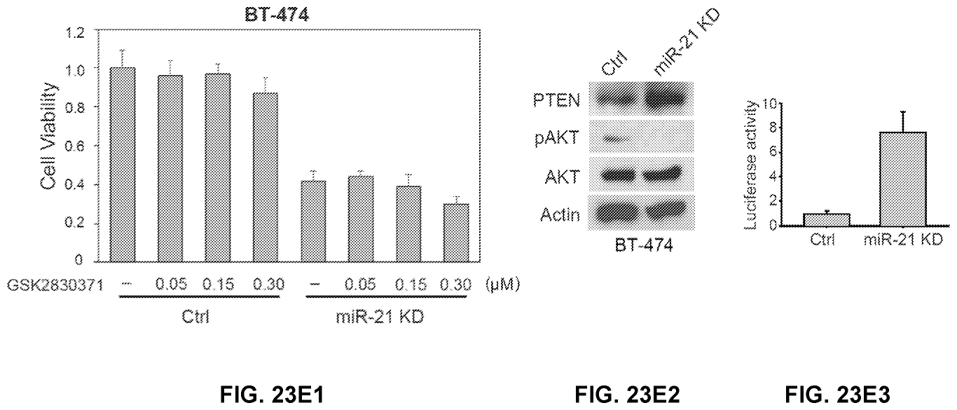

A number of studies have pointed out that p53 is an important node for the WIP1-mediated signaling network in the cell. WIP1 directly deactivates p53 by dephosphorylation of Ser15 on human p53, and promotes p53 degradation by stabilizing Mdm2. Moreover, WIP1 indirectly suppresses p53 activity by deactivating the upstream kinases including ATM, CHK1 and CHK2 in the DNA damage response. Thus, inhibiting WIP1 impacts the survival of HER2+ breast cancer cells in a p53-dependent manner. HER18 cells expressing wildtype p53 are very sensitive to the treatment of the WIP1 inhibitor GSK2830371 (FIG. 23A). Inhibition of WIP1 significantly increased the levels of phosphorylated CHK2 and the p53-induced p21 in HER18 cells. However, both of the HER2+ breast cancer cell lines with mutant p53 (BT474 and MDA-MB453) are notably insensitive to the inhibition of WIP1 (FIG. 23B, 23C), although they also have 17q23 amplicon and WIP1 overexpression led to reduced activity of DNA damage signaling, indicated by diminished levels of phosphorylated Chk2. By contrast, treatment of anti-miR-21 significantly reduced the cell proliferation and viability of the HER2+/p53-mutant cells with 17q23 amplification (FIG. 23D, 23E). Furthermore, inhibition of miR-21 dramatically sensitized HER18 cells to the treatment of the WIP1 inhibitor (FIG. 23D). These results suggest that inhibiting miR-21 is a potentially therapeutic approach to target breast cancers harboring 17q23 amplification regardless of p53 mutations.

Inhibition of miR-21 Sensitizes HER2+ Breast Cancer Cells to the Treatment of Trastuzumab

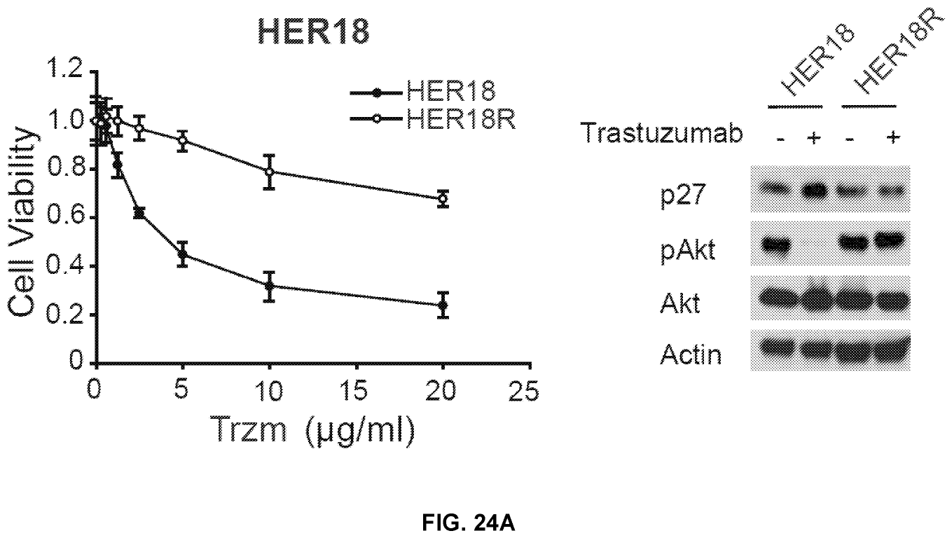

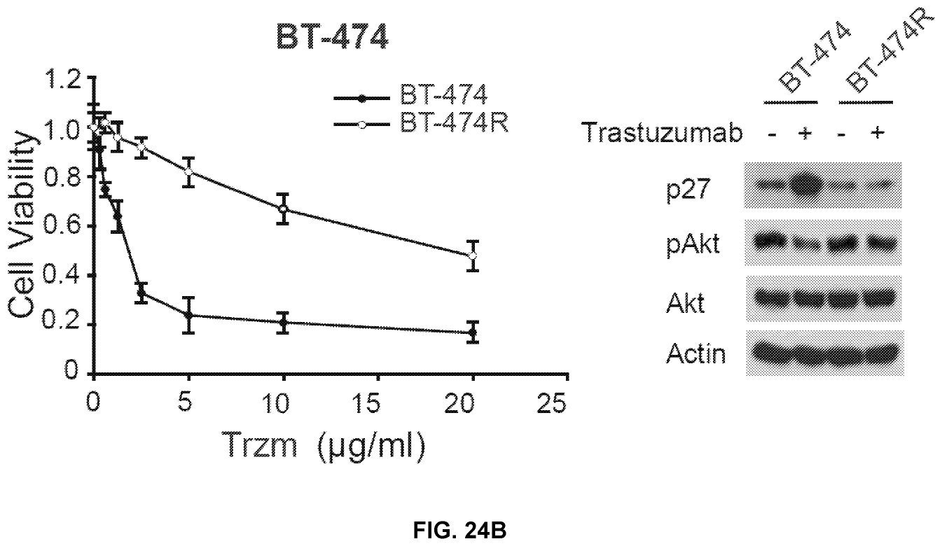

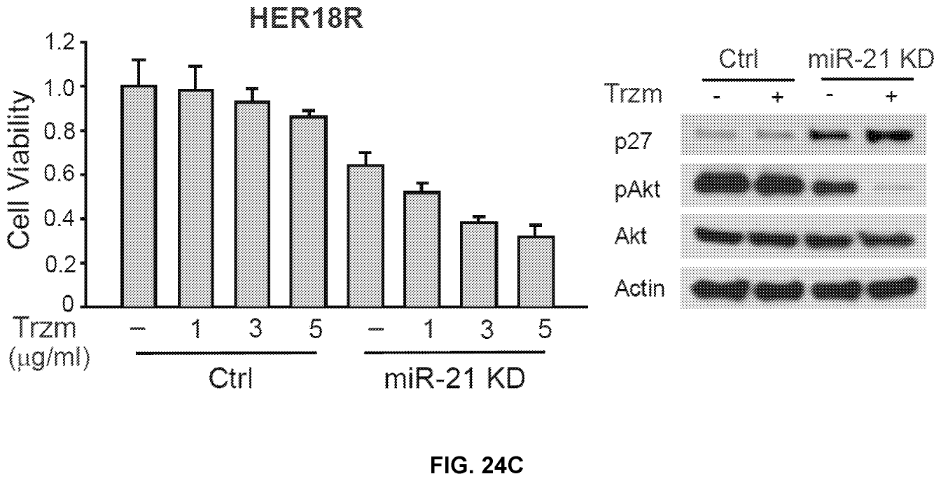

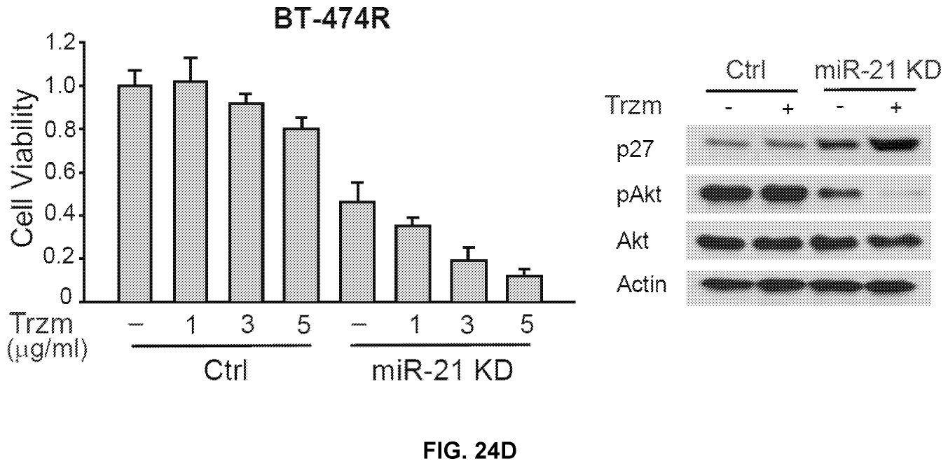

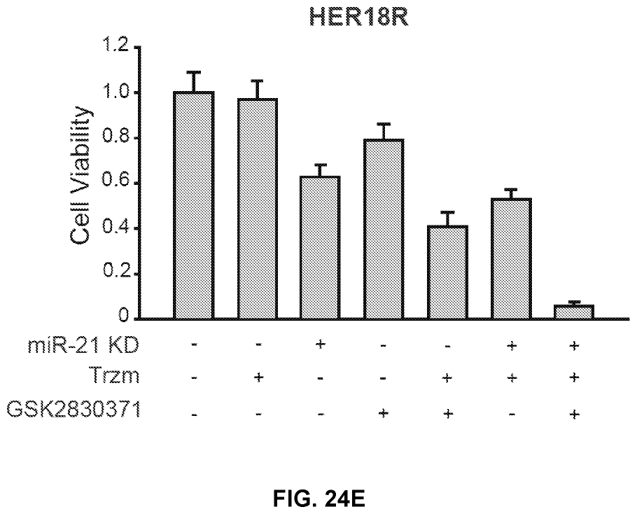

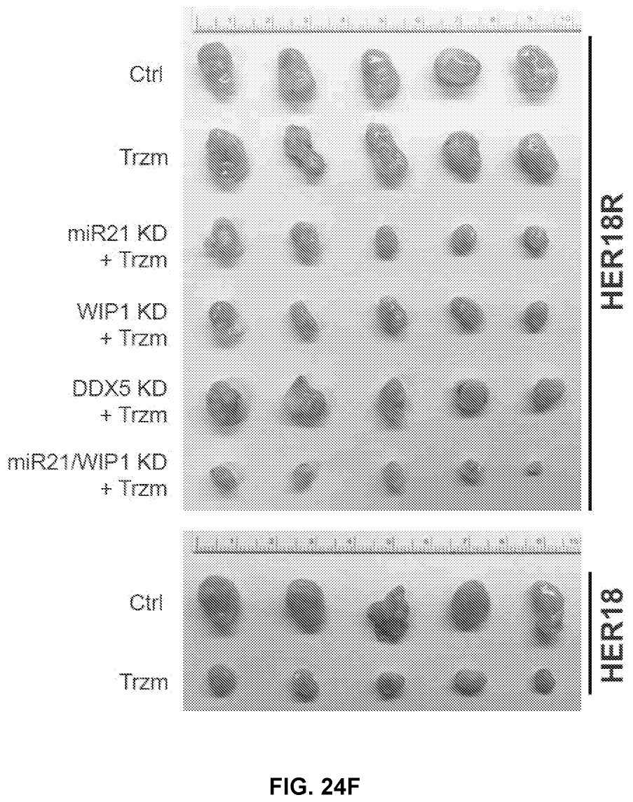

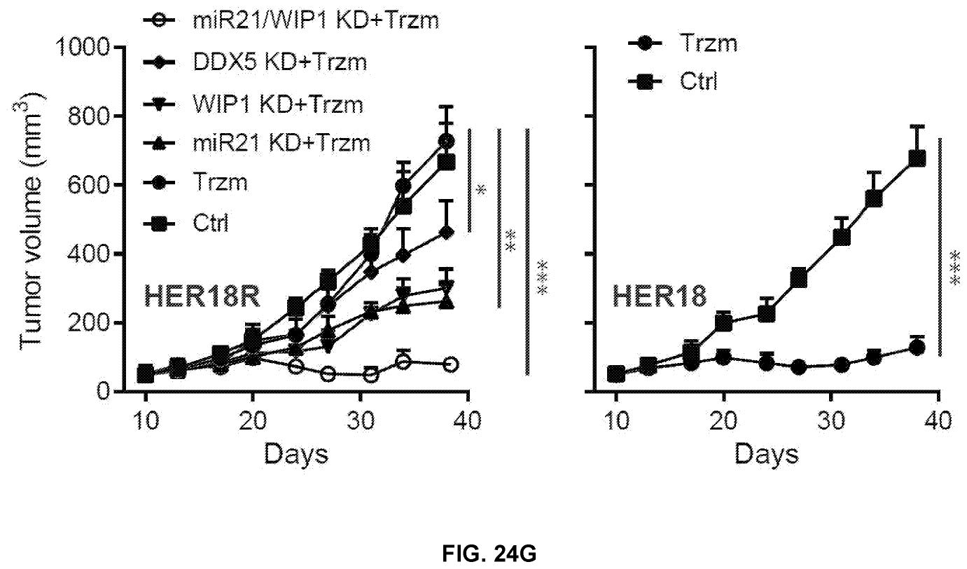

The anti-HER2 antibody trastuzumab has shown considerable clinical efficacy and extended the overall survival of patients with HER2+ breast cancer. However, multiple trastuzumab resistance mechanisms have been identified in preclinical studies, in which constitutive activation of the PI3K pathway owing to PTEN deficiency or PIK3CA mutations seems to be the most prevalent. In support of the previous reports concerning the function of miR-21 in negative regulation of PTEN (Meng F, et al. Gastroenterology 2007 133:647-658), miR-21 directly targets PTEN in mammary epithelial cells (FIG. 21A). Hence it was reasoned that inhibiting miR-21 may sensitize HER2+ breast cancer cells to the treatment of trastuzumab. Trastuzumab-resistant cell lines were generated from parental BT-474 and HER18 lines via the treatment of escalating doses up to 15 μg/ml of trastuzumab (FIG. 24A, 24B). The half maximal inhibitory concentration (IC₅₀) was increased by ˜10-fold in both resistant cell lines. Treatment of trastuzumab inhibited cell proliferation (increased p27 levels) and AKT signaling pathway (reduced pAKT levels) in parental BT-474 and HER18 cells, which were not observed in the resistant cells (BT-474R and HER18R) (FIG. 24A, 24B). Knockdown of miR-21 notably inhibited the AKT signaling as expected and suppressed cell proliferation. Moreover, miR-21 depletion markedly sensitized both BT-474R and HER18R cells to the treatment of trastuzumab, indicating the p53-independent activity of miR-21 inhibition. By contrast, knockdown of WIP1 had a similar effect on the resistant cells expressing wildtype p53 (HER18R), but not on the p53-mutant BT-474R cells (FIG. 24C, 24D). To test whether inhibiting WIP1 and miR-21 had synergistic effects on killing the trastuzumab-resistant cells, HER18R cells were treated with the WIP1 inhibitor or antagomiR-21 alone, or treated with both inhibitors, along with a low concentration of trastuzumab (1 μg/ml) (FIG. 24E). While they were insensitive to trastuzumab as single agent, HER18R cells were sensitized by miR21 inhibitor or WIP1 inhibitor alone, but the effects were drastically magnified by treatment with both inhibitors.

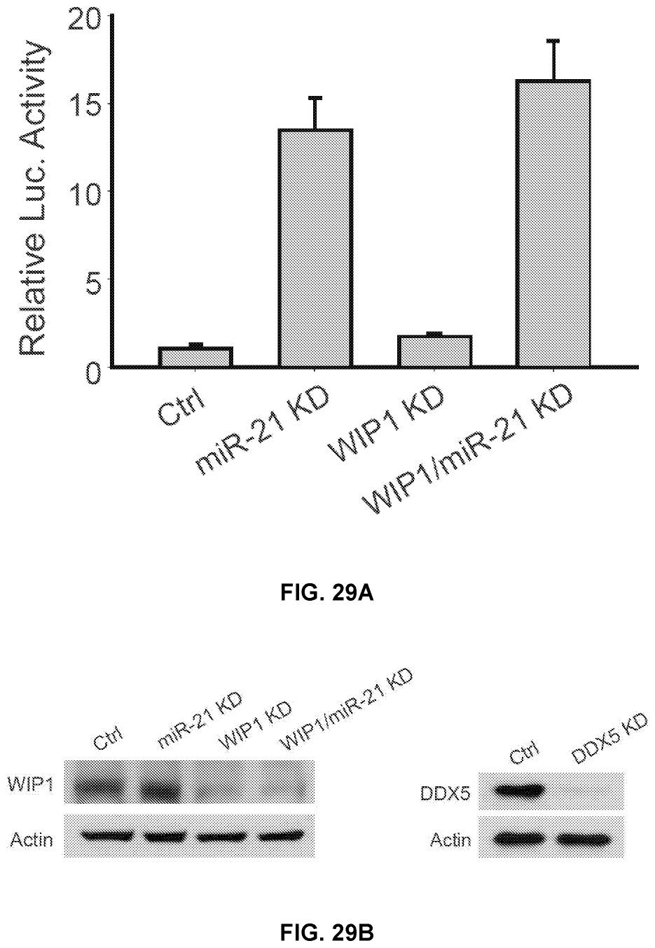

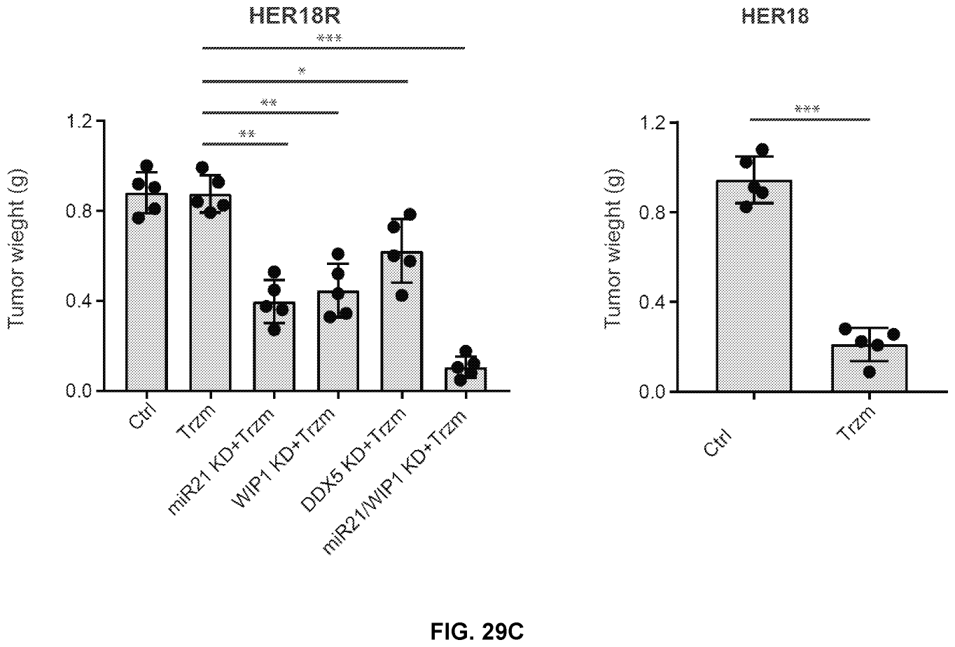

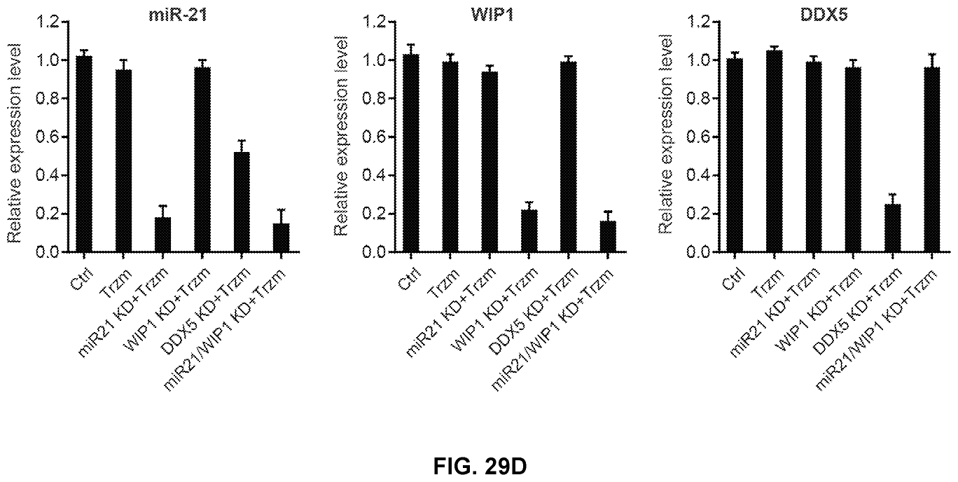

To further test the suppressive effect of WIP1 and miR-21 inhibition on breast tumor growth in vivo, HER18R cells expressing doxycycline-inducible control, WIP1, miR-21 or DDX5 short-hairpin RNA (shRNA) (>70% knockdown efficiency) were injected to mouse mammary fat pads to establish xenograft breast tumor models in female nonobese diabetic/severe combined immunodeficiency (NOD/SCID) mice. Knockdown efficiency of WIP1, miR-21, and DDX5 shRNAs was confirmed in the HER18R cells (FIGS. 29A, 29B). Once the tumors were established at three weeks after transplantation, the tumor-bearing mice were treated with 15 μg/ml of trastuzumab. The HER18R tumors were resistant to the trastuzumab treatment as expected, in comparison with the trastuzumab sensitivity of the HER18 tumors. Depletion of miR-21 or WIP1 markedly decreased the growth of xenograft tumors derived (FIGS. 24F, 29C, 29D), and their dual depletion led to more severe tumor growth inhibition, indicating synergistic roles of miR-21 and WIP1 in the trastuzumab resistance of HER2+ breast cancer. Notably, inhibition of DDX5 also exhibited modest effect on the inhibition of HER18R tumor growth, supporting its role in promoting miR-21 expression and other documented oncogenic functions (Mazurek A, et al. Cancer Discov 2012 2:812-825).

Synthesis and Characterization of Nanoparticle for Encapsulation and Delivery of miR-21 and WIP1 Inhibitors

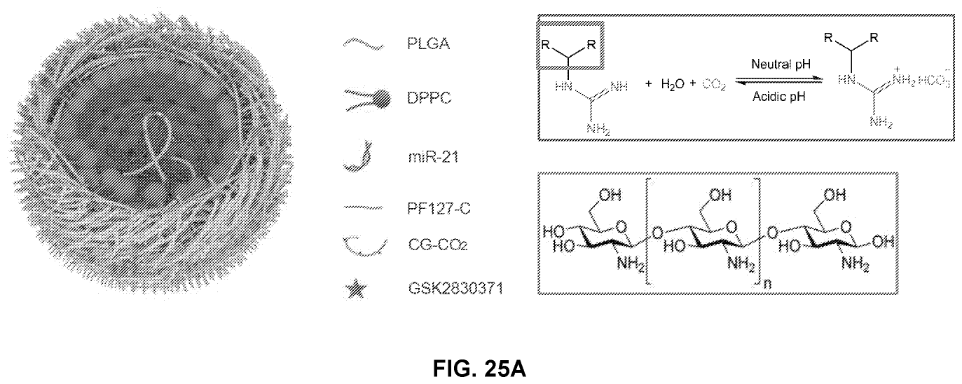

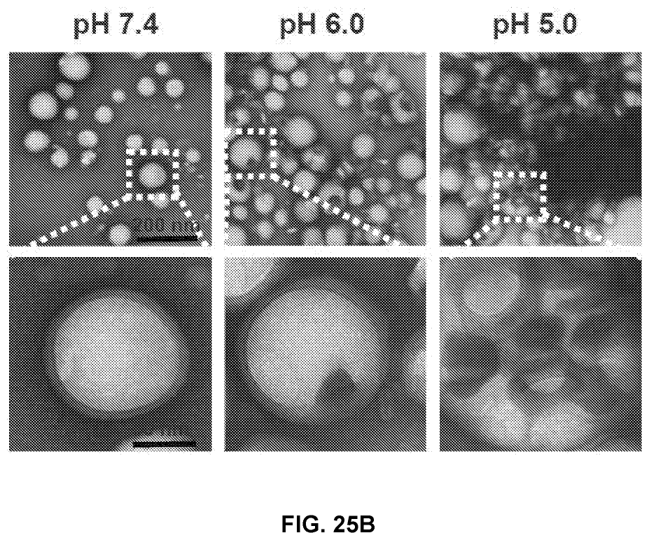

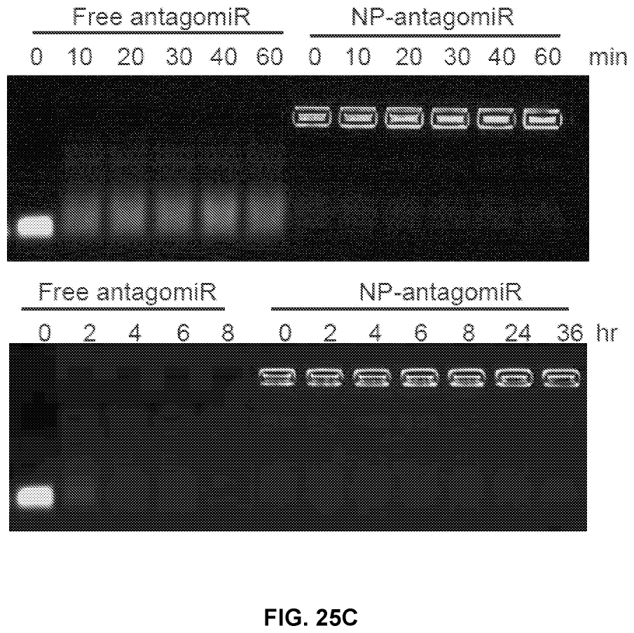

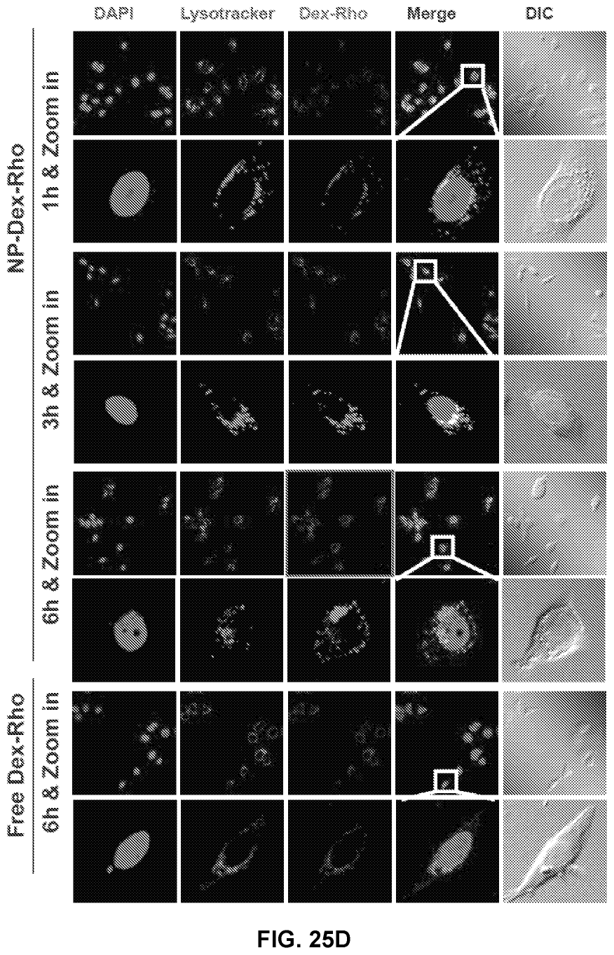

The nanoparticle was synthesized using four biocompatible materials (three polymers and one phospholipid) approved by the U.S. Food and Drug Administration (FDA) for medical use (FIG. 25A): Poly(d,l-lactide-co-glycolide) (PLGA), Pluronic F127 (PF127), chitosan, and 1,2-dipalmitoyl-sn-glycerol-3-phosphocholine (DPPC). To generate the nanobomb effect, chitosan was modified with guanidine group using a one-step reaction according to the literatures (Böttcher T, et al. J Am Chem Soc. 2013 135:2927-2930), to form chitosan-guanidine (CG). The guanidine group is common functional group in many natural products including the naturally occurring amino acid L-arginine. Importantly, the guanidine group can react reversibly with carbon dioxide (CO₂) to form chitosan-guanidinium (CG-CO₂) in a pH-dependent manner (FIG. 25A) (Seipp C A, et al. Angewandte Chemie International Edition 2017 56:1042-1045), which may be utilized to capture/store CO₂ at neutral pH for release at acidic pH. To synthesize the nanoparticle, the aqueous solution of CG was bubbled with CO₂ gas to form CG-CO₂ and then mixed with anti-miR-21 oligonucleotides. The aqueous mixture was emulsified with organic solvent or oil (dichloromethane:tetrahydrofuran 40:1) containing DPPC, PLGA, and GSK2830371 to form a water-in-oil structure, where the CG-CO₂ and oligonucleotides were encapsulated in the aqueous core while PLGA, DPPC, and GSK2830371 were in the surrounding oil. Next, the first emulsion was emulsified with the aqueous solution of PF127-Chitosan (P127 modified with chitosan (Wang H, et al. Biomaterials 2015 72:74-89), which served as the stabilizer during the second emulsion). The organic solvent was then evaporated to produce the two agents (inhibitors of miR-21 and WIP1)-laden nanoparticles (in-MW@NP).

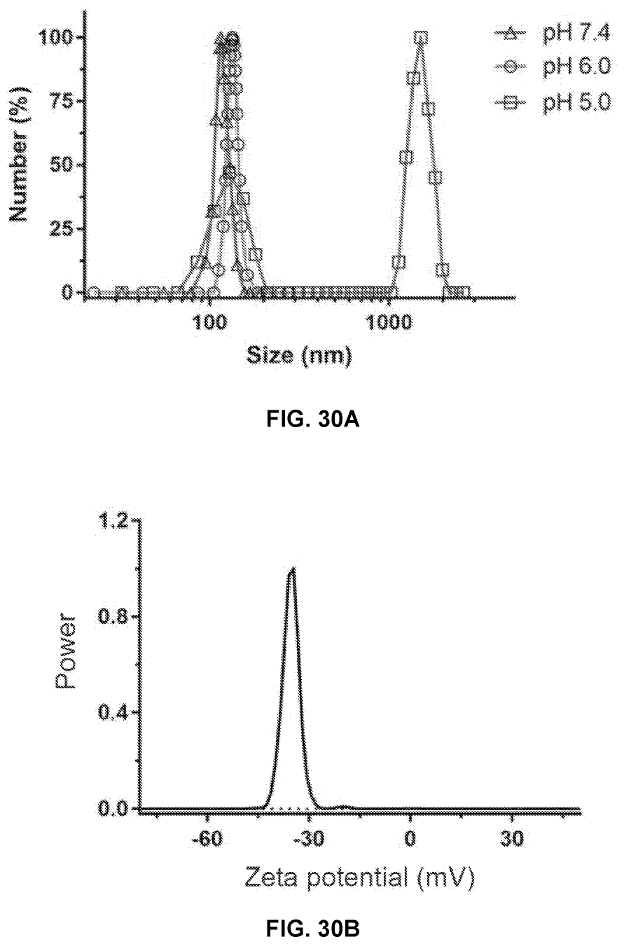

Typical transmission electron microscopy (TEM) images of in-MW@NP after soaking in Phosphate Buffer (pH 7.4, pH 6.0) or Acetate Buffer (pH 5.0), respectively, for 6 h are shown in FIG. 25B. At pH 7.4, the nanoparticles have a smooth spherical morphology and core-shell structure with diameter of 115±9.7 nm. At pH 6, defect (dark spots) can be seen on the shell of some nanoparticles. With further decrease the pH to 5, extensive defects can be seen in most of the nanoparticles and they forms large aggregates (FIGS. 25B, 30). This might be due to the low pH-activated nanobomb effect of the nanoparticles. More specifically, the CG-CO₂ encapsulated in in-MW@NP can generate/release CO₂ gas at the reduced pH to break open the nanoparticles, and the severely damaged nanoparticles may tangle together to form large aggregates.

The stability of anti-miR-21 oligonucleotide encapsulated in the nanoparticles was further examined by incubating it in medium at 37° C. for up to 36 h. As shown in FIG. 25C, the oligonucleotide encapsulated in in-MW@NP was observable even after 36 h of incubation, while the free (non-encapsulated) oligonucleotide started to degrade in a few minutes and disappeared in less than 2 h. These data show that the nanoparticle could protect the oligonucleotides from degradation by RNases in serum at neutral pH.

To study the low pH-activated nanobomb effect of in-MW@NP, Dextran (10 kD, similar to the molecular weight of anti-miR21 oligonucleotides) labeled with Rhodamine (Dex-Rho) was used to replace the anti-miR-21 oligonucleotides for synthesizing the nanoparticle (DexRho@NP). After incubating the nanoparticles with trastuzumb-resistant HER18R breast cancer cells for 1-6 h, the intracellular distribution of the red fluorescence of Rhodamine is examined against the green fluorescence of lysotracker that stains the endo/lysosomes using confocal microscopy. As shown in FIG. 25D, the red and green fluorescence largely overlaps after incubating for 1 and 3 h, indicating the cells take up the nanoparticles mainly by endocytosis. Importantly, the overlap between the red and green fluorescence is minimal at 6 h. This indicates successful escape of Dex-Rho from endo/lysosomes, probably due to the low pH-activated nanobomb effect to break open the endo/lysosomes. For comparison, cellular uptake of free Dex-Rho is minimal after 6 h of incubation and it is largely overlaps with endo/lysosomes (FIG. 25D). These data indicate that the nanoparticle encapsulation not only enhances cell uptake of Dex-Rho but also induces its endo/lysosomes escape.

Nanoparticle-Encapsulated miR-21 and WIP1 Inhibitors Effectively Suppressed the Growth of Trastuzumb-Resistant Breast Tumors

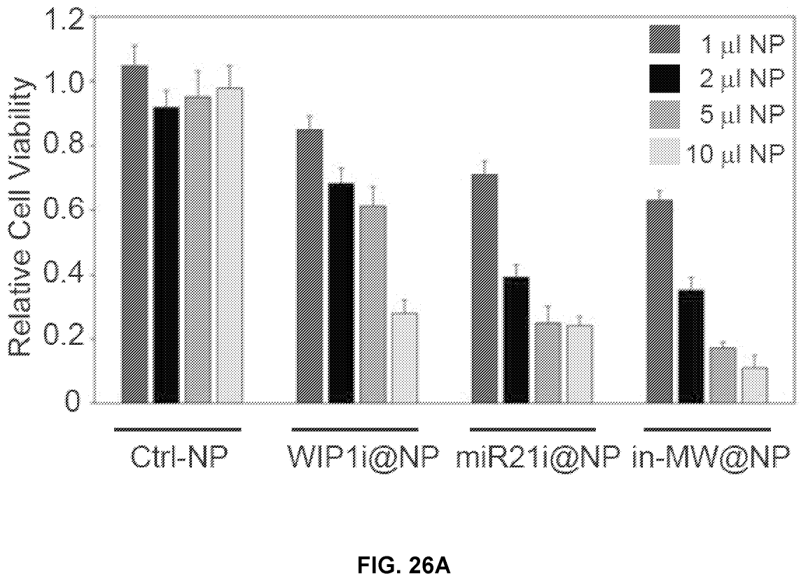

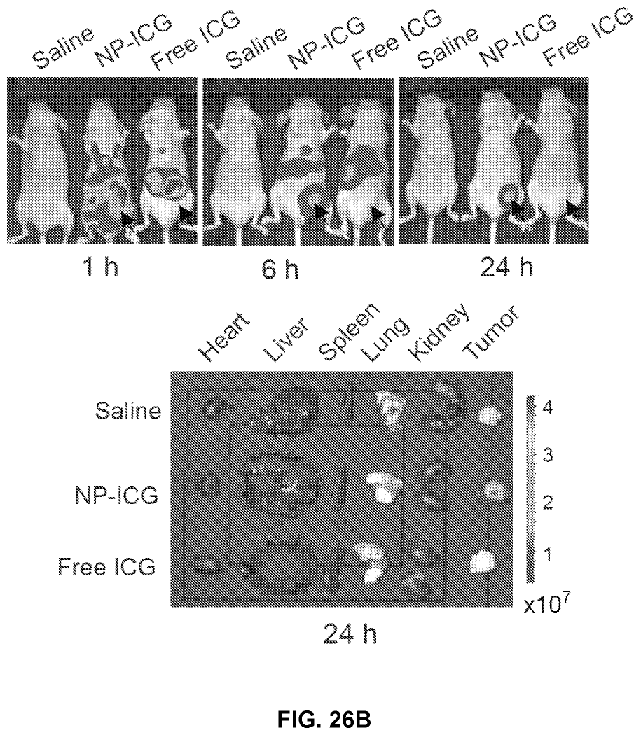

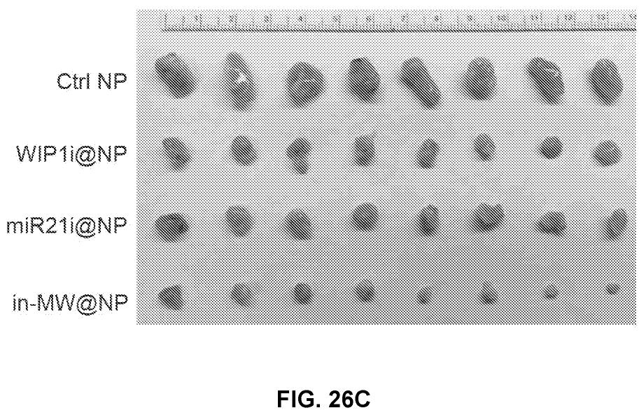

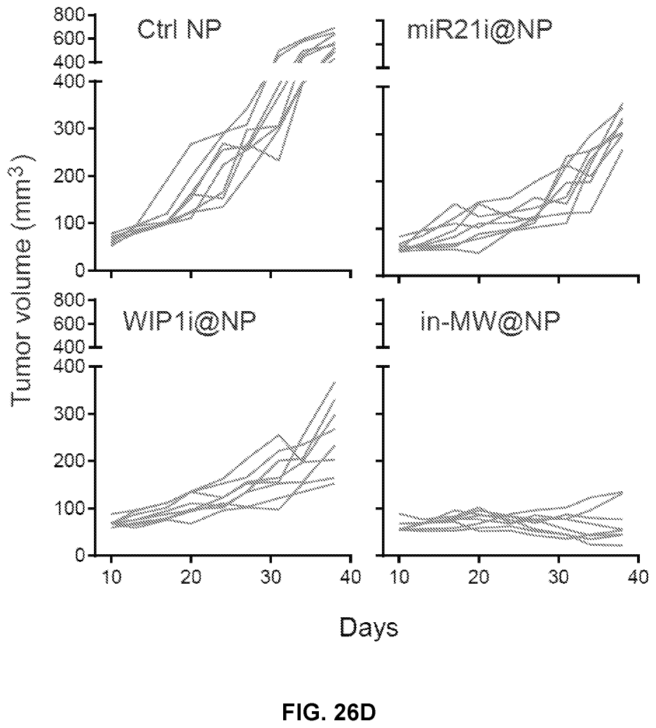

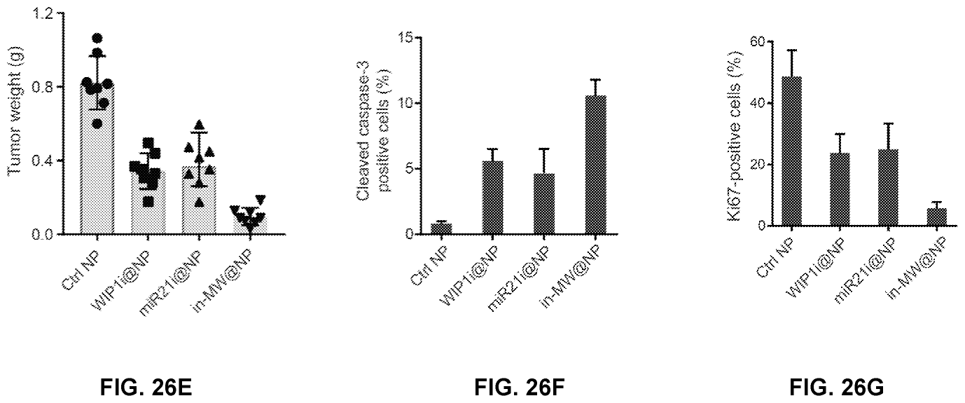

Next examined was whether the miR-21 and WIP1 inhibitors-laden nanoparticles (in-MW@NP) can be used as a nanoplatform to achieve combined therapy for effective destruction of the trastuzumb-resistant and HER2+ breast cancer cells both in vitro and in vivo. First, the anticancer capacity of the drug-laden nanoparticles was investigated in vitro (FIG. 26A). HER18R cells were treated with free WIP1/miR-21 inhibitors, blank nanoparticles, and various amounts of in-MW@NP for a total of 72 h. The cell viability was calculated by normalizing the cell number in the samples with the various treatments to the average cell number in control samples without any treatment (i.e, cultured in pure medium all the time). The blank nanoparticles without any drug were not detrimental to the cells and did not impact the cell growth for conditions with similar viability as that of control, suggesting the minimal cytotoxicity of the blank nanoparticles. The drug-laden nanoparticles are significantly more cytotoxic to the trastuzumab-resistant HER18R cells than free WIP1 or miR-21 inhibitors due to the enhanced drug delivery and release of the nanoparticles.

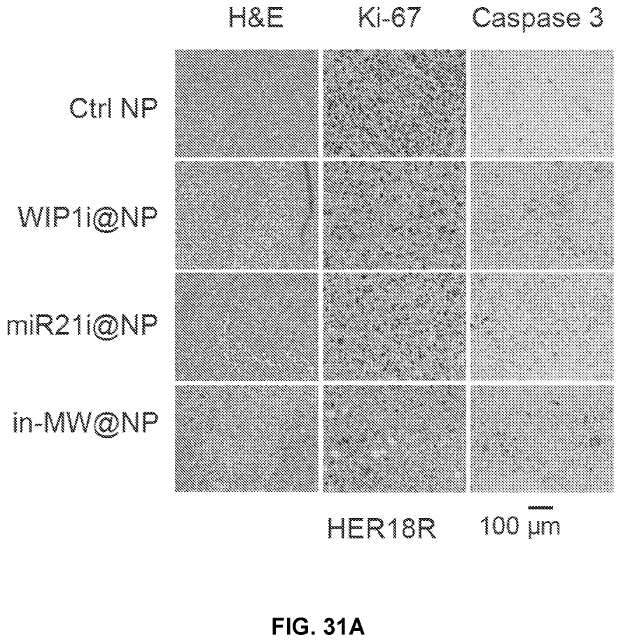

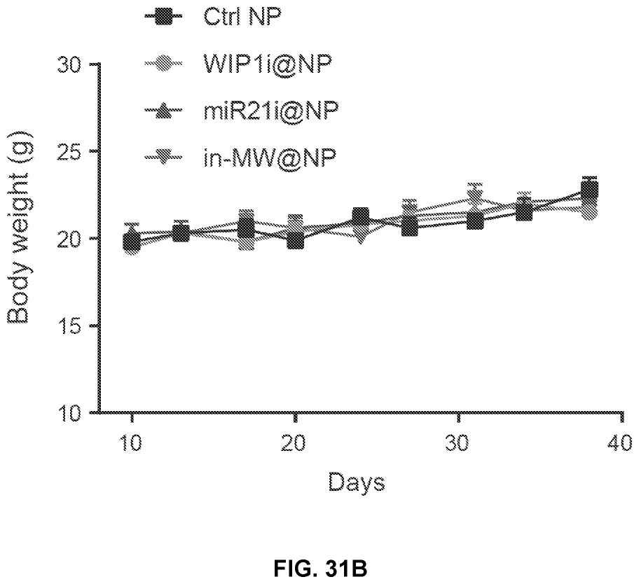

The tumor targeting capability of the nanoparticles was next investigated in mice. Indocyanine green (ICG) was encapsulated in the nanoparticles for in vivo imaging. Highly enhanced fluorescence of ICG was observable in tumor at 6 h after intravenous injection of the nanoparticles, compared to free ICG (FIG. 26B). The ICG-laden nanoparticles were exclusively localized in the tumor at 24 h, while no notable signal was seen for free ICG. To confirm the observation from whole animal imaging, various organs were harvested for ex vivo imaging to check the biodistribution of ICG after sacrificing the mice at 24 h. Indeed, only tumors from the nanoparticles treated groups showed strong fluorescence of ICG, in comparison with the tumors from the saline or free ICG-treated mice. As expected, weak fluorescence was observed in the liver from mice treated with free ICG or ICG-laden nanoparticles probably due to minimal nonspecific uptake and retention. The antitumor efficacy and safety of the WIP1 and/or miR-21 inhibitor-laden nanoparticles were also investigated in vivo by treating orthotopic HER18R mammary tumor-bearing mice. The tumors were established by injecting 2,000,000 HER18R cells per animal into the fat pad of 6-8 week-old female nude mice. The tumor-bearing mice were divided randomly into four groups: blank nanoparticles, WIP1 inhibitor-laden nanoparticles, miR-21 inhibitor-laden nanoparticles, and WIP1+miR-21 inhibitors-laden nanoparticles. Mice were treated with 1.0 mg/kg body weight of miR-21 inhibitor and/or 5.0 mg/kg of WIP1 inhibitor encapsulated in the nanoparticles via intravenous injection when the tumor reached a volume of ˜150 mm³ at 21 days after implantation. Inhibiting WIP1 or miR-21 by their inhibitor-laden nanoparticles both significantly inhibited the mammary tumor growth with ˜60-65% of reduction in tumor volumes and 45-55% of reduction in tumor weights (FIG. 26C, 26D). The combined treatment with WIP1 and miR-21 inhibitors exhibited the best anti-tumor capacity. Over 95% of tumor regression was observed in all of eight mice compared with the tumor from the control group, suggesting that inhibiting WIP1 and miR-21 is a promising therapeutic approach for trastuzumab-resistant HER2+ breast cancer. Immunohistochemical analyses showed that inhibiting miR-21 and WIP1 resulted in profound apoptosis in the HER18R tumor cells as well as significant suppression of cancer cell proliferation (FIG. 26E-26G, 31A). Moreover, side effect of the drug-laden or blank nanoparticles was negligible. Neither death nor significant drop of body weight was noted for the mice treated with saline, blank nanoparticles, and all the three drug formulations (WIP1 inhibitor, miR-21 inhibitor, and WIP1/miR-21 inhibitors) (FIG. 31B), suggesting the excellent safety of the nanoparticles for targeted delivery of WIP1 and miR-21 inhibitors in vivo.

Discussion

WIP1 is a master inhibitor of the DNA damage response. Recent studies have demonstrated that WIP1 regulates the activity and stability of a number of key players in the ATM-p53 signaling pathway (Bulavin D V, et al. Nat Genet 2002 31:210-215; Bulavin D V, et al. Nat Genet 2004 36:343-350; Li J, et al. Nat Genet 2002 31:133-134). There is substantial experimental evidence to support the oncogenic properties of WIP1, but much less is known regarding the clinical significance of WIP1 aberrations in human cancers. Although the WIP1 amplification is closely correlated with poor clinical outcome in human breast cancer, WIP1 transgene itself fails to promote tumorigenesis in mice (Gilmartin A G, et al. Nat Chem Biol 2014 10:181-187; Wong E S, et al. Dev Cell 2009 17:142-149). Here, in-depth analysis of the human 17q23 amplicon revealed that miR-21 is the other potential oncogene that may cooperate with WIP1 in mammary tumor initiation and progression. This finding is important because WIP1 and miR-21 deactivate two major tumor suppression pathways: p53 and PTEN pathways, respectively. There was also a clinically significant finding that a majority of the WIP1/miR-21-amplified cancer samples had HER2 amplification, suggesting that WIP1/miR-21 aberrations cooperate with HER2 in tumors with poor prognosis. Trastuzumab-based anti-HER2 therapy is a mainstay treatment for HER2+ breast cancer patients. While it shows considerable clinical efficacy, the overall response rate to Trastuzumab-containing therapies remains modest due to development of resistance. This study identified potential new drug targets to sensitize HER2+ breast cancer cells to trastuzumab treatment.

HER2 is amplified in 21.8% of human breast tumors. In addition, a majority of tumors with amplification of WIP1/miR-21 had HER2 amplification, suggesting that WIP1 and miR-21 may functionally interact with HER2 in human breast tumors. In clinical practice, the HER2 antibody trastuzumab and the tyrosine kinase inhibitor lapatinib are currently two primary FDA-approved drugs for the treatment of HER2-positive breast cancer. Although clinically effective, many patients with HER2+ breast cancer either do not respond or eventually develop resistance, suggesting the presence of de novo and acquired mechanisms of drug resistance. Aberrant expression of WIP1 and miR-21 may promote breast tumorigenesis by inhibiting OIS in mammary epithelial cells. OIS is a key anti-cancer barrier at the early stage of tumorigenesis, which involves the ATM-p53 and TGF-β signaling pathways in mammary glands. While the importance of the ATM-p53 signaling has been extensively studied, recent evidence shows that the TGF-β signaling is responsible for the ATM-independent OIS in mammary glands (Cipriano R, et al. Proc Natl Acad Sci USA 2011 108:8668-8673). Although WIP1 is a master inhibitor in the ATM-p53 signaling, overexpression of WIP1 in transgenic mice does not induce mammary tumorigenesis, suggesting that intact TGF-β signaling needs to be overcome for breast cancer initiation. miR-21 targets a number of key genes that induces TGF-β signaling and thus miR-21 is a probably potent inhibitor in the TGF-β pathway. These results suggest that WIP1 and miR-21 cooperatively inhibit these two pathways to override OIS and promote mammary tumorigenesis.

Post-transcriptional processing of pri-miRNAs is an essential step in miRNA biogenesis. While Drosha and DGCR8 are the core components in the microprocessor, neither of them has binding specificity for individual pri-miRNAs (Bartel D P. Cell 2009 136:215-233; Lin S, et al. Nat Rev Cancer 2015 15:321-333; Chendrimada T P, et al. Nature 2005 436:740-744). DDX5 was identified as a miR-21-specific regulator in the Drosha microprocessor. Thus, co-amplification of DDX5 with MIR21 facilitates the efficient processing of primary miR-21 transcripts and results in elevated levels of miR-21. This hypothesis is also supported by the positive correlation between DDX5 copy numbers and miR-21 levels in mammary tumor tissues. In addition to its role in miRNA processing, DDX5 is an essential gene in the development as DDX5 knockout mice are embryonically lethal. By its interaction with mRNA, DDX5 is also involved in the processing, splicing and degradation of mRNA. DDX5 directly regulates DNA replication factor expression by promoting the recruitment of RNA polymerase II to E2F-regulated gene promoters. DDX5 was suggested as a promising candidate for targeted therapy of breast tumors with DDX5 amplification. Inhibiting DDX5 suppressed the growth of HER2+ tumors in vivo. In the HER18-derived breast tumor models, direct inhibition of miR-21 or WIP1 had greater effects on suppressing tumor growth, suggesting that DDX5 is likely one of the important regulators for mammary tumors induced by 17q23 amplification.

Alterations in the PTEN/PI3K/AKT pathways are cited as contributors to the development of trastuzumab resistance, however targeting these kinases as single agents has yielded less than expected clinical results. Other pathways, such as Ras/MAPK pathway, which are typically not mutationally activated in breast cancer, were shown to contribute to the trastuzumab resistance. Whereas miR-21 inhibits PTEN signaling, WIP1 is a broad inhibitor of the MAPK and ATM-p53 pathways. Herein, inhibiting miR-21 and WIP1 can be developed into a specified therapy for HER2+ breast cancer harboring 17q23 amplicon.

Although RNA interference (RNAi) has attracted a lot of attention as a promising therapeutic strategy for cancer in the past decades, few RNAi-based therapies have passed/entered Phase II/III clinical trial (Wittrup A, et al. Nature Reviews Genetics 2015 16:543; Bobbin M L, et al. Annu Rev Pharmacol Toxicol. 2016 56:103-122; Dahlman J E, et al. Nature Nanotechnology 2014 9:648). This is partly because naked RNAs such as anti-miR have poor stability in blood, do not enter cells, and are instable in the endo/lysosome inside cells (Wang H, et al. Adv Mater 2016 28:347-355). In addition, the small molecule drugs for conventional chemotherapy is either insoluble in water or can diffuse to both normal tissue and tumor, which may induce significant side effects (Wang H, et al. Nanomedicine 2016 11:103-106). Nanotechnology have demonstrated great potential for overcoming the challenges facing conventional chemo/RNAi therapy (Wang H, et al. Nanomedicine 2016 11:103-106; Cui J, et al. Nature communications 2017 8:191; Zuckerman J E, et al. Nat Rev Drug Discov. 2015 14:843). In this study, a unique nanoparticle was designed and synthesized to encapsulate both WIP1 and miR-21 inhibitors for combination therapy. It not only can improve the solubility and bioavailability of GSK2830371 for inhibiting WIP1 but also achieve cytosolic delivery of anti-miR21, to deactivate tumor suppression p53 and PTEN pathways. The nanoparticles could stabilize anti-MiR21 by preventing it from the enzymatic degradation during circulation and preferentially accumulate/target tumor. After entering tumor, it could enhance cellular uptake of the encapsulated agents. More importantly, after being taken up by cancer cells via endocytosis, the nanoparticle could generate carbon dioxide gas to break open endo/lysosomes. Moreover, the disclosed nanoparticle-based approach ensures that the dose ratio of the two agents in the tumor can be maintained to be the same as that at injection, while the dose ratio of the two agents in tumor may be very different from that at injection due to vast difference in bioavailability of the hydrophobic GSK2830371 and hydrophilic anti-miR21 oligonucleotide. This well-designed nanoparticle is an excellent vehicle for delivering the anti-miR21 (and other RNAs) to overcome both the extracellular and intracellular barrier to the use of RNAs for cancer therapy. By taking advantage of advanced nanotechnology, a strategy is presented that involves using pH-responsive nanoparticle to inhibit WIP1 and miR-21 for effective therapy of trastuzumab-resistant HER2+ breast cancer harboring 17q23 amplicon.

In summary, co-amplification of MIR21 and WIP1 in HER2+ breast cancer harboring 17q23 amplicon generates therapeutic vulnerabilities and provides an effective treatment strategy for breast cancers containing such genomic events. miR-21 and WIP1 functionally cooperate with the HER2 gene in breast tumorigenesis, and inhibiting them circumvents resistance to anti-HER2 therapies.

### Example 3: Synthesis of Active CSC and Tumor Dual-Targeting, Low pH-Activated Nanobomb Co-Encapsulated with siPol2 (R) and PTX (P)

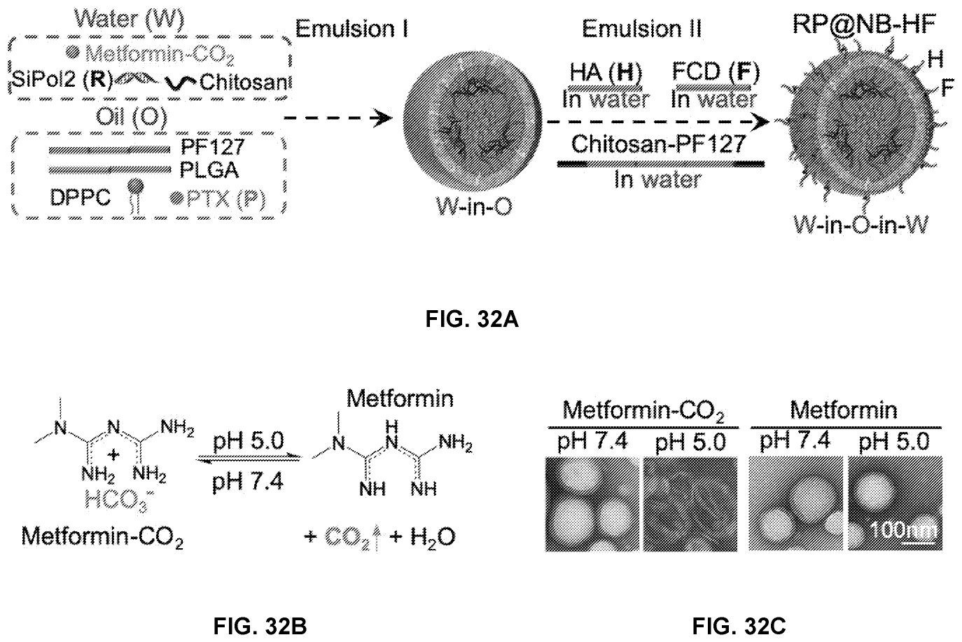

As illustrated in FIG. 32A, the dual-targeting low pH-activated nanobomb (RP@NB-HF) are made using an alternative double emulsion approach (ADEA) and polymers (PF127, PLGA, DPPC, chitosan-PF127, chitosan, and HA) with the following slight modifications:

First, ICG and ammonium bicarbonate (required for the NIR laser-activated bomb effect) are replaced with metformin carbonate/bicarbonate (metformin-CO₂, FIG. 32B). Chitosan is still used to improve the encapsulation efficiency of siPol2. Metformin can bind with CO₂ at neutral pH and release it at low pH, to give the low pH-activated bomb effect similar to that of CG-CO₂. Metformin-CO₂ is made by bubbling the aqueous metformin solution at pH 7.4 with CO₂ gas. By keeping the CO₂ amount the same and replacing CG-CO₂ with metformin-CO₂, the resultant nanoparticles (containing metformin-CO₂) appear damaged after incubating at pH 5.0 for 3 h (FIG. 32C). In contrast, if metformin (without CO₂) is used, the resultant nanoparticles appears intact at pH 5.0 (FIG. 32C). Metformin has the advantage of being a clinical drug for diabetes and is effective for killing CSCs.

Second, PTX (P) is dissolved in oil (i.e, dichloromethane) during emulsion I for encapsulation into the hydrophobic shell of the resultant nanoparticles. Third, fucoidan (FCD or F) is added together with HA (H) into the aqueous phase for emulsion 11 so that it can be decorated on the nanoparticle surface together with HA, as illustrated in FIG. 32A. The concentration of FCD and HA is adjusted for optimal tumor and CSC targeting. The amount of the agents encapsulated in the nanoparticles is quantified using HPLC or colorimetry. Release of the agents from the nanoparticles is studied at pH 7.4, 6.0, and 5.0. The feeding amount of each agent is adjusted to achieve the desired dose ratio of the agents in the nanobomb.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

1. A pH activated nanoparticle, comprising
a shell comprising a phospholipid bilayer, and
a core comprising a gas bound to a substrate by a pH sensitive interaction.

2. The nanoparticle of claim 1, wherein the substrate comprises chitosan-guanidine (CG) or chitosan-arginine (CA).

3. The nanoparticle of claim 1, wherein the substrate comprises metformin.

4. The nanoparticle of claim 1, wherein the substrate comprises calcium carbonate.

5. The nanoparticle of claim 1, wherein the gas comprises carbon dioxide.

6. The nanoparticle of claim 1, wherein the core further comprises a pH sensitive therapeutic or diagnostic agent.

7. The nanoparticle of claim 6, wherein the pH sensitive therapeutic or diagnostic agent is an RNA or DNA oligonucleotide.

8. The nanoparticle of claim 7, wherein the pH sensitive therapeutic or diagnostic agent is an mRNA, ncRNA, siRNA, miRNA, or shRNA oligonucleotide.

9. The nanoparticle of claim 6, wherein the pH sensitive therapeutic or diagnostic agent is peptide.

10. The nanoparticle of claim 6, wherein the pH sensitive therapeutic or diagnostic agent is a labile small molecule.

11. The nanoparticle of claim 7, wherein the pH sensitive therapeutic agent is a POLR2A-targeting siRNA (siPol2).

12. The nanoparticle of claim 7, wherein the pH sensitive therapeutic agent is an anti-miR-21 oligonucleotide.

13. The nanoparticle of claim 12, further comprising a small molecule inhibitor against WIP1.

14. The nanoparticle of claim 13, wherein the small molecule inhibitor against WIP1 comprises GSK2830371.

15. The nanoparticle of claim 1, further comprising paclitaxel, camptothecin, doxorubicin, or any combination thereof.

16. The nanoparticle of claim 1, wherein the phospholipid bilayer comprises dipalmitoyl phosphatidylcholine (DPPC) or dioleoyl phosphatidylcholine (DOPC).

17. The nanoparticle of claim 1, wherein the shell further comprises poly(lactic-co-glycolic acid) (PLGA).

18. The nanoparticle of claim 17, wherein the PLGA is PEGylated.

19. The nanoparticle of claim 1, wherein the shell further comprises a poloxamer.

20. The nanoparticle of claim 19, wherein the poloxamer is poloxamer 407.

21. A method for treating triple negative breast cancer (TNBC) in a subject, comprising administering to the subject a therapeutically effective amount of the pH activated nanoparticle of claim 11.

22. The method of claim 21, wherein the TNBC has a TP53 gene mutation or deletion.

23. A method for delivering a pH sensitive cargo to the cytoplasm of a cell, comprising loading the pH sensitive cargo into the pH activated nanoparticle of claim 1, and contacting the cell with the loaded nanoparticle.

24. A method for treating HER2+ breast cancer in a subject, comprising administering to the subject a therapeutically effective amount of an anti-miR-21 oligonucleotide and a small molecule inhibitor against WIP1.

25-32. (canceled)


SEQUENCE LISTING (29 sequences)

SEQ ID NO: 1 - RNA, 19 nt, Artificial Sequence
  Synthetic Construct
ccaacaugcugacagauau

SEQ ID NO: 2 - RNA, 19 nt, Artificial Sequence
  Synthetic Construct
auaucugucagcauguugg

SEQ ID NO: 3 - RNA, 19 nt, Artificial Sequence
  Synthetic Construct
ccaagaagcggcucacaca

SEQ ID NO: 4 - RNA, 19 nt, Artificial Sequence
  Synthetic Construct
ugugugagccgcuucuugg

SEQ ID NO: 5 - RNA, 23 nt, Artificial Sequence
  Synthetic Construct
ucaacaucagucugauaagcuag

SEQ ID NO: 6 - DNA, 20 bp, Artificial Sequence
  Synthetic Construct
ttttgttttgcttgggagga

SEQ ID NO: 7 - DNA, 20 bp, Artificial Sequence
  Synthetic Construct
agcagacagtcaggcaggat

SEQ ID NO: 8 - DNA, 23 bp, Artificial Sequence
  Synthetic Construct
atgttgactgttgaatctcatgg

SEQ ID NO: 9 - DNA, 19 bp, Artificial Sequence
  Synthetic Construct
tgtcagacagcccatcgac

SEQ ID NO: 10 - DNA, 20 bp, Artificial Sequence
  Synthetic Construct
gatgcaagggtcagaagggc

SEQ ID NO: 11 - DNA, 19 bp, Artificial Sequence
  Synthetic Construct
gagccatctggcccggacg

SEQ ID NO: 12 - DNA, 26 bp, Artificial Sequence
  Synthetic Construct
gcaattacagtattttaagagatgat

SEQ ID NO: 13 - DNA, 26 bp, Artificial Sequence
  Synthetic Construct
catactctacagttgtgttttaatgt

SEQ ID NO: 14 - DNA, 22 bp, Artificial Sequence
  Synthetic Construct
ttgatgggaaagttacctacgg

SEQ ID NO: 15 - DNA, 22 bp, Artificial Sequence
  Synthetic Construct
caagatgcaggaaagatgtctg

SEQ ID NO: 16 - DNA, 24 bp, Artificial Sequence
  Synthetic Construct
cacagcatacactttctttacacc

SEQ ID NO: 17 - DNA, 20 bp, Artificial Sequence
  Synthetic Construct
tggaacgacctgaacctctg

SEQ ID NO: 18 - DNA, 19 bp, Artificial Sequence
  Synthetic Construct
agccacatcgctcagacac

SEQ ID NO: 19 - DNA, 19 bp, Artificial Sequence
  Synthetic Construct
gcccaatacgaccaaatcc

SEQ ID NO: 20 - DNA, 21 bp, Artificial Sequence
  Synthetic Construct
agaagcagaagggtttcacct

SEQ ID NO: 21 - DNA, 21 bp, Artificial Sequence
  Synthetic Construct
cattccgccagtttcttccac

SEQ ID NO: 22 - DNA, 20 bp, Artificial Sequence
  Synthetic Construct
tgcagggaaacctggaactc

SEQ ID NO: 23 - DNA, 21 bp, Artificial Sequence
  Synthetic Construct
acaggggtggtattgttcagc

SEQ ID NO: 24 - DNA, 51 bp, Artificial Sequence
  Synthetic Construct
ggtggcatcaaatacccagagacgactgagggaggccgccccaagcttggg

SEQ ID NO: 25 - protein, 17 aa, Artificial Sequence
  Synthetic Construct
GGIKYPETTEGGRPKLG

SEQ ID NO: 26 - DNA, 43 bp, Artificial Sequence
  Synthetic Construct
ggtggcatcaaatacccagagacgaggccgccccaagcttggg

SEQ ID NO: 27 - DNA, 54 bp, Artificial Sequence
  Synthetic Construct
tgattgagcggactggccgctgccaaacatgtgcaggaaacatgacagagtgtc

SEQ ID NO: 28 - protein, 18 aa, Artificial Sequence
  Synthetic Construct
VIERTGRCQTCAGNMTEC

SEQ ID NO: 29 - DNA, 50 bp, Artificial Sequence
  Synthetic Construct
tgattgagcggactggccgctgccaaacataggaaacatgacagagtgtc